(19) United States
(12) Patent Application Publication
(10) Pub. No.: US 2023/0287070 A1
(43) Pub. Date: Sep. 14, 2023

(54) NOVEL TUMOR-SPECIFIC ANTIGENS FOR ACUTE MYELOID LEUKEMIA (AML) AND USES THEREOF

(71) Applicant: UNIVERSITE DE MONTREAL, Montreal (CA)

(72) Inventors: Claude Perreault, Montreal (CA); Pierre Thibault, Montreal (CA); Sebastien Lemieux, Lasalle (CA); Gregory Ehx, Liege (BE); Marie-Pierre Hardy, Terrebonne (CA)

(73) Assignee: UNIVERSITE DE MONTREAL, Montreal (CA)

(21) Appl. No.: 17/916,539

(22) PCT Filed: Mar. 15, 2021
(86) PCT No.: PCT/CA2021/050340

§ 371 Date: Sep. 30, 2022

Related U.S. Application Data
(60) Provisional application No. 63/009,853, filed on Apr. 14, 2020.

(51) Int. Cl. C07K 14/47; C07K 14/725; A61P 35/02

(52) U.S. Cl. CPC C07K 14/4748; A61P 35/02; C07K 14/7051; A61K 39/00

(57) ABSTRACT

Acute myeloid leukemia (AML) has not benefited from innovative immunotherapies, mainly because of the lack of actionable immune targets. Novel tumor-specific antigens (TSAs) shared by a large proportion of AML cells are described herein. Most of the TSAs described herein derives from aberrantly expressed unmutated genomic sequences, such as intronic and intergenic sequences, which are not expressed in normal tissues. Nucleic acids, compositions, cells and vaccines derived from these TSAs are described. The use of the TSAs, nucleic acids, compositions, cells and vaccines for the treatment of leukemia such as AML is also described.

### CROSS REFERENCE TO RELATED APPLICATIONS

The present application claims the benefits of U.S. Provisional Pat. Application No. 63/009,853 filed on Apr. 14, 2020, which is incorporated herein by reference in its entirety.

### SEQUENCE LISTING

N/A.

### TECHNICAL FIELD

The present invention generally relates to cancer, and more specifically to tumor antigens specific for acute myeloid leukemia useful for T-cell-based cancer immunotherapy.

### BACKGROUND ART

Acute myeloid leukemia (AML), the most aggressive hematologic malignancy, is a heterogeneous disease characterized by aberrant epigenetic patterning, disturbed mitochondrial proteostasis and a relatively low number of mutations (Li et al., 2016; Ntziachristos et al., 2016; Ishizawa et al., 2019; Fennell et al., 2019). Notably, genetic and epigenetic changes in AML may precede diagnosis by many years (Abelson et al., 2018; Desai et al., 2018). Also, cure requires not only elimination of bulk tumor cells but also of leukemic stem cells (Shlush et al., 2017; Boyd et al., 2018). Currently, most patients relapse following chemotherapy, with 5-year overall survival of 40% for patients <60 years and only 10-20% for those aged ≥60 years (who represent the majority of AML cases) (Vasu et al., 2018).

Over the last few years, enthusiasm for cancer immunotherapy has been fueled mainly by two major breakthroughs: i) immune checkpoint therapy for treatment melanoma and selected types of solid tumors and ii) chimeric antigen receptors for treatment of lymphoid malignancies. However, AML has not benefited from such innovations, mainly because of the lack of actionable immune targets. In accordance with the notion that major histocompatibility complex MHC-associated peptides (MAPs) recognized by T cells are at the core of anti-cancer responses (Coulie et al., 2014), evidence suggests that AML cells should present immunogenic MAPs to CD8 T cells: i) AML cells express a high density of MHC class I molecules (Berlin et al., 2015) and ii) the bone marrow of AML patients contains CD8 T cells with phenotypic and transcriptional features of exhaustion (and therefore of antigen recognition) (Knaus et al., 2018). However, the nature of AML antigens able to elicit protective immune responses remains elusive.

The first class of MAPs that attracted the attention of cancer immunologists are tumor-associated antigens (TAAs) that are overexpressed in tumor cells relative to normal cells. Since high-affinity T cells recognizing self-antigens are eliminated by the central tolerance process of thymic selection, TAAs are essentially recognized by low affinity T cells. Accordingly, TAA-based vaccines have had no convincing impact on AML evolution. Disappointing results were notably obtained with the most studied AML TAA: Wilms’ Tumor 1 (WT1) (Di Stasi et al., 2015; Maslak et al., 2018; Rashidi and Walter, 2016). Importantly, a recent report showed that TCR gene therapy (in which T cells are engineered to express a high affinity TCR against a selected antigen) targeted against a WT1-derived peptide could durably prevent relapse in recipients of allogeneic hematopoietic stem cell transplantation (Chapuis et al., 2019). Overall, these studies suggest that WT1-derived peptides are poorly immunogenic and need to be targeted with engineered T cells to reach their full therapeutic potential.

In contrast to TAAs, tumor specific antigens (TSAs) are MAPs solely presented by tumor cells. So far, mutated TSAs (mTSAs), also known as neoantigens, have attracted substantial attention lately in quests for vaccines against solid tumors. Indeed, mTSAs can be highly immunogenic because they are not found in medullary thymic cells (mTECs) which induce central tolerance. However, mTSAs present two caveats. First, they are generally unique to each patients’ tumors (private neoantigens). Second, they are less common than initially predicted (Knaus et al., 2018). Consistent with the low mutational burden of AML cells, only one mTSA has been validated by mass spectrometry (MS) analyses of primary AML cells (van der Lee et al., 2019). The therapeutic potential of this mTSA deriving from a frameshift in the NPM1 gene has yet to be evaluated, but according to available evidence, it does not elicit spontaneous immune responses in AML patients (van der Lee et al., 2019).

In view of this, there is a pressing need to identify the antigens that can elicit therapeutic immune responses again AML. Such antigens could be used as vaccines (± immune checkpoint inhibitors) or as targets for T-cell receptor-based approaches (cell therapy, bispecific biologics).

The present description refers to a number of documents, the content of which is herein incorporated by reference in their entirety.

### SUMMARY OF THE INVENTION

The present disclosure provides the following items 1 to 67:

1. A leukemia tumor antigen peptide (TAP) comprising one of the following amino acid sequences:

| | | | |
|---|---|---|---|
| Sequence (SEQ ID NO) | Sequence (SEQ ID NO) | Sequence (SEQ ID NO) | Sequence (SEQ ID NO.) |
| RQISVQASL (1) | SIQRNLSL (49) | AQDIILQAV (97) | RYLANKIHI (145) |
| DRELRNLEL (2) | NVSSHVHTV (50) | PPRPLGAQV (98) | SLLSGLLRA (146) |
| GARQQIHSW (3) | ALASHLIEA (51) | FNVALNARY (99) | SRIHLVVL (147) |
| SGKLRVAL (4) | ALDDITIQL (52) | GPGSRESTL (100) | SSSPVRGPSV (148) |
| RSASSATQVHK (5) | ALGNTVPAV (53) | IPHQRSSL (101) | STFSLYLKK (149) |
| SASSATQVHK (6) | ALLPAVPSL (54) | LTDRIYLTL (102) | SLDLLPLSI (150) |
| FLLEFKPVS (7) | APAPPPVAV (55) | NLKEKKALF (103) | VTDLLALTV (151) |
| GPQVRGSI (8) | APDKKITL (56) | VLFGGKVSGA (104) | RTQITKVSLKK (152) |
| IRMKAQAL (9) | AQMNLLQKY (57) | VVFPFPVNK (105) | ILRSPLKW (153) |
| KIKVFSKVY (10) | DQVIRLAGL (58) | SLLIIPKKK (106) | LSTGHLSTV (154) |
| LLSRGLLFRI (11) | ETTSQVRKY (59) | APGAAGQRL (107) | TVEEYLVNI (155) |
| LPIASASLL (12) | GGSLIHPQW (60) | KLQDKEIGL (108) | QIKTKLLGSL (156) |
| LYFLGHGSI (13) | GLYYKLHNV (61) | SLREPQPAL (109) | LPSFSHFLLL (157) |
| NPLQLSLSI (14) | GQKPVILTY (62) | TPGRSTQAI (110) | CLRIGPVTL (158) |
| DLMLRESL (15) | GSLDFQRGW (63) | APRGTAAL (111) | HVSDGSTALK (159) |
| VTFKLSLF (16) | HHLVETLKF (64) | IASPIALL (112) | IAYSVRALR (160) |
| IALYKQVL (17) | HLLSETPQL (65) | ILFQNSALK (113) | PRGFLSAL (161) |
| IVATGSLLK (18) | HQLYRASAL (66) | ILKKNISI (114) | ISSWLISSL (162) |
| KlKNKTKNK (19) | HTDDIENAKY (67) | IPLAVRTI (115) | IPLNPFSSL (163) |
| KLLSLTIYK (20) | IAAPILHV (68) | LPRNKPLL (116) | LSDRQLSL (164) |
| NILKKTVL (21) | KAFPFHIIF (69) | PAPPHPAAL (117) | LSHPAPSSL (165) |
| NPKLKDIL (22) | KATEYVHSL (70) | SPVVRVGL (118) | LRKAVDPIL (166) |
| NQKKVRIL (23) | KFSNVTMLF (71) | TLNQGINVYI (119) | ILLEEQSLI (167) |
| PFPLVQVEPV (24) | KLLEKAFSI (72) | RPRGPRTAP (120) | LTSISIRPV (168) |
| SPQSGPAL (25) | KPMPTKVVF (73) | SVQLLEQAIHK (121) | TISECPLLI (169) |
| TSRLPKIQK (26) | NVNRPLTMK (74) | RTPKNYQHW (122) | TLKLKKIFF (170) |
| LLDNILQSI (27) | REPYELTVPAL (75) | ALPVALPSL (123) | ILLSNFSSL (171) |
| RLEVRKVIL (28) | SEAEAAKNAL (76) | SLQILVSSL (124) | LGGAWKAVF (172) |
| LSWGYFLFK (29) | SLWGQPAEA (77) | ISNKVPKLF (125) | LSASHLSSL (173) |
| TILPRILTL (30) | SPADHRGYASL (78) | TVIRIAIVNK (126) | AGDIIARLI (174) |
| EGKIKRNI (31) | SPQSAAAEL (79) | KEIFLELRL (127) | DRGILRNLL (175) |
| FLASFVEKTVL (32) | SPVVHQSL (80) | TLRSPGSSL (128) | GLRLIHVSL (176) |
| ILASHNLTV (33) | SPYRTPVL (81) | TVRGDVSSL (129) | GLRLLHVSL (177) |
| IQLTSVHLL (34) | SVFAGVVGV (82) | ALDPLLLRI (130) | LHNEKGLSL (178) |
| LELISFLPVL (35) | SYSPAHARL (83) | ISLIVTGLK (131) | LPSFSRPSGII (179) |
| NFCMLHQSI (36) | THGSEQLHL (84) | KILDVNLRI (132) | LSSRLPLGK (180) |
| PARPAGPL (37) | TQAPPNVVL (85) | ERVYIRASL (133) | MIGIKRLL (181) |
| PLPIVPAL (38) | VLVPYEPPQV (86) | ILDLESRY (134) | NLKKREIL (182) |
| SNLIRTGSH (39) | VSFPDVRKV (87) | KTFVQQKTL (135) | RMVAYLQQL (183) |
| VPAPAQAI (40) | VVFDKSDLAKY (88) | LYIKSLPAL (136) | SPARALPSL (184) |
| KGHGGPRSW (41) | YSHHSGLEY (89) | VLKEKNASL (137) | TVPGIQRY (185) |
| ITSSAVTTALK (42) | YYLDWIHHY (90) | LGISLTLKY (138) | VSRNYVLLI (186) |
| LLLPESPSI (43) | SVYKYLKAK (91) | DLLPKKLL (139) | LTVPLSVFW (187) |
| VILIPLPPK (44) | IYQFIMDRF (92) | HSLISIVYL (140) | KLNQAFLVL (188) |
| AVLLPKPPK (45) | GTLQGIRAW (93) | IAGALRSVL (141) | RLVSSTLLQK (189) |
| TQVSMAESI (46) | AQKVSVGQAA (94) | IGNPILRVL (142) | LPSHSLLI (190) |
| LNHLRTSI (47) | LYPSKLTHF (95) | IYAPHIRLS (143) | |
| NTSHLPLIY (48) | ATQNTIIGK (96) | LRSQILSY (144) | |

2. The leukemia TAP of item 1, comprising one of the amino acid sequences set forth in SEQ ID NOs: 97-154.

3. The leukemia TAP of item 1 or 2, wherein said leukemia TAP binds to an HLA-A*01:01 molecule and comprises the amino acid sequence NTSHLPLIY (SEQ ID NO:48), HTDDIENAKY (SEQ ID NO:67), YSHHSGLEY (SEQ ID NO:89), ILDLESRY (SEQ ID NO:134), VTDLLALTV (SEQ ID NO:151) or LSDRQLSL (SEQ ID NO:164), preferably ILDLESRY (SEQ ID NO:134) or VTDLLALTV (SEQ ID NO: 151).

4. The leukemia TAP of item 1 or 2, wherein said leukemia TAP binds to an HLA-A*02:01 molecule and comprises the amino acid sequence FLLEFKPVS (SEQ ID NO:7), LLSRGLLFRI (SEQ ID NO:11), LLDNILQSI (SEQ ID NO:27), FLASFVEKTVL (SEQ ID NO:32), ILASHNLTV (SEQ ID NO:33), IQLTSVHLL (SEQ ID NO:34), LELISFLPVL (SEQ ID NO:35), LLLPESPSI (SEQ ID NO:43), ALASHLIEA (SEQ ID NO:51), ALDDITIQL (SEQ ID NO:52), ALGNTVPAV (SEQ ID NO:53), ALLPAVPSL (SEQ ID NO:54), GLYYKLHNV (SEQ ID NO:61), HLLSETPQL (SEQ ID NO:65), KLLEKAFSI (SEQ ID NO:72), SLWGQPAEA (SEQ ID NO:77), SVFAGVVGV (SEQ ID NO:82), VLVPYEPPQV (SEQ ID NO:86), VLFGGKVSGA (SEQ ID NO:104), KLQDKEIGL (SEQ ID NO:108), TLNQGINVYI (SEQ ID NO:119), ALPVALPSL (SEQ ID NO:123), ALDPLLLRI (SEQ ID NO:130), KILDVNLRI (SEQ ID NO:132), SLLSGLLRA (SEQ ID NO:146), SLDLLPLSI (SEQ ID NO:150), ILLEEQSLI (SEQ ID NO:167), LTSISIRPV (SEQ ID NO:168), TISECPLLI (SEQ ID NO:169), ILLSNFSSL (SEQ ID NO:171), RMVAYLQQL (SEQ ID NO:183), or KLNQAFLVL (SEQ ID NO:188), preferably VLFGGKVSGA (SEQ ID NO:104), KLQDKEIGL (SEQ ID NO:108), TLNQGINVYI (SEQ ID NO:119), ALPVALPSL (SEQ ID NO:123), ALDPLLLRI (SEQ ID NO:130), KILDVNLRI (SEQ ID NO:132), SLLSGLLRA (SEQ ID NO:146) or SLDLLPLSI (SEQ ID NO:150).

5. The leukemia TAP of item 1 or 2, wherein said leukemia TAP binds to an HLA-A*03:01 molecule and comprises the amino acid sequence RSASSATQVHK (SEQ ID NO:5), IVATGSLLK (SEQ ID NO:18), KIKNKTKNK (SEQ ID NO:19), KLLSLTIYK (SEQ ID NO:20), ITSSAVTTALK (SEQ ID NO:42), VILIPLPPK (SEQ ID NO:44), NVNRPLTMK (SEQ ID NO:74), SVYKYLKAK (SEQ ID NO:91), VVFPFPVNK (SEQ ID NO:105), ILFQNSALK (SEQ ID NO:113), TVIRIAIVNK (SEQ ID NO:126), ISLIVTGLK (SEQ ID NO:131), HVSDGSTALK (SEQ ID NO:159), IAYSVRALR (SEQ ID NO: 160), LSSRLPLGK (SEQ ID NO: 180) or RLVSSTLLQK (SEQ ID NO: 189), preferably VVFPFPVNK (SEQ ID NO:105), ILFQNSALK (SEQ ID NO:113), TVIRIAIVNK (SEQ ID NO:126) or ISLIVTGLK (SEQ ID NO:131).

6. The leukemia TAP of item 1 or 2, wherein said leukemia TAP binds to an HLA-A*11:01 molecule and comprises the amino acid sequence SASSATQVHK (SEQ ID NO:6), AVLLPKPPK (SEQ ID NO:45), ATQNTIIGK (SEQ ID NO:96), SLLIIPKKK (SEQ ID NO:106), SVQLLEQAIHK (SEQ ID NO:121), STFSLYLKK (SEQ ID NO:149) or RTQITKVSLKK (SEQ ID NO:152), preferably SLLIIPKKK (SEQ ID NO:106), SVQLLEQAIHK (SEQ ID NO:121), STFSLYLKK (SEQ ID NO:149) or RTQITKVSLKK (SEQ ID NO:152).

7. The leukemia TAP of item 1 or 2, wherein said leukemia TAP binds to an HLA-A*24:02 molecule and comprises the amino acid sequence LYFLGHGSI (SEQ ID NO:13), NFCMLHQSI (SEQ ID NO:36), KFSNVTMLF (SEQ ID NO:71), IYQFIMDRF (SEQ ID NO:92), LYPSKLTHF (SEQ ID NO:95) or RYLANKIHI (SEQ ID NO:145), preferably RYLANKIHI (SEQ ID NO:145).

8. The leukemia TAP of item 1 or 2, wherein said leukemia TAP binds to an HLA-A*26:01 molecule and comprises the amino acid sequence ETTSQVRKY (SEQ ID NO:59) or TVPGIQRY (SEQ ID NO: 185).

9. The leukemia TAP of item 1 or 2, wherein said leukemia TAP binds to an HLA-A*29:02 molecule and comprises one of the amino acid sequence VVFDKSDLAKY (SEQ ID NO:88), FNVALNARY (SEQ ID NO:99) or LGISLTLKY (SEQ ID NO:138), preferably FNVALNARY (SEQ ID NO:99) or LGISLTLKY (SEQ ID NO:138).

10. The leukemia TAP of item 1 or 2, wherein said leukemia TAP binds to an HLA-A*30:01 molecule and comprises the amino acid sequence TSRLPKIQK (SEQ ID NO:26), LSWGYFLFK (SEQ ID NO:29) or LSHPAPSSL (SEQ ID NO:165).

11. The leukemia TAP of item 1 or 2, wherein said leukemia TAP binds to an HLA-A*68:02 molecule and comprises the amino acid sequence NVSSHVHTV (SEQ ID NO:50) or SSSPVRGPSV (SEQ ID NO: 148), preferably SSSPVRGPSV (SEQ ID NO: 148).

12. The leukemia TAP of item 1 or 2, wherein said leukemia TAP binds to an HLA-B*07:02 molecule and comprises the amino acid sequence GPQVRGSI (SEQ ID NO:8), SPQSGPAL (SEQ ID NO:25), VPAPAQAI (SEQ ID NO:40), APAPPPVAV (SEQ ID NO:55), APDKKITL (SEQ ID NO:56), KPMPTKVVF (SEQ ID NO:73), SPADHRGYASL (SEQ ID NO:78), SPQSAAAEL (SEQ ID NO:79), SPWHQSL (SEQ ID NO:80), SPYRTPVL (SEQ ID NO:81), PPRPLGAQV (SEQ ID NO:98), GPGSRESTL (SEQ ID NO: 100), APGAAGQRL (SEQ ID NO:107), TPGRSTQAI (SEQ ID NO:110), APRGTAAL (SEQ ID NO:111), SPVVRVGL (SEQ ID NO:118), RPRGPRTAP (SEQ ID NO:120), TLRSPGSSL (SEQ ID NO:128), TVRGDVSSL (SEQ ID NO:129), LPSFSHFLLL (SEQ ID NO:157), PRGFLSAL (SEQ ID NO:161), IPLNPFSSL (SEQ ID NO:163), LPSFSRPSGII (SEQ ID NO:179) or SPARALPSL (SEQ ID NO:184), preferably PPRPLGAQV (SEQ ID NO:98), GPGSRESTL (SEQ ID NO: 100), APGAAGQRL (SEQ ID NO:107), TPGRSTQAI (SEQ ID NO:110), APRGTAAL (SEQ ID NO:111), SPVVRVGL (SEQ ID NO:118), RPRGPRTAP (SEQ ID NO:120), TLRSPGSSL (SEQ ID NO:128) or TVRGDVSSL (SEQ ID NO:129).

13. The leukemia TAP of item 1 or 2, wherein said leukemia TAP binds to an HLA-B*08:01 molecule and comprises the amino acid sequence SGKLRVAL (SEQ ID NO:4), NPLQLSLSI (SEQ ID NO:14), DLMLRESL (SEQ ID NO:15), IALYKQVL (SEQ ID NO:17), NILKKTVL (SEQ ID NO:21), NPKLKDIL (SEQ ID NO:22), NQKKVRIL (SEQ ID NO:23), RLEVRKVIL (SEQ ID NO:28), EGKIKRNI (SEQ ID NO:31), LNHLRTSI (SEQ ID NO:47), SIQRNLSL (SEQ ID NO:49), IPHQRSSL (SEQ ID NO:101), NLKEKKALF (SEQ ID NO:103), ILKKNISI (SEQ ID NO:114), VLKEKNASL (SEQ ID NO:137), DLLPKKLL (SEQ ID NO:139), SRIHLWL (SEQ ID NO:147), QIKTKLLGSL (SEQ ID NO:156), TLKLKKIFF (SEQ ID NO:170), MIGIKRLL (SEQ ID NO:181) or NLKKREIL (SEQ ID NO:182), preferably IPHQRSSL (SEQ ID NO:101), NLKEKKALF (SEQ ID NO:103), ILKKNISI (SEQ ID NO:114), VLKEKNASL (SEQ ID NO:137), DLLPKKLL (SEQ ID NO:139) or SRIHLWL (SEQ ID NO:147).

14. The leukemia TAP of item 1 or 2, wherein said leukemia TAP binds to an HLA-B*14:01 molecule and comprises the amino acid sequence DRELRNLEL (SEQ ID NO:2), SNLIRTGSH (SEQ ID NO:39), DQVIRLAGL (SEQ ID NO:58), HQLYRASAL (SEQ ID NO:66), SLQILVSSL (SEQ ID NO:124), ERVYIRASL (SEQ ID NO:133), LYIKSLPAL (SEQ ID NO:136), IAGALRSVL (SEQ ID NO:141), ISSWLISSL (SEQ ID NO:162), DRGILRNLL (SEQ ID NO:175), GLRLIHVSL (SEQ ID NO:176) or GLRLLHVSL (SEQ ID NO:177), preferably SLQILVSSL (SEQ ID NO:124), ERVYIRASL (SEQ ID NO: 133), LYIKSLPAL (SEQ ID NO: 136) or IAGALRSVL (SEQ ID NO: 141).

15. The leukemia TAP of item 1 or 2, wherein said leukemia TAP binds to an HLA-B*15:01 molecule and comprises the amino acid sequence KIKVFSKVY (SEQ ID NO:10), AQMNLLQKY (SEQ ID NO:57), GQKPVILTY (SEQ ID NO:62) or AQKVSVGQAA (SEQ ID NO:94).

16. The leukemia TAP of item 1 or 2, wherein said leukemia TAP binds to an HLA-B*27:05 molecule and comprises the amino acid sequence RQISVQASL (SEQ ID NO:1) or LRSQILSY (SEQ ID NO:144), preferably LRSQILSY (SEQ ID NO:144).

17. The leukemia TAP of item 1 or 2, wherein said leukemia TAP binds to an HLA-B*38:01 molecule and comprises the amino acid sequence TQVSMAESI (SEQ ID NO:46), HHLVETLKF (SEQ ID NO:64) or THGSEQLHL (SEQ ID NO:84).

18. The leukemia TAP of item 1 or 2, wherein said leukemia TAP binds to an HLA-B*40:01 molecule and comprises the amino acid sequence REPYELTVPAL (SEQ ID NO:75) or SEAEAAKNAL (SEQ ID NO:76).

19. The leukemia TAP of item 1 or 2, wherein said leukemia TAP binds to an HLA-B*44:03 molecule and comprises the amino acid sequence KEIFLELRL (SEQ ID NO:127).

20. The leukemia TAP of item 1 or 2, wherein said leukemia TAP binds to an HLA-B*51:01 molecule and comprises the amino acid sequence LPIASASLL (SEQ ID NO:12), PFPLVQVEPV (SEQ ID NO:24), PLPIVPAL (SEQ ID NO:38), IAAPILHV (SEQ ID NO:68), IPLAVRTI (SEQ ID NO: 115), LPRNKPLL (SEQ ID NO: 116) or LPSHSLLI (SEQ ID NO: 190), preferably IPLAVRTI (SEQ ID NO:115) or LPRNKPLL (SEQ ID NO:116).

21. The leukemia TAP of item 1 or 2, wherein said leukemia TAP binds to an HLA-B*57:01 molecule and comprises the amino acid sequence GARQQIHSW (SEQ ID NO:3), VTFKLSLF (SEQ ID NO:16), KGHGGPRSW (SEQ ID NO:41), GSLDFQRGW (SEQ ID NO:63), KAFPFHIIF (SEQ ID NO:69), GTLQGIRAW (SEQ ID NO:93), RTPKNYQHW (SEQ ID NO:122), ISNKVPKLF (SEQ ID NO:125), KTFVQQKTL (SEQ ID NO:135), ILRSPLKW (SEQ ID NO:153) or LTVPLSVFW (SEQ ID NO:183), preferably RTPKNYQHW (SEQ ID NO:122), ISNKVPKLF (SEQ ID NO:125), KTFVQQKTL (SEQ ID NO:135) or ILRSPLKW (SEQ ID NO:153).

22. The leukemia TAP of item 1 or 2, wherein said leukemia TAP binds to an HLA-B*57:03 molecule and comprises the amino acid sequence GGSLIHPQW (SEQ ID NO:60) or LGGAWKAVF (SEQ ID NO:172).

23. The leukemia TAP of item 1 or 2, wherein said leukemia TAP binds to an HLA-C*03:03 molecule and comprises the amino acid sequence PARPAGPL (SEQ ID NO:37), IASPIALL (SEQ ID NO:112) or HSLISIVYL (SEQ ID NO:140), preferably IASPIALL (SEQ ID NO:112) or HSLISIVYL (SEQ ID NO:140).

24. The leukemia TAP of item 1 or 2, wherein said leukemia TAP binds to an HLA-C*05:01 molecule and comprises the amino acid sequence SLDLLPLSI (SEQ ID NO:150).

25. The leukemia TAP of item 1 or 2, wherein said leukemia TAP binds to an HLA-C*06:02 molecule and comprises the amino acid sequence IRMKAQAL (SEQ ID NO:9), KATEYVHSL (SEQ ID NO:70), VSFPDVRKV (SEQ ID NO:87), IGNPILRVL (SEQ ID NO:142), LSTGHLSTV (SEQ ID NO:154) or LRKAVDPIL (SEQ ID NO:166), preferably IGNPILRVL (SEQ ID NO:142) or LSTGHLSTV (SEQ ID NO:154).

26. The leukemia TAP of item 1 or 2, wherein said leukemia TAP binds to an HLA-C*07:01 molecule and comprises the amino acid sequence IGNPILRVL (SEQ ID NO:142), IYAPHIRLS (SEQ ID NO:143), TVEEYLVNI (SEQ ID NO:155), LHNEKGLSL (SEQ ID NO:178) or VSRNYVLLI (SEQ ID NO:186), preferably IGNPILRVL (SEQ ID NO:142) or IYAPHIRLS (SEQ ID NO:143).

27. The leukemia TAP of item 1 or 2, wherein said leukemia TAP binds to an HLA-C*07:02 molecule and comprises the amino acid sequence TILPRILTL (SEQ ID NO:30), SYSPAHARL (SEQ ID NO:83), TQAPPNWL (SEQ ID NO:85), YYLDWIHHY (SEQ ID NO:90), SLREPQPAL (SEQ ID NO:109), PAPPHPAAL (SEQ ID NO:117) or CLRIGPVTL (SEQ ID NO:158), preferably SLREPQPAL (SEQ ID NO:109) or PAPPHPAAL (SEQ ID NO:117).

28. The leukemia TAP of item 1 or 2, wherein said leukemia TAP binds to an HLA-C*08:02 molecule and comprises the amino acid sequence AQDIILQAV (SEQ ID NO:97), LTDRIYLTL (SEQ ID NO:102) or AGDIIARLI (SEQ ID NO:174), preferably AQDIILQAV (SEQ ID NO:97) or LTDRIYLTL (SEQ ID NO:102).

29. The leukemia TAP of item 1 or 2, wherein said leukemia TAP binds to an HLA-C*12:03 molecule and comprises the amino acid sequence LSASHLSSL (SEQ ID NO:173).

30. The leukemia TAP of any one of items 1-29, which is encoded by a sequence located a non-protein coding region of the genome.

31. The leukemia TAP of item 30, wherein said non-protein coding region of the genome is an untranslated transcribed region (UTR).

32. The leukemia TAP of item 30, wherein said non-protein coding region of the genome is an intron.

33. The leukemia TAP of item 30, wherein said non-protein coding region of the genome is an intergenic region.

34. A combination comprising at least two of the leukemia TAPs defined in any one of items 1-33

35. A nucleic acid encoding the leukemia TAP of any one of items 1-33 or the combination of item 34.

36. The nucleic acid of item 35, which is an mRNA or a viral vector.

37. A liposome comprising the leukemia TAP of any one of items 1-33, the combination of item 34, or the nucleic acid of item 35 or 36.

38. A composition comprising the leukemia TAP of any one of items 1-33, the combination of item 34, the nucleic acid of item 35 or 36, or the liposomes of item 37, and a pharmaceutically acceptable carrier.

39. A vaccine comprising the leukemia TAP of any one of items 1-33, the combination of item 34, the nucleic acid of item 35 or 36, the liposomes of item 37, or the composition of item 38, and an adjuvant.

40. An isolated major histocompatibility complex (MHC) class I molecule comprising the leukemia TAP of any one of items 1-33 in its peptide binding groove.

41. The isolated MHC class I molecule of item 40, which is in the form of a multimer.

42. The isolated MHC class I molecule of item 41, wherein said multimer is a tetramer.

43. An isolated cell comprising (i) the leukemia TAP of any one of items 1-33, (ii) the combination of item 34 or (iii) a vector comprising a nucleotide sequence encoding TAP of any one of items 1-33 or the combination of item 34.

44. An isolated cell expressing at its surface major histocompatibility complex (MHC) class I molecules comprising the leukemia TAP of any one of items 1-33 or the combination of item 34 in their peptide binding groove.

45. The cell of item 44, which is an antigen-presenting cell (APC).

46. The cell of item 45, wherein said APC is a dendritic cell.

47. A T-cell receptor (TCR) that specifically recognizes the isolated MHC class I molecule of any one of items 40-42 and/or MHC class I molecules expressed at the surface of the cell of any one of items 44-46.

48. The TCR of item 47, wherein said TCR comprises a TCRbeta (TCRβ) chain comprising a complementary determining region 3 (CDR3) comprising one of the amino acid sequences set forth in SEQ ID NO: 191-219.

49. An isolated cell expressing at its cell surface the TCR of item 47 or 48.

50. The isolated cell of item 49, which is a CD8⁺ T lymphocyte.

51. A cell population comprising at least 0.5% of the isolated cell as defined in item 49 or 50.

52. A method of treating leukemia in a subject comprising administering to the subject an effective amount of: (i) the leukemia TAP of any one of items 1-33; (ii) the combination of item 34; (iii) the nucleic acid of item 35 or 36; (iv) the liposome of item 37; (v) the composition of item 38; (vi) the vaccine of item 39; (vii) the cell of any one of items 43-46, 49 and 50; or (viii) the cell population of item 51.

53. The method of item 52, wherein said leukemia is a myeloid leukemia.

54. The method of item 53, wherein said myeloid leukemia is acute myeloid leukemia (AML).

55. The method of any one of items 52-54, further comprising administering at least one additional antitumor agent or therapy to the subject.

56. The method of item 55, wherein said at least one additional antitumor agent or therapy is a chemotherapeutic agent, immunotherapy, an immune checkpoint inhibitor, radiotherapy or surgery.

57. Use of: (i) the leukemia TAP of any one of items 1-33; (ii) the combination of item 34; (iii) the nucleic acid of item 35 or 36; (iv) the liposome of item 37; (v) the composition of item 38; (vi) the vaccine of item 39; (vii) the cell of any one of items 43-46, 49 and 50; or (viii) the cell population of item 51, for treating leukemia in a subject.

58. Use of: (i) the leukemia TAP of any one of items 1-33; (ii) the combination of item 34; (iii) the nucleic acid of item 35 or 36; (iv) the liposome of item 37; (v) the composition of item 38; (vi) the vaccine of item 39; (vii) the cell of any one of items 43-46, 49 and 50; or (viii) the cell population of item 51, for the manufacture of a medicament for treating leukemia in a subject.

59. The use of item 57 or 58, wherein said leukemia is a myeloid leukemia.

60. The use of item 59, wherein said myeloid leukemia is acute myeloid leukemia (AML).

61. The use of any one of items 57-60, further comprising the use of at least one additional antitumor agent or therapy.

62. The use of item 61, wherein said at least one additional antitumor agent or therapy is a chemotherapeutic agent, immunotherapy, an immune checkpoint inhibitor, radiotherapy or surgery.

63. The: (i) leukemia TAP of any one of items 1-33; (ii) combination of item 34; (iii) nucleic acid of item 35 or 36; (iv) liposome of item 37; (v) composition of item 38; (vi) vaccine of item 39; (vii) cell of any one of items 43-46, 49 and 50; or (viii) cell population of item 51, for use in the treatment of leukemia in a subject.

64. The leukemia TAP, combination, nucleic acid, liposome, composition, vaccine, cell or cell population for use according to item 63, wherein said leukemia is a myeloid leukemia.

65. The leukemia TAP, combination, nucleic acid, liposome, composition, vaccine, cell or cell population for use according to item 64, wherein said myeloid leukemia is acute myeloid leukemia (AML).

66. The leukemia TAP, combination, nucleic acid, liposome, composition, vaccine, cell or cell population for use according to any one of items 63-65, which is for use in combination with at least one additional antitumor agent or therapy.

67. The leukemia TAP, combination, nucleic acid, liposome, composition, vaccine, cell or cell population for use according to item 66, wherein said at least one additional antitumor agent or therapy is a chemotherapeutic agent, immunotherapy, an immune checkpoint inhibitor, radiotherapy or surgery.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIGS. 1A-D are graphs showing that hematopoietic progenitors are better controls than mTECs to discover TSAs in AML. FIG. 1A: Comparison of the efficacy of k-mer depletion from the k-mer set of each of the 19 AML specimens by either the combined k-mers from the 6 mTEC or from 6 MPC samples. K-mers of occurrence < 2 were ignored and jellyfish databases were generated in canonical mode for this comparison. FIG. 1B: Overlap between combined k-mers of all AML specimens and k-mers from the 6 mTECs and from 6 MPCs samples used in FIG. 1A. Parameters for database construction used in FIG. 1A were re-applied here. FIG. 1C: T-distributed Stochastic Neighbor Embedding (t-SNE) analyses of protein coding genes expressed (TPM ≥ 1) in purified cell populations from indicated tissues. FIG. 1D: Comparison of the total number of expressed protein coding genes (TPM ≥ 1) in indicated tissues and cell populations used to plot panel C. Pluri_stem: Pluripotent stem cells; Ery: Erythroid; Precu: Precursor; Lympho: Lymphocytic; Granulo: Granulocytic; Mono: Monocytic. Mann whitney U test was used to compare mTECs with each other tissue (****p<0.0001), bars show average with standard deviation.

FIG. 2 depicts schematic overviews of MPC-based TSA discovery approaches. (A) Schematic overview of the workflow for TSA discovery based on mTEC k-mers depletion. (B) Schematic overview of the workflow for ERE-derived MAPs discovery. (C) Schematic overview of the workflow for mTECs+MPCs k-mers depletion TSA discovery approach. (D) Schematic overview of the workflow for the DKE approach. Illustrated here is the workflow for AML#1 sample. A fold-change of 10 was used as minimum to consider a k-mer as overexpressed (other filters were also applied, see methods). As for the three other approaches, the obtained database of in silica all-frame translated contigs was concatenated with a personalized canonical proteome before performing MS identifications of MAPs eluted from the same AML samples used to perform RNA sequencing.

FIGS. 3A-H show that MPCs-based approaches identify the majority of TSA^{hi} in AML. σ) of the distribution (plotted in black) are given. FIG. 3A: Proportion of MAPs, for each AML specimen (n=19), deriving from transcripts segregated in 10 different groups (deciles) based on their TPM expression. Decile 10 has transcripts with the greatest expression and decile 1 with the lowest. Boxes show the median, 25 and 75th percentiles of the distribution and whiskers extend to the minimum and maximum. FIG. 3B: Normal distribution of the cumulative frequency of MAPs (dots) in function of the log of total number of RNA-seq reads capable of coding for them (rphm) in the AML specimen from which they were identified by MS. Average (µ) and standard deviation (σ) of the distribution (plotted in black) are given. FIG. 3C: Probability (computed based on normal distribution parameters of FIG. 3B for an RNA sequence to generate a MAP after the different indicated fold-changes (FC, original rphm × FC). FIG. 3D: Decision tree used to segregate MAPs-of-interest (MOI) into TAAs, HSAs, TSAs^{hi}. “Normal tissues” refers to all tissues (GTEx, purified hematopoietic cells and mTECs) and Blood/BM refers only to purified hematopoietic cells. FIG. 3E: Comparison of MOI counts obtained by each indicated proteogenomic approaches. FIG. 3F: Venn diagram comparing TSAs^{hi} identity between the indicated approaches. FIG. 3G: Pearson correlations between observed retention times and predicted retention time (left) or hydrophobicity index (right). FIG. 3H: Median and interquartile range frequency of successful re-identification of indicated MAPs with Comet.

FIGS. 4A-K show TSAs^{hi} derive mainly from intron translation and are shared among many patients. FIG. 4A: Heatmap depicting the average RNA expression (log of rphm +1) of each identified TSA^{hi} in either total normal tissues from GTEx (n=12-50 depending on available samples), normal sorted hematopoietic cell populations (n=3-16 depending on available samples) or in mTECs (n=11). TAAs evaluated as safe in clinical trials are also reported. Prec: precursor. FIG. 4B: Comparison of TSAs^{hi} fold changes between the average rphm expression in the 19 AML specimens and MPCs (n=16). Dots show each MOI, boxes show the median, 25 and 75th percentiles of the distribution and whiskers extend to the minimum and maximum. FIG. 4C: Distributions of biotypes (genomic region or event) having generated the indicated MOIs. Exon-intron: peptides overlapping an exon-intron junction (retained intron); ncRNA: non-coding RNA; OoF translation: out-of-frame translation. FIG. 4D: TSAs^{hi} RNA expression in the 19 AML samples and in the 437 Leucegene patients. FIG. 4E: Population coverage by the HLA allotypes capable of presenting TSAs^{hi} (19 AML specimen alleles presenting the TSAs^{hi} + promiscuous binders computed by MHCcluster). This was calculated with the IEDB population coverage tool (www.iedb.org). Bars indicate the frequencies of individuals within the world population carrying up to six allotypes (x-axis) and cumulative percentage of population coverage is shown as dots. FIG. 4F: HLA-TSA^{hi} complex distribution in the Leucegene cohort based on TSAs^{hi} RNA expression (considered expressed if rphm ≥ 2), HLA alleles of patients (OptiType) and promiscuous binders. The distinction between high (upper quartile) and low (all other patients) TSAs^{hi} expressors is shown. FIG. 4G: #_{pred}HLA-TSA^{hi} complexes in Leucegene patients at diagnosis and relapse. FIG. 4H: RNA expression of TSAs^{hi} that could be presented by HLA alleles of AML blasts pairwise-purified from 15 patients at time of diagnosis and at relapse (data from (Toffalori et al., 2019)). Comparison made with the Wilcoxon matched-pairs signed rank test.. FIG. 4I: Comparison of TSAs^{hi} inter-sample sharing (considered expressed if rphm>0) in sorted blasts (n=12) or leukemia stem cells (LSCs, n=8), reported elsewhere (Corces et al., 2016). FIG. 4J: RNA expression of HLA-ABC molecules in samples shown in FIG. 4I. Average +SD are shown. FIG. 4K: GSEA analysis comparing Leucegene patients expressing (rphm>0) ≥ median numbers of TSAs^{hi} (n=207) vs the others (n=230) for the indicated LSC signature gene set (Eppert et al. 2011). NES, normalized enrichment score.

FIGS. 5A-F show that presentation of numerous TSAs^{hi} correlates with better survival. FIG. 5A: Kaplan-Meier survival analysis between Leucegene patients expressing high (n=98, upper quartile in FIG. 5B) vs. low numbers (n=275, all other patients) of HLA-TSAs^{hi} complexes. Statistical significance was determined by the log-rank test. FIG. 5B: Forest plot for multivariable analyses of 5-year overall survival. HR, adjusted hazard ratio; Cl, confidence intervals; adv, adverse; fav, favorable; int, intermediate. NPM1 / FLT3 interaction = presence of both NPM1^{mut} and FLT3-ITD. FIG. 5C: Log-rank p-values computed after removal of indicated number of TSAs^{hi} from the analysis performed in (A); 1000 permutations were made for each number and average +SD are reported. FIG. 5D: Percentage of significant p-values obtained in FIG. 5C. FIG. 5E: Comparison of log-rank p-values re-computed after the alternative removal of each TSA^{hi} from the analysis in FIG. 5A. FIG. 5F: Comparison of log-rank p-values re-computed after the alternative removal of each HLA allele from the analysis in FIG. 5A.

FIGS. 6A-O show that TSAs^{hi} presentation triggers cytotoxic T cell responses. FIG. 6A: Comparison of immunogenicity scores (Repitope) between MOIs, MAPs from thymic stromal cells and HIV MAPs. FIG. 6B: Median and interquartile range of average RNA expression across 11 available mTEC samples for MOIs, 5112 non-immunogenic MAPs and 1411 immunogenic MAPs (from IEDB and curated in (Ogishi and Yotsuyanagi, 2019)). FIG. 6C: IFN-y ELISpot assay of healthy PBMCs after stimulation with DCs pulsed with indicated peptides. Results from 2 independent experiments were combined. FIG. 6D: ELISpot assays of indicated TSAs^{hi} (single donor). FIG. 6E: Flow cytometry analysis of cytokine secretion of T cells expanded in presence of indicated peptides. FIG. 6F: Representative flow cytometry plots of indicated dextramer frequency among T cells expanded in presence of indicated peptides. FIG. 6G: FEST assay: expansion of significant T-cell clonotypes after 10 days of stimulation with 3 different pools of TSAs^{hi} (5 peptides / pool). FIG. 6H: TCR CDR3s per thousand TCR reads (CPK, as measure of clonotype diversity) in Leucegene patients having high vs low counts of indicated _{pred}HLA-MOIs (related to FIGS. 4F and 11D). FIG. 6I: Frequency of clonotypes predicted by ERGO to react against TSAs^{hi} (n = 66-164 / group) in Leucegene. FIG. 6J: Frequency of clonotypes predicted by ERGO to react against TAAs (n = 74-207 / group) in Leucegene. FIG. 6K: Frequency of clonotypes recognizing MOIs _{pred}presented in the considered sample among all anti-MOIs clonotypes, normalized by the number of _{pred}presented MOIs (related to I and J). Patients having anti-ₚᵣₑₛMOIs clonotype counts = 0 were ignored. FIG. 6L: Correlation between the RNA expression of CD8A and CD8B genes and the number of TSAs^{hi} expressed above 2 rphm in Leucegene. FIG. 6M: Correlation between the RNA expression of CD8A and CD8B genes and the number of _{pred}HLA-TSAs^{hi} in Leucegene. FIG. 6N: Volcano plot of differential gene expression analysis comparing patients whose normalized TSAs^{hi} _{pred}presentation was above- vs. below-median. Dots show genes upregulated in above-median patients. FIG. 6O: GO term analysis of upregulated genes in FIG. 6N.

FIGS. 7A-G show that TSAs^{hi} expression is associated with immunoediting, AML driver mutations and epigenetic aberrations. FIG. 7A: Pearson correlations between the number of HE-TSAs^{hi} and the expression of indicated genes across the full Leucegene cohort (n=437). Expression values of HLA-A, -B and -C were summed for the first panel. FIG. 7B: Comparison of the PD-L1 (CD274) gene expression between Leucegene patients expressing ≥ median numbers of HE-TSAs^{hi}vs the others, stratified as a function of NPM1 mutational status. FIG. 7C: Network analysis of GO term enrichment among genes inversely correlated with HE-TSAs^{hi} numbers. Node size is proportional to gene set size. FIG. 7D: Network analysis of GO term enrichment among genes positively correlated with HE-TSAs^{hi} numbers. FIG. 7E: Comparison of patient numbers expressing ≥ median numbers of HE-TSAs^{hi} vs the others among WT and mutant patients for indicated genes. Statistical significance established with the Fisher’s exact test (**p<0.01, ****p<0.0001). FIG. 7F: Comparison of HE-TSAs^{hi} numbers between patients having 0 to 3 mutations in either NPM1, FLT3 or DNMT3A. FIG. 7G: Unsupervised consensus clustering for intron retention ratios, as determined by IRFinder, for Leucegene patients (n=437, columns). Rows represent the 1211 top-ranked introns of highest variability and significance for consensus clustering, clustered hierarchically. FAB types of patients are shown below the heatmap with p-values (Fisher’s exact tests, *p<0.05, **p<0.01, ***p<0.001, ****p<0.0001) shown for significant associations with indicated consensus clusters.

FIG. 8A is an illustration of the concept of k-mer occurrence.

FIG. 8B is a graph depicting an example of k-mer frequency distribution in function of occurrence in sample 05H143.

FIG. 8C is a graph depicting a comparison of threshold occurrences used between mTECs only and mTECs+MPCs k-mer depletion approaches (each dot is a different AML sample).

FIG. 8D is a graph depicting the overlap of k-mer identity between the combination of unique k-mers obtained from all 19 AML specimens obtained after depletion of either mTECs or mTECs+MPCs k-mers.

FIG. 9A is a schematic providing the details of the differential k-mer expression analysis and MS database building. The building of MS database for sample AML#1 is presented as example. FC, Fold Change, is a diagram showing the details of the differential k-mer expression analysis and MS database building. The building of MS database for sample AML#1 is presented as example. FC, Fold-Change.

FIG. 9B is a graph depicting the cumulative number of canonical peptide identifications (peptides deriving from the personalized canonical proteome, either alone (Canon.) or concatenated with contigs sequences in the four indicated approaches) vs the average database size (line).

FIG. 9C depicts Venn diagrams comparing the overlap of identity of canonical peptides identified based on each approach with peptides identified based on the canonical personalized proteome alone.

FIG. 10A is a graph depicting a comparison of the proportion of MHC-I-associated peptides (MAPs) of interest (MOIs), identified by each TSA-identification approach.

FIG. 10B is a graph depicting a comparison of the total number of AML specimens (out of the 19 used to identify TSAs in the present study) expressing (rphm > 0) TSA^{hi} identified with either the mTECs + MPCs k-mer depletion or with the differential k-mer expression approaches.

FIG. 11A is a graph showing the distribution of numbers of TSAs^{hi} having an RNA expression equal or higher than 2 rphm in the Leucegene cohort (n=437).

FIG. 11B is a graph showing the survival comparison between patients of the Leucegene cohort (n=372 sequenced at diagnosis and for which survival data were available) presenting high numbers of TSA^{hi} expressed at levels equal or higher than 2 rphm (upper quartile of the distribution in left panel) vs those presenting low levels (rest of the cohort).

FIGS. 11C-E are graphs showing the HLA-MOI complex distribution across the whole Leucegene cohort obtained based on RNA expression (considered expressed if rphm ≥ 2), HLA alleles of each patient and promiscuous binders prediction (optitype and MHCcluster) for HSAs (FIG. 11C), TAAs (FIG. 11D) and TSAs^{lo}(FIG. 11E).

FIGS. 11F-H are graphs showing the survival comparison between patients of the Leucegene cohort (n=372 sequenced at diagnosis and for which survival data were available) presenting high numbers of HLA-MOI complexes (upper quartile of the distribution in top panels) vs those presenting low levels (rest of the cohort) for HSAs (FIG. 11F), TAAs (FIG. 11G) and TSAs^{lo} (FIG. 11H).

FIG. 12A depicts Pearson correlations between the number of HE-TSAs^{hi} and the expression of indicated genes across the full Leucegene cohort (n=437).

FIG. 12B depicts graphs showing a comparison of indicated genes expression in patients having high _{pred}presentation level of TSAs^{hi} vs the rest of the patients (related to FIG. 4F).

FIG. 12C depicts Pearson correlations between the expression of ZNF445 and the number of retained introns in the Leucegene cohort (analyzed with IRFinder and defined as retained if retained in more than 10% of transcripts).

FIG. 12D is a graph showing a comparison of patients expressing ≥ median numbers of HE-TSAs^{hi} vs the others among WT and mutant patients for indicated genes. Statistical significance established with the Fisher’s exact test.

FIG. 12E is a graph showing a comparison of patients expressing ≥ median numbers of HE-TSAs^{hi} vs the others among patients receiving allo-HSCT or not. Statistical significance established with the Fisher’s exact test.

FIG. 12F is a graph showing the FAB types distribution of patients having counts of HE-TSAs^{hi} above or below the median HE-TSAs^{hi} count across the whole Leucegene cohort.

FIG. 12G is a graph showing the WHO 2008 classification distribution of patients having counts of HE-TSAs^{hi} above or below the median HE-TSAs^{hi} count across the whole Leucegene cohort.

FIG. 12H is a graph showing the cytogenetic profiles distribution of patients having counts of HE-TSAs^{hi} above or below the median HE-TSAs^{hi} count across the whole Leucegene cohort.

### DISCLOSURE OF INVENTION

Terms and symbols of genetics, molecular biology, biochemistry and nucleic acid used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like. All terms are to be understood with their typical meanings established in the relevant art.

The articles “a” and “an” are used herein to refer to one or to more than one (i.e. to at least one) of the grammatical object of the article. By way of example, “an element” means one element or more than one element. Throughout this specification, unless the context requires otherwise, the words “comprise,” “comprises” and “comprising” will be understood to imply the inclusion of a stated step or element or group of steps or elements but not the exclusion of any other step or element or group of steps or elements.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

All methods described herein can be performed in any suitable order unless otherwise indicated herein or otherwise clearly contradicted by context.

The use of any and all examples, or exemplary language (e.g., “such as”) provided herein, is intended merely to better illustrate the invention and does not pose a limitation on the scope of the invention unless otherwise claimed.

No language in the specification should be construed as indicating any non-claimed element as essential to the practice of the invention.

Herein, the term “about” has its ordinary meaning. The term “about” is used to indicate that a value includes an inherent variation of error for the device or the method being employed to determine the value, or encompass values close to the recited values, for example within 10% or 5% of the recited values (or range of values).

In the studies described herein, the present inventors have identified TSA candidates from 19 AML specimens using a proteogenomic-based approach. A large fraction of these TSAs derived from aberrantly expressed unmutated genomic sequences which are not expressed in normal tissues, such as non-exonic sequences (e.g., intronic and intergenic sequences). The expression of these AML TSA candidates was shown to correlate with mutations of epigenetic modifiers (e.g., DNMT3A) and with expression of ZNF445 a regulator of genomic imprinting. It is also shown that the AML TSA candidates are highly shared among patients, are expressed in both blasts and leukemic stem cells, and their HLA presentation is associated with markers of immunoediting and better overall survival. Thus, the novel AML TSA candidates identified herein may be useful for leukemia T-cell based immunotherapy.

Accordingly, in an aspect, the present disclosure relates to a leukemia TAP (or leukemia tumor-specific peptide) comprising, or consisting of, one of the following amino acid sequences:

| | | | |
|---|---|---|---|
| Sequence (SEQ ID NO) | Sequence (SEQ ID NO) | Sequence (SEQ ID NO) | Sequence (SEQ ID NO.) |
| RQISVQASL (1) | SIQRNLSL (49) | AQDIILQAV (97) | RYLANKIHI (145) |
| DRELRNLEL (2) | NVSSHVHTV (50) | PPRPLGAQV (98) | SLLSGLLRA (146) |
| GARQQIHSW (3) | ALASHLIEA (51) | FNVALNARY (99) | SRIHLVVL (147) |
| SGKLRVAL (4) | ALDDITIQL (52) | GPGSRESTL (100) | SSSPVRGPSV (148) |
| RSASSATQVHK (5) | ALGNTVPAV (53) | IPHQRSSL (101) | STFSLYLKK (149) |
| SASSATQVHK (6) | ALLPAVPSL (54) | LTDRIYLTL (102) | SLDLLPLSI (150) |
| FLLEFKPVS (7) | APAPPPVAV (55) | NLKEKKALF (103) | VTDLLALTV (151) |
| GPQVRGSI (8) | APDKKITL (56) | VLFGGKVSGA (104) | RTQITKVSLKK (152) |
| IRMKAQAL (9) | AQMNLLQKY (57) | VVFPFPVNK (105) | ILRSPLKW (153) |
| KIKVFSKVY (10) | DQVIRLAGL (58) | SLLIIPKKK (106) | LSTGHLSTV (154) |
| LLSRGLLFRI (11) | ETTSQVRKY (59) | APGAAGQRL (107) | TVEEYLVNI (155) |
| LPIASASLL (12) | GGSLIHPQW (60) | KLQDKEIGL (108) | QIKTKLLGSL (156) |
| LYFLGHGSI (13) | GLYYKLHNV (61) | SLREPQPAL (109) | LPSFSHFLLL (157) |
| NPLQLSLSI (14) | GQKPVILTY (62) | TPGRSTQAI (110) | CLRIGPVTL (158) |
| DLMLRESL (15) | GSLDFQRGW (63) | APRGTAAL (111) | HVSDGSTALK (159) |
| VTFKLSLF (16) | HHLVETLKF (64) | IASPIALL (112) | IAYSVRALR (160) |
| IALYKQVL (17) | HLLSETPQL (65) | ILFQNSALK (113) | PRGFLSAL (161) |
| IVATGSLLK (18) | HQLYRASAL (66) | ILKKNISI (114) | ISSWLISSL (162) |
| KIKNKTKNK (19) | HTDDIENAKY (67) | IPLAVRTI (115) | IPLNPFSSL (163) |
| KLLSLTIYK (20) | IAAPILHV (68) | LPRNKPLL (116) | LSDRQLSL (164) |
| NILKKTVL (21) | KAFPFHIIF (69) | PAPPHPAAL (117) | LSHPAPSSL (165) |
| NPKLKDIL (22) | KATEYVHSL (70) | SPVVRVGL (118) | LRKAVDPIL (166) |
| NQKKVRIL (23) | KFSNVTMLF (71) | TLNQGINVYI (119) | ILLEEQSLI (167) |
| PFPLVQVEPV (24) | KLLEKAFSI (72) | RPRGPRTAP (120) | LTSISIRPV (168) |
| SPQSGPAL (25) | KPMPTKVVF (73) | SVQLLEQAIHK (121) | TISECPLLI (169) |
| TSRLPKIQK (26) | NVNRPLTMK (74) | RTPKNYQHW (122) | TLKLKKIFF (170) |
| LLDNILQSI (27) | REPYELTVPAL (75) | ALPVALPSL (123) | ILLSNFSSL (171) |
| RLEVRKVIL (28) | SEAEAAKNAL (76) | SLQILVSSL (124) | LGGAWKAVF (172) |
| LSWGYFLFK (29) | SLWGQPAEA (77) | ISNKVPKLF (125) | LSASHLSSL (173) |
| TILPRILTL (30) | SPADHRGYASL (78) | TVIRIAIVNK (126) | AGDIIARLI (174) |
| EGKIKRNI (31) | SPQSAAAEL (79) | KEIFLELRL (127) | DRGILRNLL (175) |
| FLASFVEKTVL (32) | SPVVHQSL (80) | TLRSPGSSL (128) | GLRLIHVSL (176) |
| ILASHNLTV (33) | SPYRTPVL (81) | TVRGDVSSL (129) | GLRLLHVSL (177) |
| IQLTSVHLL (34) | SVFAGVVGV (82) | ALDPLLLRI (130) | LHNEKGLSL (178) |
| LELISFLPVL (35) | SYSPAHARL (83) | ISLIVTGLK (131) | LPSFSRPSGII (179) |
| NFCMLHQSI (36) | THGSEQLHL (84) | KILDVNLRI (132) | LSSRLPLGK (180) |
| PARPAGPL (37) | TQAPPNVVL (85) | ERVYIRASL (133) | MIGIKRLL (181) |
| PLPIVPAL (38) | VLVPYEPPQV (86) | ILDLESRY (134) | NLKKREIL (182) |
| SNLIRTGSH (39) | VSFPDVRKV (87) | KTFVQQKTL (135) | RMVAYLQQL (183) |
| VPAPAQAI (40) | VVFDKSDLAKY (88) | LYIKSLPAL (136) | SPARALPSL (184) |
| KGHGGPRSW (41) | YSHHSGLEY (89) | VLKEKNASL (137) | TVPGIQRY (185) |
| ITSSAVTTALK (42) | YYLDWIHHY (90) | LGISLTLKY (138) | VSRNYVLLI (186) |
| LLLPESPSI (43) | SVYKYLKAK (91) | DLLPKKLL (139) | LTVPLSVFW (187) |
| VILIPLPPK (44) | IYQFIMDRF (92) | HSLISIVYL (140) | KLNQAFLVL (188) |
| AVLLPKPPK (45) | GTLQGIRAW (93) | IAGALRSVL (141) | RLVSSTLLQK (189) |
| TQVSMAESI (46) | AQKVSVGQAA (94) | IGNPILRVL (142) | LPSHSLLI (190) |
| LNHLRTSI (47) | LYPSKLTHF (95) | IYAPHIRLS (143) | |
| NTSHLPLIY (48) | ATQNTIIGK (96) | LRSQILSY (144) | |

In general, peptides such as TAPs presented in the context of HLA class I vary in length from about 7 or 8 to about 15, or preferably 8 to 14 amino acid residues. In some embodiments of the methods of the disclosure, longer peptides comprising the TAP sequences defined herein are artificially loaded into cells such as antigen presenting cells (APCs), processed by the cells and the TAP is presented by MHC class I molecules at the surface of the APC. In this method, peptides/polypeptides longer than 15 amino acid residues can be loaded into APCs, are processed by proteases in the APC cytosol providing the corresponding TAP as defined herein for presentation. In some embodiments, the precursor peptide/polypeptide that is used to generate the TAP defined herein is for example 1000, 500, 400, 300, 200, 150, 100, 75, 50, 45, 40, 35, 30, 25, 20 or 15 amino acids or less. Thus, all the methods and processes using the TAPs described herein include the use of longer peptides or polypeptides (including the native protein), i.e. tumor antigen precursor peptides/polypeptides, to induce the presentation of the “final” 8-14 TAP following processing by the cell (APCs). In some embodiments, the herein-mentioned TAP is about 8 to 14, 8 to 13, or 8 to 12 amino acids long (e.g., 8, 9, 10, 11, 12 or 13 amino acids long), small enough for a direct fit in an HLA class I molecule. In an embodiment, the TAP comprises 20 amino acids or less, preferably 15 amino acids or less, more preferably 14 amino acids or less. In an embodiment, the TAP comprises at least 7 amino acids, preferably at least 8 amino acids or less, more preferably at least 9 amino acids.

The term “amino acid” as used herein includes both L- and D-isomers of the naturally occurring amino acids as well as other amino acids (e.g., naturally-occurring amino acids, non-naturally-occurring amino acids, amino acids which are not encoded by nucleic acid sequences, etc.) used in peptide chemistry to prepare synthetic analogs of TAPs. Examples of naturally occurring amino acids are glycine, alanine, valine, leucine, isoleucine, serine, threonine, etc. Other amino acids include for example non-genetically encoded forms of amino acids, as well as a conservative substitution of an L-amino acid. Naturally-occurring non-genetically encoded amino acids include, for example, beta-alanine, 3-amino-propionic acid, 2,3-diaminopropionic acid, alpha-aminoisobutyric acid (Aib), 4-amino-butyric acid, N-methylglycine (sarcosine), hydroxyproline, ornithine (e.g., L-ornithine), citrulline, t-butylalanine, t-butylglycine, N-methylisoleucine, phenylglycine, cyclohexylalanine, norleucine (Nle), norvaline, 2-napthylalanine, pyridylalanine, 3-benzothienyl alanine, 4-chlorophenylalanine, 2-fluorophenylalanine, 3-fluorophenylalanine, 4-fluorophenylalanine, penicillamine, 1,2,3,4-tetrahydro-isoquinoline-3-carboxylix acid, beta-2-thienylalanine, methionine sulfoxide, L-homoarginine (Hoarg), N-acetyl lysine, 2-amino butyric acid, 2-amino butyric acid, 2,4,-diaminobutyric acid (D- or L-), p-aminophenylalanine, N-methylvaline, homocysteine, homoserine (HoSer), cysteic acid, epsilon-amino hexanoic acid, delta-amino valeric acid, or 2,3-diaminobutyric acid (D- or L-), etc. These amino acids are well known in the art of biochemistry/peptide chemistry. In an embodiment, the TAP comprises only naturally-occurring amino acids.

In embodiments, the TAPs described herein include peptides with altered sequences containing substitutions of functionally equivalent amino acid residues, relative to the herein-mentioned sequences. For example, one or more amino acid residues within the sequence can be substituted by another amino acid of a similar polarity (having similar physico-chemical properties) which acts as a functional equivalent, resulting in a silent alteration. Substitution for an amino acid within the sequence may be selected from other members of the class to which the amino acid belongs. For example, positively charged (basic) amino acids include arginine, lysine and histidine (as well as homoarginine and ornithine). Nonpolar (hydrophobic) amino acids include leucine, isoleucine, alanine, phenylalanine, valine, proline, tryptophan and methionine. Uncharged polar amino acids include serine, threonine, cysteine, tyrosine, asparagine and glutamine. Negatively charged (acidic) amino acids include glutamic acid and aspartic acid. The amino acid glycine may be included in either the nonpolar amino acid family or the uncharged (neutral) polar amino acid family. Substitutions made within a family of amino acids are generally understood to be conservative substitutions. The herein-mentioned TAP may comprise all L-amino acids, all D-amino acids or a mixture of L- and D-amino acids. In an embodiment, the herein-mentioned TAP comprises all L-amino acids.

In an embodiment, in the sequences of the TAPs comprising or consisting of one of sequences of SEQ ID NOs: 1-190, preferably SEQ ID NOs: 97-154, the amino acid residues that do not substantially contribute to interactions with the T-cell receptor may be modified by replacement with other amino acid whose incorporation does not substantially affect T-cell reactivity and does not eliminate binding to the relevant MHC.

The TAP may also be N- and/or C-terminally capped or modified to prevent degradation, increase stability, affinity and/or uptake. Thus, in another aspect, the present disclosure provides a modified TAP of the formula Z¹-X-Z², wherein X is a TAP comprising, or consisting of, one of the amino acid sequences of SEQ ID NOs: 1-190, preferably SEQ ID NOs: 97-154.

In an embodiment, the amino terminal residue (i.e., the free amino group at the N-terminal end) of the TAP is modified (e.g., for protection against degradation), for example by covalent attachment of a moiety/chemical group (Z¹). Z¹ may be a straight chained or branched alkyl group of one to eight carbons, or an acyl group (R—CO—), wherein R is a hydrophobic moiety (e.g., acetyl, propionyl, butanyl, iso-propionyl, or iso-butanyl), or an aroyl group (Ar—CO—), wherein Ar is an aryl group. In an embodiment, the acyl group is a C₁-C₁₆ or C₃-C₁₆ acyl group (linear or branched, saturated or unsaturated), in a further embodiment, a saturated C₁-C₆ acyl group (linear or branched) or an unsaturated C₃-C₆ acyl group (linear or branched), for example an acetyl group (CH₃—CO—, Ac). In an embodiment, Z¹ is absent. The carboxy terminal residue (i.e., the free carboxy group at the C-terminal end of the TAP) of the TAP may be modified (e.g., for protection against degradation), for example by amidation (replacement of the OH group by a NH₂ group), thus in such a case Z² is a NH₂ group. In an embodiment, Z² may be an hydroxamate group, a nitrile group, an amide (primary, secondary or tertiary) group, an aliphatic amine of one to ten carbons such as methyl amine, iso-butylamine, iso-valerylamine or cyclohexylamine, an aromatic or arylalkyl amine such as aniline, napthylamine, benzylamine, cinnamylamine, or phenylethylamine, an alcohol or CH₂OH. In an embodiment, Z² is absent. In an embodiment, the TAP comprises one of the amino acid sequences of SEQ ID NOs: 1-190, preferably SEQ ID NOs: 97-154. In an embodiment, the TAP consists of one of the amino acid sequences of SEQ ID NOs: 1-190, preferably SEQ ID NOs: 97-154, i.e. wherein Z¹ and Z² are absent.

In another aspect, the present disclosure provides a leukemia TAP (or tumor-specific peptide), preferably an AML TAP, binding to an HLA-A*01:01 molecule, comprising or consisting of the sequence of SEQ ID NO:48, 67, 89, 134, 151 or 164, SEQ ID NO:134 or 151.

In another aspect, the present disclosure provides a leukemia TAP (or tumor-specific peptide), preferably an AML TAP, binding to an HLA-A*02:01 molecule, comprising or consisting of the sequence of SEQ ID NO:7, 11, 27, 32, 33, 34, 35, 4351, 52, 53, 54, 61, 65, 72, 77, 82, 86, 104, 108, 119, 123, 130, 132, 146, 150, 167, 168, 169, 171, 183, or 188, preferably SEQ ID NO: 104, 108, 119, 123, 130, 132, 146 or 150. Because HLA alleles show promiscuity (certain HLA alleles present similar epitopes, see Table 4), the above-identified TAP may further bind to HLA-A*02:05, HLA-A*02:06 and/or HLA-A*02:07 molecules.

In another aspect, the present disclosure provides a leukemia TAP (or tumor-specific peptide), preferably an AML TAP, binding to an HLA-A*03:01 molecule, comprising or consisting of the sequence of SEQ ID NO:5, 18, 19, 20, 42, 44, 74, 91, 105, 113, 126, 131, 159, 160, 180 or 189, preferably SEQ ID NO: 105, 113, 126 or 131. Because HLA alleles show promiscuity (certain HLA alleles present similar epitopes, see Table 4), the above-identified TAP may further bind to HLA-A*11:01 molecules.

In another aspect, the present disclosure provides a leukemia TAP (or tumor-specific peptide), preferably an AML TAP, binding to an HLA-A*11:01 molecule, comprising or consisting of the sequence of SEQ ID NO:6, 45, 96, 106, 121, 149 or 152, preferably SEQ ID NO: 106, 121, 149 or 152. Because HLA alleles show promiscuity (certain HLA alleles present similar epitopes, see Table 4), the above-identified TAP may further bind to HLA-A*03:01, HLA-A*31:01 and/or HLA-A*68:01 molecules.

In another aspect, the present disclosure provides a leukemia TAP (or tumor-specific peptide), preferably an AML TAP, binding to an HLA-A*24:02 molecule, comprising or consisting of the sequence of SEQ ID NO:13, 36, 71, 92, 95 or 145, preferably SEQ ID NO:145. Because HLA alleles show promiscuity (certain HLA alleles present similar epitopes, see Table 4), the above-identified TAP may further bind to HLA-A*23:01 molecules.

In another aspect, the present disclosure provides a leukemia TAP (or tumor-specific peptide), preferably an AML TAP, binding to an HLA-A*26:01 molecule, comprising or consisting of the sequence of SEQ ID NO:59 or SEQ ID NO: 185. Because HLA alleles show promiscuity (certain HLA alleles present similar epitopes, see Table 4), the above-identified TAP may further bind to HLA-A*25:01 and/or HLA-A*66:01 molecules.

In another aspect, the present disclosure provides a leukemia TAP (or tumor-specific peptide), preferably an AML TAP, binding to an HLA-A*29:02 molecule, comprising or consisting of the sequence of SEQ ID NO:88, 99 or 138, preferably SEQ ID NO:99 or 138. Because HLA alleles show promiscuity (certain HLA alleles present similar epitopes, see Table 4), the above-identified TAP may further bind to HLA-A*30:02 and/or HLA-B*15:02 molecules.

In another aspect, the present disclosure provides a leukemia TAP (or tumor-specific peptide), preferably an AML TAP, binding to an HLA-A*30:01 molecule, comprising or consisting of the sequence of SEQ ID NO:26, 29 or 165.

In another aspect, the present disclosure provides a leukemia TAP (or tumor-specific peptide), preferably an AML TAP, binding to an HLA-A*68:02 molecule, comprising or consisting of the sequence of SEQ ID NO:50 or SEQ ID NO: 148, preferably SEQ ID NO: 148.

In another aspect, the present disclosure provides a leukemia TAP (or tumor-specific peptide), preferably an AML TAP, binding to an HLA-B*07:02 molecule, comprising or consisting of the sequence of SEQ ID NO:8, 25, 40, 55, 56, 73, 78, 79, 80, 81, 98, 100, 107, 110, 111, 118, 120, 128, 129, 157, 161, 163, 179 or 184, preferably SEQ ID NO:98, 100, 107, 110, 111, 118, 120, 128 or 129. Because HLA alleles show promiscuity (certain HLA alleles present similar epitopes, see Table 4), the above-identified TAP may further bind to HLA-B*35:02, HLA-B*35:03, HLA-B*55:01 and/or HLA-B*56:01 molecules.

In another aspect, the present disclosure provides a leukemia TAP (or tumor-specific peptide), preferably an AML TAP, binding to an HLA-B*08:01 molecule, comprising or consisting of the sequence of SEQ ID NO:4, 14, 15, 17, SEQ ID NO:21, 22, 23, 28, 31, 47, 49, 101, 103, 114, 137, 139, 147, 156, 170, 181 or 182, preferably SEQ ID NO:101, 103, 114, 137, 139 or 147.

In another aspect, the present disclosure provides a leukemia TAP (or tumor-specific peptide), preferably an AML TAP, binding to an HLA-B*14:01 molecule, comprising or consisting of the sequence of SEQ ID NO:2, 39, 58, 66, 124, 133, 136, 141, 162, 175, 176 or 177, preferably SEQ ID NO:124, 133, 136 or 141.

In another aspect, the present disclosure provides a leukemia TAP (or tumor-specific peptide), preferably an AML TAP, binding to an HLA-B*15:01 molecule, comprising or consisting of the sequence of SEQ ID NO:10, 57, 62 or 94. Because HLA alleles show promiscuity (certain HLA alleles present similar epitopes, see Table 4), the above-identified TAP may further bind to HLA-B*15:02, HLA-B*15:03 and/or HLA-B*46:01 molecules.

In another aspect, the present disclosure provides a leukemia TAP (or tumor-specific peptide), preferably an AML TAP, binding to an HLA-B*27:05 molecule, comprising or consisting of the sequence of SEQ ID NO:1 or 144, preferably SEQ ID NO:144. Because HLA alleles show promiscuity (certain HLA alleles present similar epitopes, see Table 4), the above-identified TAP may further bind to HLA-B*27:02 molecules.

In another aspect, the present disclosure provides a leukemia TAP (or tumor-specific peptide), preferably an AML TAP, binding to an HLA-B*38:01 molecule, comprising or consisting of the sequence of SEQ ID NO:4, 64 or 84. Because HLA alleles show promiscuity (certain HLA alleles present similar epitopes, see Table 4), the above-identified TAP may further bind to HLA-B*39:01 molecules.

In another aspect, the present disclosure provides a leukemia TAP (or tumor-specific peptide), preferably an AML TAP, binding to an HLA-B*40:01 molecule, comprising or consisting of the sequence of SEQ ID NO:75 or 76. Because HLA alleles show promiscuity (certain HLA alleles present similar epitopes, see Table 4), the above-identified TAP may further bind to HLA-B*18:01, HLA-B*40:02, HLA-B*41:02, HLA-B*44:02, HLA-B*44:03 and/or HLA-B*45:01 molecules.

In another aspect, the present disclosure provides a leukemia TAP (or tumor-specific peptide), preferably an AML TAP, binding to an HLA-B*44:03 molecule, comprising or consisting of the sequence of SEQ ID NO: 127. Because HLA alleles show promiscuity (certain HLA alleles present similar epitopes, see Table 4), the above-identified TAP may further bind to HLA-B*18:01, HLA-B*40:01, HLA-B*40:02, HLA-B*41:02, HLA-B*44:02 and/or HLA-B*45:01 molecules.

In another aspect, the present disclosure provides a leukemia TAP (or tumor-specific peptide), preferably an AML TAP, binding to an HLA-B*51:01 molecule, comprising or consisting of the sequence of SEQ ID NO:12, 24, 38, 68, 115, 116 or 190, preferably SEQ ID NO:115 or 116. Because HLA alleles show promiscuity (certain HLA alleles present similar epitopes, see Table 4), the above-identified TAP may further bind to HLA-B*35:02, HLA-B*35:03, HLA-B*52:01, HLA-B*53:01, HLA-B*55:01 and/or HLA-B*56:01 molecules.

In another aspect, the present disclosure provides a leukemia TAP (or tumor-specific peptide), preferably an AML TAP, binding to an HLA-B*57:01 molecule, comprising or consisting of the sequence of SEQ ID NO: 3, 16, 41, 63, 69, 93, 122, 125, 135, 153 or 183, preferably 122, 125, 135 or 153. Because HLA alleles show promiscuity (certain HLA alleles present similar epitopes, see Table 4), the above-identified TAP may further bind to HLA-A*32:01 and/or HLA-B*58:01 molecules.

In another aspect, the present disclosure provides a leukemia TAP (or tumor-specific peptide), preferably an AML TAP, binding to an HLA-B*57:03 molecule, comprising or consisting of the sequence of SEQ ID NO: 60 or 172.

In another aspect, the present disclosure provides a leukemia TAP (or tumor-specific peptide), preferably an AML TAP, binding to an HLA-C*03:03 molecule, comprising or consisting of the sequence of SEQ ID NO:37, 112 or 140, preferably SEQ ID NO:112 or 140. Because HLA alleles show promiscuity (certain HLA alleles present similar epitopes, see Table 4), the above-identified TAP may further bind to HLA-B*46:01, HLA-C*03:02, HLA-C*03:04, HLA-C*08:01, HLA-C*08:02, HLA-C*12:02, HLA-C*12:03, HLA-C*15:02 and/or HLA-C*16:01 molecules.

In another aspect, the present disclosure provides a leukemia TAP (or tumor-specific peptide), preferably an AML TAP, binding to an HLA-C*05:01 molecule, comprising or consisting of the sequence of SEQ ID NO: 150. Because HLA alleles show promiscuity (certain HLA alleles present similar epitopes, see Table 4), the above-identified TAP may further bind to HLA-C*08:01 and/or HLA-C*08:02 molecules.

In another aspect, the present disclosure provides a leukemia TAP (or tumor-specific peptide), preferably an AML TAP, binding to an HLA-C*06:02 molecule, comprising or consisting of the sequence of SEQ ID NO:9, 70, 87, 142, 154 or 166, preferably SEQ ID NO:142 or 154. Because HLA alleles show promiscuity (certain HLA alleles present similar epitopes, see Table 4), the above-identified TAP may further bind to HLA-B*27:02, HLA-C*07:01 and/or HLA-C*07:02 molecules.

In another aspect, the present disclosure provides a leukemia TAP (or tumor-specific peptide), preferably an AML TAP, binding to an HLA-C*07:01 molecule, comprising or consisting of the sequence of SEQ ID NO: 142, 143, 155, 178 or 186), preferably SEQ ID NO:142 or 143. Because HLA alleles show promiscuity (certain HLA alleles present similar epitopes, see Table 4), the above-identified TAP may further bind to HLA-B*27:02, HLA-C*07:01, HLA-C*07:02 and/or HLA-C*14:02 molecules.

In another aspect, the present disclosure provides a leukemia TAP (or tumor-specific peptide), preferably an AML TAP, binding to an HLA-C*07:02 molecule, comprising or consisting of the sequence of SEQ ID NO:30, 83, 85, 90, 109, 117 or 158, preferably SEQ ID NO:109 or 117. Because HLA alleles show promiscuity (certain HLA alleles present similar epitopes, see Table 4), the above-identified TAP may further bind to HLA-B*27:02, HLA-C*07:01, HLA-C*07:02 and/or HLA-C*14:02 molecules.

In another aspect, the present disclosure provides a leukemia TAP (or tumor-specific peptide), preferably an AML TAP, binding to an HLA-C*08:02 molecule, comprising or consisting of the sequence of SEQ ID NO:97, 102 or 174, preferably SEQ ID NO:97 or 102. Because HLA alleles show promiscuity (certain HLA alleles present similar epitopes, see Table 4), the above-identified TAP may further bind to HLA-C*03:03, HLA-C*03:04, HLA-C*05:01, HLA-C*08:01 and/or HLA-C*15:02 molecules.

In another aspect, the present disclosure provides a leukemia TAP (or tumor-specific peptide), preferably an AML TAP, binding to an HLA-C*12:03 molecule, comprising or consisting of the sequence of SEQ ID NO: 173. Because HLA alleles show promiscuity (certain HLA alleles present similar epitopes, see Table 4), the above-identified TAP may further bind to HLA-B*46:01, HLA-C*03:02, HLA-C*03:03, HLA-C*03:04, HLA-C*08:01, HLA-C*12:03, HLA-C*15:02 and/or HLA-C*16:01 molecules.

In an embodiment, the TAP is encoded by a sequence located in an untranslated transcribed region (UTR), i.e. a 3′-UTR or 5′-UTR region. In another embodiment, the TAP is encoded by a sequence located in an intron. In another embodiment, the TAP is encoded by a sequence located in an intergenic region. In another embodiment, the TAP is encoded by a sequence located in an exon and originates from a frameshift.

The TAPs of the disclosure may be produced by expression in a host cell comprising a nucleic acid encoding the TAPs (recombinant expression) or by chemical synthesis (e.g., solid-phase peptide synthesis). Peptides can be readily synthesized by manual and/or automated solid phase procedures well known in the art. Suitable syntheses can be performed for example by utilizing “T-boc” or “Fmoc” procedures. Techniques and procedures for solid phase synthesis are described in for example Solid Phase Peptide Synthesis: A Practical Approach, by E. Atherton and R. C. Sheppard, published by IRL, Oxford University Press, 1989. Alternatively, the MiHA peptides may be prepared by way of segment condensation, as described, for example, in Liu et al., Tetrahedron Lett. 37: 933-936, 1996; Baca et al., J. Am. Chem. Soc. 117: 1881-1887, 1995; Tam etal., Int. J. Peptide Protein Res. 45: 209-216, 1995; Schnolzerand Kent, Science 256: 221-225, 1992; Liu and Tam, J. Am. Chem. Soc. 116: 4149-4153, 1994; Liu and Tam, Proc. Natl. Acad. Sci. USA 91: 6584-6588, 1994; and Yamashiro and Li, Int. J. Peptide Protein Res. 31: 322-334, 1988). Other methods useful for synthesizing the TAPs are described in Nakagawa et al., J. Am. Chem. Soc. 107: 7087-7092, 1985. In an embodiment, the TAP is chemically synthesized (synthetic peptide). Another embodiment of the present disclosure relates to a non-naturally occurring peptide wherein said peptide consists or consists essentially of an amino acid sequences defined herein and has been synthetically produced (e.g. synthesized) as a pharmaceutically acceptable salt. The salts of the TAPs according to the present disclosure differ substantially from the peptides in their state(s) in vivo, as the peptides as generated in vivo are no salts. The non-natural salt form of the peptide may modulate the solubility of the peptide, in particular in the context of pharmaceutical compositions comprising the peptides, e.g. the peptide vaccines as disclosed herein. Preferably, the salts are pharmaceutically acceptable salts of the peptides.

In an embodiment, the herein-mentioned TAP is substantially pure. A compound is “substantially pure” when it is separated from the components that naturally accompany it. Typically, a compound is substantially pure when it is at least 60%, more generally 75%, 80% or 85%, preferably over 90% and more preferably over 95%, by weight, of the total material in a sample. Thus, for example, a polypeptide that is chemically synthesized or produced by recombinant technology will generally be substantially free from its naturally associated components, e.g. components of its source macromolecule. A nucleic acid molecule is substantially pure when it is not immediately contiguous with (i.e., covalently linked to) the coding sequences with which it is normally contiguous in the naturally occurring genome of the organism from which the nucleic acid is derived. A substantially pure compound can be obtained, for example, by extraction from a natural source; by expression of a recombinant nucleic acid molecule encoding a peptide compound; or by chemical synthesis. Purity can be measured using any appropriate method such as column chromatography, gel electrophoresis, HPLC, etc. In an embodiment, the TAP is in solution. In another embodiment, the TAP is in solid form, e.g., lyophilized.

In another aspect, the disclosure further provides a nucleic acid (isolated) encoding the herein-mentioned TAPs or a tumor antigen precursor-peptide. In an embodiment, the nucleic acid comprises from about 21 nucleotides to about 45 nucleotides, from about 24 to about 45 nucleotides, for example 24, 27, 30, 33, 36, 39, 42 or 45 nucleotides. “Isolated”, as used herein, refers to a peptide or nucleic molecule separated from other components that are present in the natural environment of the molecule or a naturally occurring source macromolecule (e.g., including other nucleic acids, proteins, lipids, sugars, etc.). “Synthetic”, as used herein, refers to a peptide or nucleic molecule that is not isolated from its natural sources, e.g., which is produced through recombinant technology or using chemical synthesis. A nucleic acid of the disclosure may be used for recombinant expression of the TAP of the disclosure, and may be included in a vector or plasmid, such as a cloning vector or an expression vector, which may be transfected into a host cell. In an embodiment, the disclosure provides a cloning, expression or viral vector or plasmid comprising a nucleic acid sequence encoding the TAP of the disclosure. Alternatively, a nucleic acid encoding a TAP of the disclosure may be incorporated into the genome of the host cell. In either case, the host cell expresses the TAP or protein encoded by the nucleic acid. The term “host cell” as used herein refers not only to the particular subject cell, but to the progeny or potential progeny of such a cell. A host cell can be any prokaryotic (e.g., E. coli) or eukaryotic cell (e.g., insect cells, yeast or mammalian cells) capable of expressing the TAPs described herein. The vector or plasmid contains the necessary elements for the transcription and translation of the inserted coding sequence, and may contain other components such as resistance genes, cloning sites, etc. Methods that are well known to those skilled in the art may be used to construct expression vectors containing sequences encoding peptides or polypeptides and appropriate transcriptional and translational control/regulatory elements operably linked thereto. These methods include in vitro recombinant DNA techniques, synthetic techniques, and in vivo genetic recombination. Such techniques are described in Sambrook. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y. “Operably linked” refers to a juxtaposition of components, particularly nucleotide sequences, such that the normal function of the components can be performed. Thus, a coding sequence that is operably linked to regulatory sequences refers to a configuration of nucleotide sequences wherein the coding sequences can be expressed under the regulatory control, that is, transcriptional and/or translational control, of the regulatory sequences. “Regulatory/control region” or “regulatory/control sequence”, as used herein, refers to the non-coding nucleotide sequences that are involved in the regulation of the expression of a coding nucleic acid. Thus, the term regulatory region includes promoter sequences, regulatory protein binding sites, upstream activator sequences, and the like. The vector (e.g., expression vector) may have the necessary 5′ upstream and 3′ downstream regulatory elements such as promoter sequences such as CMV, PGK and EFla promoters, ribosome recognition and binding TATA box, and 3′ UTR AAUAAA transcription termination sequence for the efficient gene transcription and translation in its respective host cell. Other suitable promoters include the constitutive promoter of simian vims 40 (SV40) early promoter, mouse mammary tumor virus (MMTV), HIV LTR promoter, MoMuLV promoter, avian leukemia virus promoter, EBV immediate early promoter, and Rous sarcoma vims promoter. Human gene promoters may also be used, including, but not limited to the actin promoter, the myosin promoter, the hemoglobin promoter, and the creatine kinase promoter. In certain embodiments inducible promoters are also contemplated as part of the vectors expressing the TAP. This provides a molecular switch capable of turning on expression of the polynucleotide sequence of interest or turning off expression. Examples of inducible promoters include, but are not limited to a metallothionine promoter, a glucocorticoid promoter, a progesterone promoter, or a tetracycline promoter. Examples of vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or PI-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are Lenti-X™ Bicistronic Expression System (Neo) vectors (Clontrch), pClneo vectors (Promega) for expression in mammalian cells; pLenti4/V5-DEST™, pLenti6/V5-DEST™, and pLenti6.2N5-GW/lacZ (Invitrogen) for lentivirus-mediated gene transfer and expression in mammalian cells. The coding sequences of the TAPs disclosed herein can be ligated into such expression vectors for the expression of the TAP in mammalian cells.

In certain embodiments, the nucleic acids encoding the TAP of the present disclosure are provided in a viral vector. A viral vector can be those derived from retrovirus, lentivirus, or foamy virus. As used herein, the term, “viral vector,” refers to a nucleic acid vector construct that includes at least one element of viral origin and has the capacity to be packaged into a viral vector particle. The viral vector can contain the coding sequence for the various proteins described herein in place of nonessential viral genes. The vector and/or particle can be utilized for the purpose of transferring DNA, RNA or other nucleic acids into cells either in vitro or in vivo. Numerous forms of viral vectors are known in the art.

In embodiment, the nucleic acid (DNA, RNA) encoding the TAP of the disclosure is comprised within a liposome or any other suitable vehicle.

In another aspect, the present disclosure provides an MHC class I molecule comprising (i.e. presenting or bound to) one or more of the TAP of SEQ ID NOs: 1-190, preferably SEQ ID NOs: 97-154. In an embodiment, the MHC class I molecule is an HLA-A1 molecule, in a further embodiment an HLA-A*01:01 molecule. In another embodiment, the MHC class I molecule is an HLA-A2 molecule, in a further embodiment an HLA-A*02:01 molecule. In another embodiment, the MHC class I molecule is an HLA-A3 molecule, in a further embodiment an HLA-A*03:01 molecule. In another embodiment, the MHC class I molecule is an HLA-A11 molecule, in a further embodiment an HLA-A*11:01 molecule. In another embodiment, the MHC class I molecule is an HLA-A24 molecule, in a further embodiment an HLA-A*24:02 molecule. In another embodiment, the MHC class I molecule is an HLA-A26 molecule, in a further embodiment an HLA-A*26:01 molecule. In another embodiment, the MHC class I molecule is an HLA-A29 molecule, in a further embodiment an HLA-A*29:02 molecule. In another embodiment, the MHC class I molecule is an HLA-A30 molecule, in a further embodiment an HLA-A*30:01 molecule. In another embodiment, the MHC class I molecule is an HLA-A68 molecule, in a further embodiment an HLA-A*68:02 molecule. In another embodiment, the MHC class I molecule is an HLA-B07 molecule, in a further embodiment an HLA-B*07:02 molecule. In another embodiment, the MHC class I molecule is an HLA-B08 molecule, in a further embodiment an HLA-B*08:01 molecule. In another embodiment, the MHC class I molecule is an HLA-B14 molecule, in a further embodiment an HLA-B*14:01 molecule. In another embodiment, the MHC class I molecule is an HLA-B15 molecule, in a further embodiment an HLA-B*15:01 molecule. In another embodiment, the MHC class I molecule is an HLA-B27 molecule, in a further embodiment an HLA-B*27:05 molecule. In another embodiment, the MHC class I molecule is an HLA-B38 molecule, in a further embodiment an HLA-B*38:01 molecule. In another embodiment, the MHC class I molecule is an HLA-B40 molecule, in a further embodiment an HLA-B*40:01 molecule. In another embodiment, the MHC class I molecule is an HLA-B44 molecule, in a further embodiment an HLA-B*44:02 or HLA-B*44:03 molecule. In another embodiment, the MHC class I molecule is an HLA-B57 molecule, in a further embodiment an HLA-B*57:01 or HLA-B*57:03 molecule. In another embodiment, the MHC class I molecule is an HLA-C03 molecule, in a further embodiment an HLA-C*03:03 molecule. In another embodiment, the MHC class I molecule is an HLA-C04 molecule, in a further embodiment an HLA-C*04:01 molecule. In another embodiment, the MHC class I molecule is an HLA-C05 molecule, in a further embodiment an HLA-C*05:01 molecule. In another embodiment, the MHC class I molecule is an HLA-C06 molecule, in a further embodiment an HLA-C*06:02 molecule. In another embodiment, the MHC class I molecule is an HLA-C07 molecule, in a further embodiment an HLA-C*07:01 or HLA-C*07:02 molecule. In another embodiment, the MHC class I molecule is an HLA-C08 molecule, in a further embodiment an HLA-C*08:02 molecule. In another embodiment, the MHC class I molecule is an HLA-C12 molecule, in a further embodiment an HLA-C*12:03 molecule.

In an embodiment, the TAP is non-covalently bound to the MHC class I molecule (i.e., the TAP is loaded into, or non-covalently bound to the peptide binding groove/pocket of the MHC class I molecule). In another embodiment, the TAP is covalently attached/bound to the MHC class I molecule (alpha chain). In such a construct, the TAP and the MHC class I molecule (alpha chain) are produced as a synthetic fusion protein, typically with a short (e.g., 5 to 20 residues, preferably about 8-12, e.g., 10) flexible linker or spacer (e.g., a polyglycine linker). In another aspect, the disclosure provides a nucleic acid encoding a fusion protein comprising a TAP defined herein fused to a MHC class I molecule (alpha chain). In an embodiment, the MHC class I molecule (alpha chain) - peptide complex is multimerized. Accordingly, in another aspect, the present disclosure provides a multimer of MHC class I molecule loaded (covalently or not) with the herein-mentioned TAP. Such multimers may be attached to a tag, for example a fluorescent tag, which allows the detection of the multimers. A great number of strategies have been developed for the production of MHC multimers, including MHC dimers, tetramers, pentamers, octamers, etc. (reviewed in Bakker and Schumacher, Current Opinion in Immunology 2005, 17:428-433). MHC multimers are useful, for example, for the detection and purification of antigen-specific T cells. Thus, in another aspect, the present disclosure provides a method for detecting or purifying (isolating, enriching) CD8⁺ T lymphocytes specific for a TAP defined herein, the method comprising contacting a cell population with a multimer of MHC class I molecule loaded (covalently or not) with the TAP; and detecting or isolating the CD8⁺ T lymphocytes bound by the MHC class I multimers. CD8⁺ T lymphocytes bound by the MHC class I multimers may be isolated using known methods, for example fluorescence activated cell sorting (FACS) or magnetic activated cell sorting (MACS).

In yet another aspect, the present disclosure provides a cell (e.g., a host cell), in an embodiment an isolated cell, comprising the herein-mentioned nucleic acid, vector or plasmid of the disclosure, i.e. a nucleic acid or vector encoding one or more TAPs. In another aspect, the present disclosure provides a cell expressing at its surface an MHC class I molecule (e.g., an MHC class I molecule of one of the alleles disclosed above) bound to or presenting a TAP according to the disclosure. In one embodiment, the host cell is a eukaryotic cell, such as a mammalian cell, preferably a human cell. a cell line or an immortalized cell. In another embodiment, the cell is an antigen-presenting cell (APC). In one embodiment, the host cell is a primary cell, a cell line or an immortalized cell. In another embodiment, the cell is an antigen-presenting cell (APC). Nucleic acids and vectors can be introduced into cells via conventional transformation or transfection techniques. The terms “transformation” and “transfection” refer to techniques for introducing foreign nucleic acid into a host cell, including calcium phosphate or calcium chloride co-precipitation, DEAE-dextran-mediated transfection, lipofection, electroporation, microinjection and viral-mediated transfection. Suitable methods for transforming or transfecting host cells can for example be found in Sambrook et al. (supra), and other laboratory manuals. Methods for introducing nucleic acids into mammalian cells in vivo are also known, and may be used to deliver the vector or plasmid of the disclosure to a subject for gene therapy.

Cells such as APCs can be loaded with one or more TAPs using a variety of methods known in the art. As used herein “loading a cell” with a TAP means that RNA or DNA encoding the TAP, or the TAP, is transfected into the cells or alternatively that the APC is transformed with a nucleic acid encoding the TAP. The cell can also be loaded by contacting the cell with exogenous TAPs that can bind directly to MHC class I molecule present at the cell surface (e.g., peptide-pulsed cells). The TAPs may also be fused to a domain or motif that facilitates its presentation by MHC class I molecules, for example to an endoplasmic reticulum (ER) retrieval signal, a C-terminal Lys-Asp-Glu-Leu sequence (see Wang et al., Eur J Immunol. 2004 Dec;34(12):3582-94).

In another aspect, the present disclosure provides a composition or peptide combination/pool comprising any one of, or any combination of, the TAPs defined herein (or a nucleic acid encoding said peptide(s)). In an embodiment, the composition comprises any combination of the TAPs defined herein (any combination of 2, 3, 4, 5, 6, 7, 8, 9, 10 or more TAPs), or a combination of nucleic acids encoding said TAPs). Compositions comprising any combination/sub-combination of the TAPs defined herein are encompassed by the present disclosure. In another embodiment, the combination or pool may comprise one or more known tumor antigens.

Thus, in another aspect, the present disclosure provides a composition comprising any one of, or any combination of, the TAPs defined herein and a cell expressing a MHC class I molecule (e.g., a MHC class I molecule of one of the alleles disclosed above). APC for use in the present disclosure are not limited to a particular type of cell and include professional APCs such as dendritic cells (DCs), Langerhans cells, macrophages and B cells, which are known to present proteinaceous antigens on their cell surface so as to be recognized by CD8⁺ T lymphocytes. For example, an APC can be obtained by inducing DCs from peripheral blood monocytes and then contacting (stimulating) the TAPs, either in vitro, ex vivo or in vivo. APC can also be activated to present a TAP in vivo where one or more of the TAPs of the disclosure are administered to a subject and APCs that present a TAP are induced in the body of the subject. The phrase “inducing an APC” or “stimulating an APC” includes contacting or loading a cell with one or more TAPs, or nucleic acids encoding the TAPs such that the TAPs are presented at its surface by MHC class I molecules. As noted herein, according to the present disclosure, the TAPs may be loaded indirectly for example using longer peptides/polypeptides comprising the sequence of the TAPs (including the native protein), which is then processed (e.g., by proteases) inside the APCs to generate the TAP/MHC class I complexes at the surface of the cells. After loading APCs with TAPs and allowing the APCs to present the TAPs, the APCs can be administered to a subject as a vaccine. For example, the ex vivo administration can include the steps of: (a) collecting APCs from a first subject, (b) contacting/loading the APCs of step (a) with a TAP to form MHC class I/TAP complexes at the surface of the APCs; and (c) administering the peptide-loaded APCs to a second subject in need for treatment.

The first subject and the second subject may be the same subject (e.g., autologous vaccine), or may be different subjects (e.g., allogeneic vaccine). Alternatively, according to the present disclosure, use of a TAP described herein (or a combination thereof) for manufacturing a composition (e.g., a pharmaceutical composition) for inducing antigen-presenting cells is provided. In addition, the present disclosure provides a method or process for manufacturing a pharmaceutical composition for inducing antigen-presenting cells, wherein the method or the process includes the step of admixing or formulating the TAP, or a combination thereof, with a pharmaceutically acceptable carrier. Cells such as APCs expressing a MHC class I molecule (e.g., HLA-A1, HLA-A2, HLA-A3, HLA-A11, HLA-A24, HLA-A25, HLA-A29, HLA-A32, HLA-B07, HLA-B08, HLA-B14, HLA-B15, HLA-B18, HLA-B39, HLA-B40, HLA-B44, HLA-C03, HLA-C04, HLA-C05, HLA-C06, HLA-C07, HLA-C12, or HLA-C14 molecule) loaded with any one of, or any combination of, the TAPs defined herein, may be used for stimulating/amplifying CD8⁺ T lymphocytes, for example autologous CD8⁺ T lymphocytes. Accordingly, in another aspect, the present disclosure provides a composition comprising any one of, or any combination of, the TAPs defined herein (or a nucleic acid or vector encoding same); a cell expressing an MHC class I molecule and a T lymphocyte, more specifically a CD8⁺ T lymphocyte (e.g., a population of cells comprising CD8⁺ T lymphocytes).

In an embodiment, the composition further comprises a buffer, an excipient, a carrier, a diluent and/or a medium (e.g., a culture medium). In a further embodiment, the buffer, excipient, carrier, diluent and/or medium is/are pharmaceutically acceptable buffer(s), excipient(s), carrier(s), diluent(s) and/or medium (media). As used herein “pharmaceutically acceptable buffer, excipient, carrier, diluent and/or medium” includes any and all solvents, buffers, binders, lubricants, fillers, thickening agents, disintegrants, plasticizers, coatings, barrier layer formulations, lubricants, stabilizing agent, release-delaying agents, dispersion media, coatings, antibacterial and antifungal agents, isotonic agents, and the like that are physiologically compatible, do not interfere with effectiveness of the biological activity of the active ingredient(s) and that are not toxic to the subject. The use of such media and agents for pharmaceutically active substances is well known in the art (Rowe et al., Handbook of pharmaceutical excipients, 2003, 4^{th} edition, Pharmaceutical Press, London UK). Except insofar as any conventional media or agent is incompatible with the active compound (peptides, cells), use thereof in the compositions of the disclosure is contemplated. In an embodiment, the buffer, excipient, carrier and/or medium is a non-naturally occurring buffer, excipient, carrier and/or medium. In an embodiment, one or more of the TAPs defined herein, or the nucleic acids (e.g., mRNAs) encoding said one or more TAPs, are comprised within or complexed to a liposome, e.g., a cationic liposome (see, e.g., Vitor MT et al., Recent Pat Drug Deliv Formul. 2013 Aug;7(2):99-110) or suitable other carriers.

In another aspect, the present disclosure provides a composition comprising one of more of the any one of, or any combination of, the TAPs defined herein (or a nucleic acid encoding said peptide(s)), and a buffer, an excipient, a carrier, a diluent and/or a medium. For compositions comprising cells (e.g., APCs, T lymphocytes), the composition comprises a suitable medium that allows the maintenance of viable cells. Representative examples of such media include saline solution, Earl’s Balanced Salt Solution (Life Technologies®) or PlasmaLyte® ( Baxter International®). In an embodiment, the composition (e.g., pharmaceutical composition) is an “immunogenic composition”, “vaccine composition” or “vaccine”. The term “Immunogenic composition”, “vaccine composition” or “vaccine” as used herein refers to a composition or formulation comprising one or more TAPs or vaccine vector and which is capable of inducing an immune response against the one or more TAPs present therein when administered to a subject. Vaccination methods for inducing an immune response in a mammal comprise use of a vaccine or vaccine vector to be administered by any conventional route known in the vaccine field, e.g., via a mucosal (e.g., ocular, intranasal, pulmonary, oral, gastric, intestinal, rectal, vaginal, or urinary tract) surface, via a parenteral (e.g., subcutaneous, intradermal, intramuscular, intravenous, or intraperitoneal) route, or topical administration (e.g., via a transdermal delivery system such as a patch). In an embodiment, the TAP (or a combination thereof) is conjugated to a carrier protein (conjugate vaccine) to increase the immunogenicity of the TAP(s). The present disclosure thus provides a composition (conjugate) comprising a TAP (or a combination thereof), or a nucleic acid encoding the TAP or combination thereof, and a carrier protein. For example, the TAP(s) or nucleic acid(s) may be conjugated or complexed to a Toll-like receptor (TLR) ligand (see, e.g., Zom et al., Adv Immunol. 2012, 114: 177-201) or polymers/dendrimers (see, e.g., Liu et al., Biomacromolecules. 2013 Aug 12;14(8):2798-806). In an embodiment, the immunogenic composition or vaccine further comprises an adjuvant. “Adjuvant” refers to a substance which, when added to an immunogenic agent such as an antigen (TAPs, nucleic acids and/or cells according to the present disclosure), nonspecifically enhances or potentiates an immune response to the agent in the host upon exposure to the mixture. Examples of adjuvants currently used in the field of vaccines include (1) mineral salts (aluminum salts such as aluminum phosphate and aluminum hydroxide, calcium phosphate gels), squalene, (2) oil-based adjuvants such as oil emulsions and surfactant based formulations, e.g., MF59 (microfluidised detergent stabilised oil-in-water emulsion), QS21 (purified saponin), AS02 [SBAS2] (oil-in-water emulsion + MPL + QS-21), (3) particulate adjuvants, e.g., virosomes (unilamellar liposomal vehicles incorporating influenza haemagglutinin), AS04 ([SBAS4] aluminum salt with MPL), ISCOMS (structured complex of saponins and lipids), polylactide co-glycolide (PLG), (4) microbial derivatives (natural and synthetic), e.g., monophosphoryl lipid A (MPL), Detox (MPL + M. Phlei cell wall skeleton), AGP [RC-529] (synthetic acylated monosaccharide), DC_Chol (lipoidal immunostimulators able to self-organize into liposomes), OM-174 (lipid A derivative), CpG motifs (synthetic oligonucleotides containing immunostimulatory CpG motifs), modified LT and CT (genetically modified bacterial toxins to provide non-toxic adjuvant effects), (5) endogenous human immunomodulators, e.g., hGM-CSF or hIL-12 (cytokines that can be administered either as protein or plasmid encoded), Immudaptin (C3d tandem array) and/or (6) inert vehicles, such as gold particles, and the like.

In an embodiment, the TAP(s) or composition comprising same is/are in lyophilized form. In another embodiment, the TAP(s) or composition comprising same is/are in a liquid composition. In a further embodiment, the TAP(s) is/are at a concentration of about 0.01 µg/mL to about 100 µg/mL in the composition. In further embodiments, the TAP(s) is/are at a concentration of about 0.2 µg/mL to about 50 µg/mL, about 0.5 µg/mL to about 10, 20, 30, 40 or 50 µg/mL, about 1 µg/mL to about 10 µg/mL, or about 2 µg/mL, in the composition.

As noted herein, cells such as APCs that express an MHC class I molecule loaded with or bound to any one of, or any combination of, the TAPs defined herein, may be used for stimulating/amplifying CD8⁺ T lymphocytes in vivo or ex vivo. Accordingly, in another aspect, the present disclosure provides T cell receptor (TCR) molecules capable of interacting with or binding the herein-mentioned MHC class I molecule/ TAP complex, and nucleic acid molecules encoding such TCR molecules, and vectors comprising such nucleic acid molecules. A TCR according to the present disclosure is capable of specifically interacting with or binding a TAP loaded on, or presented by, an MHC class I molecule, preferably at the surface of a living cell in vitro or in vivo.

In an embodiment, the anti-leukemia (e.g., anti-AML) TCR according to the present disclosure comprises a TCRbeta (β) chain comprising a complementary determining region 3 (CDR3) comprising one of the amino acid sequences set forth in SEQ ID NOs: 191-219.

In an embodiment, the TCR is specific for one or more of the following TAPs: SLLSGLLRA, ALPVALPSL, ALDPLLLRI IASPIALL and/or SLDLLPLSI, and comprises a TCRβ chain comprising a CDR3 comprising one of the amino acid sequences set forth in SEQ ID NOs: 191-199. In an embodiment, the TCR is specific for the TAP SLLSGLLRA and comprises a TCRβ chain comprising a CDR3 comprising one of the amino acid sequences set forth in SEQ ID NOs: 191-199. In an embodiment, the TCR is specific for the TAP ALPVALPSL and comprises a TCRβ chain comprising a CDR3 comprising one of the amino acid sequences set forth in SEQ ID NOs: 191-199. In an embodiment, the TCR is specific for the TAP ALDPLLLRI and comprises a TCRβ chain comprising a CDR3 comprising one of the amino acid sequences set forth in SEQ ID NOs: 191-199. In an embodiment, the TCR is specific for the TAP IASPIALL and comprises a TCRβ chain comprising a CDR3 comprising one of the amino acid sequences set forth in SEQ ID NOs: 191-199. In an embodiment, the TCR is specific for the TAP SLDLLPLSI and comprises a TCRβ chain comprising a CDR3 comprising one of the amino acid sequences set forth in SEQ ID NOs: 191-199.

In another embodiment, the TCR is specific for one or more of the following TAPs: LTDRIYLTL, VLFGGKVSGA, LGISLTLKY, FNVALNARY and/or TLNQGINVYI, and comprises a TCRβ chain comprising a CDR3 comprising one of the amino acid sequences set forth in SEQ ID NOs: 200-209. In an embodiment, the TCR is specific for the TAP LTDRIYLTL and comprises a TCRβ chain comprising a CDR3 comprising one of the amino acid sequences set forth in SEQ ID NOs: 200-209. In an embodiment, the TCR is specific for the TAP VLFGGKVSGA and comprises a TCRβ chain comprising a CDR3 comprising one of the amino acid sequences set forth in SEQ ID NOs: 200-209. In an embodiment, the TCR is specific for the TAP LGISLTLKY and comprises a TCRβ chain comprising a CDR3 comprising one of the amino acid sequences set forth in SEQ ID NOs: 200-209. In an embodiment, the TCR is specific forthe TAP FNVALNARY and comprises a TCRβ chain comprising a CDR3 comprising one of the amino acid sequences set forth in SEQ ID NOs: 200-209. In an embodiment, the TCR is specific forthe TAP TLNQGINVYI and comprises a TCRβ chain comprising a CDR3 comprising one of the amino acid sequences set forth in SEQ ID NOs: 200-209.

In another embodiment, the TCR is specific for one or more of the following TAPs: LRSQILSY, KILDVNLRI, HSLISIVYL, KLQDKEIGL and/or AQDIILQAV, and comprises a TCRβ chain comprising a CDR3 comprising one of the amino acid sequences set forth in SEQ ID NOs: 210-219. In an embodiment, the TCR is specific for the TAP LRSQILSY and comprises a TCRβ chain comprising a CDR3 comprising one of the amino acid sequences set forth in SEQ ID NOs: 210-219. In an embodiment, the TCR is specific for the TAP KILDVNLRI and comprises a TCRβ chain comprising a CDR3 comprising one of the amino acid sequences set forth in SEQ ID NOs: 210-219. In an embodiment, the TCR is specific for the TAP HSLISIVYL and comprises a TCRβ chain comprising a CDR3 comprising one of the amino acid sequences set forth in SEQ ID NOs: 210-219. In an embodiment, the TCR is specific for the TAP KLQDKEIGL and comprises a TCRβ chain comprising a CDR3 comprising one of the amino acid sequences set forth in SEQ ID NOs: 210-219. In an embodiment, the TCR is specific for the TAP AQDIILQAV and comprises a TCRβ chain comprising a CDR3 comprising one of the amino acid sequences set forth in SEQ ID NOs: 210-219.

In an embodiment, the TCR according to the present disclosure recognizes one or more of the above-noted TAPs bound to HLA-A*02:01, HLA-A*29:02, HLA-B*15:01, HLA-B27:05, HLA-C*01:02, and/or HLA-C*03:04 molecules. In an embodiment, the TCR according to the present disclosure recognizes one or more of the above-noted TAPs bound to HLA-A*02:01 molecules. In an embodiment, the TCR according to the present disclosure recognizes one or more of the above-noted TAPs bound to HLA-A*29:02 molecules. In an embodiment, the TCR according to the present disclosure recognizes one or more of the above-noted TAPs bound to HLA -B*15:01 molecules. In an embodiment, the TCR according to the present disclosure recognizes one or more of the above-noted TAPs bound to HLA-B27:05 molecules. In an embodiment, the TCR according to the present disclosure recognizes one or more of the above-noted TAPs bound to HLA-C*01:02 molecules. In an embodiment, the TCR according to the present disclosure recognizes one or more of the above-noted TAPs bound to HLA-C*03:04 molecules.

The term TCR as used herein refers to an immunoglobulin superfamily member having a variable binding domain, a constant domain, a transmembrane region, and a short cytoplasmic tail; see, e.g., Janeway et al, Immunobiology: The Immune System in Health and Disease, 3rd Ed., Current Biology Publications, p. 4:33, 1997) capable of specifically binding to an antigen peptide bound to a MHC receptor. A TCR can be found on the surface of a cell and generally is comprised of a heterodimer having α and β chains (also known as TCRα and TCRβ, respectively). Like immunoglobulins, the extracellular portion of TCR chains (e.g., α-chain, β-chain) contain two immunoglobulin regions, a variable region (e.g., TCR variable α region or Vα and TCR variable β region or Vβ; typically amino acids 1 to 116 based on Rabat numbering at the N-terminus), and one constant region (e.g., TCR constant domain α or Cα and typically amino acids 117 to 259 based on Rabat, TCR constant domain β or Cβ, typically amino acids 117 to 295 based on Rabat) adjacent to the cell membrane. Also, like immunoglobulins, the variable domains contain complementary determining regions (CDRs. 3 in each chain) separated by framework regions (FRs). In certain embodiments, a TCR is found on the surface of T cells (or T lymphocytes) and associates with the CD3 complex.

A TCR and in particular nucleic acids encoding a TCR of the disclosure may for instance be applied to genetically transform/modify T lymphocytes (e.g., CD8⁺ T lymphocytes) or other types of lymphocytes generating new T lymphocyte clones that specifically recognize an MHC class I/TAP complex. In a particular embodiment, T lymphocytes (e.g., CD8⁺ T lymphocytes) obtained from a patient are transformed to express one or more TCRs that recognize a TAP and the transformed cells are administered to the patient (autologous cell transfusion). In a particular embodiment, T lymphocytes (e.g., CD8⁺ T lymphocytes) obtained from a donor are transformed to express one or more TCRs that recognize a TAP and the transformed cells are administered to a recipient (allogenic cell transfusion). In another embodiment, the disclosure provides a T lymphocyte e.g., a CD8⁺ T lymphocyte transformed/transfected by a vector or plasmid encoding a TAP-specific TCR. In a further embodiment the disclosure provides a method of treating a patient with autologous or allogenic cells transformed with a TAP-specific TCR. In certain embodiments, TCRs are expressed in primary T cells (e.g., cytotoxic T cells) by replacing an endogenous locus, e.g., an endogenous TRAC and/or TRBC locus, using, e.g., CRISPR, TALEN, zinc finger, or other targeted disruption systems.

In another embodiment, the present disclosure provides a nucleic acid encoding the above-noted TCR. In a further embodiment, the nucleic acid is present in a vector, such as the vectors described above.

In yet a further embodiment the use of a tumor antigen-specific TCR in the manufacture of autologous or allogenic cells for the treating of cancer (leukemia, such as AML) is provided.

In some embodiments, patients treated with the compositions (e.g., pharmaceutical compositions) of the disclosure are treated prior to or following treatment with allogenic stem cell transplant (ASCL), allogenic lymphocyte infusion or autologous lymphocyte infusion. Compositions of the disclosure include: allogenic T lymphocytes (e.g., CD8⁺ T lymphocyte) activated ex vivo against a TAP; allogenic or autologous APC vaccines loaded with a TAP; TAP vaccines and allogenic or autologous T lymphocytes (e.g., CD8⁺ T lymphocyte) or lymphocytes transformed with a tumor antigen-specific TCR. The method to provide T lymphocyte clones capable of recognizing a TAP according to the disclosure may be generated for and can be specifically targeted to tumor cells expressing the TAP in a subject (e.g., graft recipient), for example an ASCT and/or donor lymphocyte infusion (DLI) recipient. Hence the disclosure provides a CD8⁺ T lymphocyte encoding and expressing a T cell receptor capable of specifically recognizing or binding a TAP/MHC class I molecule complex. Said T lymphocyte (e.g., CD8⁺ T lymphocyte) may be a recombinant (engineered) or a naturally selected T lymphocyte. This specification thus provides at least two methods for producing CD8⁺ T lymphocytes of the disclosure, comprising the step of bringing undifferentiated lymphocytes into contact with a TAP/MHC class I molecule complex (typically expressed at the surface of cells, such as APCs) under conditions conducive of triggering T cell activation and expansion, which may be done in vitro or in vivo (i.e. in a patient administered with a APC vaccine wherein the APC is loaded with a TAP or in a patient treated with a TAP vaccine). Using a combination or pool of TAPs bound to MHC class I molecules, it is possible to generate a population CD8⁺ T lymphocytes capable of recognizing a plurality of TAPs. Alternatively, tumor antigen-specific or targeted T lymphocytes may be produced/generated in vitro or ex vivo by cloning one or more nucleic acids (genes) encoding a TCR (more specifically the alpha and beta chains) that specifically binds to a MHC class I molecule/TAP complex (i.e. engineered or recombinant CD8⁺ T lymphocytes). Nucleic acids encoding a TAP-specific TCR of the disclosure, may be obtained using methods known in the art from a T lymphocyte activated against a TAP ex vivo (e.g., with an APC loaded with a TAP); or from an individual exhibiting an immune response against peptide/MHC molecule complex. TAP-specific TCRs of the disclosure may be recombinantly expressed in a host cell and/or a host lymphocyte obtained from a graft recipient or graft donor, and optionally differentiated in vitro to provide cytotoxic T lymphocytes (CTLs). The nucleic acid(s) (transgene(s)) encoding the TCR alpha and beta chains may be introduced into a T cells (e.g., from a subject to be treated or another individual) using any suitable methods such as transfection (e.g., electroporation) or transduction (e.g., using viral vector) such as calcium phosphate-DNA co precipitation, DEAE-dextran-mediated transfection, polybrene-mediated transfection, electroporation, microinjection, liposome fusion, lipofection, protoplast fusion, retroviral infection, and biolistics. The engineered CD8⁺ T lymphocytes expressing a TCR specific for a TAP may be expanded in vitro using well known culturing methods.

The present disclosure provides methods for making the immune effector cells which express the TCRs as described herein. In one embodiment, the method comprises transfecting or transducing immune effector cells, e.g., immune effector cells isolated from a subject, such as a subject having a leukemia (e.g., AML), such that the immune effector cells express one or more TCR as described herein. In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express a TCR. In this regard, the immune effector cells may be cultured before or after being genetically modified (i.e., transduced or transfected to express a TCR as described herein).

Prior to in vitro manipulation or genetic modification of the immune effector cells described herein, the source of cells may be obtained from a subject. In particular, the immune effector cells for use with the TCRs as described herein comprise T cells. T cells can be obtained from a number of sources, including peripheral blood mononuclear cells (PBMCs), bone marrow, lymph nodes tissue, cord blood, thymus issue, tissue from a site of infection, ascites, pleural effusion, spleen tissue, and tumors. In certain embodiments, T cell can be obtained from a unit of blood collected from the subject using any number of techniques known to the skilled person, such as FICOLL™ separation. In one embodiment, cells from the circulating blood of an individual are obtained by apheresis. The apheresis product typically contains lymphocytes, including T cells, monocytes, granulocyte, B cells, other nucleated white blood cells, red blood cells, and platelets. In one embodiment, the cells collected by apheresis may be washed to remove the plasma fraction and to place the cells in an appropriate buffer or media for subsequent processing. In one embodiment of the invention, the cells are washed with PBS. In an alternative embodiment, the washed solution lacks calcium and may lack magnesium or may lack many if not all divalent cations. As would be appreciated by those of ordinary skill in the art, a washing step may be accomplished by methods known to those in the art, such as by using a semi-automated flow-through centrifuge. After washing, the cells may be resuspended in a variety of biocompatible buffers or other saline solution with or without buffer. In certain embodiments, the undesirable components of the apheresis sample may be removed in the cell directly resuspended culture media. In certain embodiments, T cells are isolated from peripheral blood mononuclear cells (PBMCs) by lysing the red blood cells and depleting the monocytes, for example, by centrifugation through a PERCOLL™ gradient. A specific subpopulation of T cells, such as CD28+, CD4+, CD8+, CD45RA+, and CD45RO+ T cells, can be further isolated by positive or negative selection techniques. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD8+ cells by negative selection, a monoclonal antibody cocktail typically includes antibodies to CD14, CD20, CD11b, CD16, HLA-DR, and CD4. Flow cytometry and cell sorting may also be used to isolate cell populations of interest for use in the present disclosure. PBMC may be used directly for genetic modification with the TCRs using methods as described herein. In certain embodiments, after isolation of PBMC, T lymphocytes are further isolated and in certain embodiments, both cytotoxic and helper T lymphocytes can be sorted into naive, memory, and effector T cell subpopulations either before or after genetic modification and/or expansion.

The present disclosure provides isolated immune cells such as CD8⁺ T lymphocytes that are specifically induced, activated and/or amplified (expanded) by a TAP (i.e., a TAP bound to MHC class I molecules expressed at the surface of cell), or a combination of TAPs. The present disclosure also provides a composition comprising CD8⁺ T lymphocytes capable of recognizing a TAP, or a combination thereof, according to the disclosure (i.e., one or more TAPs bound to MHC class I molecules) and said TAP(s). In another aspect, the present disclosure provides a cell population or cell culture (e.g., a CD8⁺ T lymphocyte population) enriched in CD8⁺ T lymphocytes that specifically recognize one or more MHC class I molecule/TAP complex(es) as described herein. Such enriched population may be obtained by performing an ex vivo expansion of specific T lymphocytes using cells such as APCs that express MHC class I molecules loaded with (e.g. presenting) one or more of the TAPs disclosed herein. “Enriched” as used herein means that the proportion of tumor antigen-specific CD8⁺ T lymphocytes in the population is significantly higher relative to a native population of cells, i.e. which has not been subjected to a step of ex vivo-expansion of specific T lymphocytes. In a further embodiment, the proportion of TAP-specific CD8⁺ T lymphocytes in the cell population is at least about 0.5%, for example at least about 1%, 1.5%, 2% or 3%. In some embodiments, the proportion of TAP-specific CD8⁺ T lymphocytes in the cell population is about 0.5 to about 10%, about 0.5 to about 8%, about 0.5 to about 5%, about 0.5 to about 4%, about 0.5 to about 3%, about 1% to about 5%, about 1% to about 4%, about 1% to about 3%, about 2% to about 5%, about 2% to about 4%, about 2% to about 3%, about 3% to about 5% or about 3% to about 4%. Such cell population or culture (e.g., a CD8⁺ T lymphocyte population) enriched in CD8⁺ T lymphocytes that specifically recognizes one or more MHC class I molecule/peptide (TAP) complex(es) of interest may be used in tumor antigen-based cancer immunotherapy, as detailed below. In some embodiments, the population of TAP-specific CD8⁺ T lymphocytes is further enriched, for example using affinity-based systems such as multimers of MHC class I molecule loaded (covalently or not) with the TAP(s) defined herein. Thus, the present disclosure provides a purified or isolated population of TAP-specific CD8⁺ T lymphocytes, e.g., in which the proportion of TAP-specific CD8⁺ T lymphocytes is at least about 50%, 60%, 70%, 80%, 85%, 90%, 95%, 96%, 97%, 98%, 99% or 100%.

The present disclosure further relates to a pharmaceutical composition or vaccine comprising the above-noted immune cell (CD8⁺ T lymphocytes) or population of TAP-specific CD8⁺ T lymphocytes. Such pharmaceutical composition or vaccine may comprise one or more pharmaceutically acceptable excipients and/or adjuvants, as described above.

The present disclosure further relates to the use of any TAP, nucleic acid, expression vector, T cell receptor, cell (e.g., T lymphocyte, APC), and/or composition according to the present disclosure, or any combination thereof, as a medicament or in the manufacture of a medicament. In an embodiment, the medicament is for the treatment of cancer, e.g., cancer vaccine. The present disclosure relates to any TAP, nucleic acid, expression vector, T cell receptor, cell (e.g., T lymphocyte, APC), and/or composition (e.g., vaccine composition) according to the present disclosure, or any combination thereof, for use in the treatment of cancer e.g., as a cancer vaccine. The TAP sequences identified herein may be used for the production of synthetic peptides to be used i) for in vitro priming and expansion of tumor antigen-specific T cells to be injected into tumor patients and/or ii) as vaccines to induce or boost the anti-tumor T cell response in cancer patients.

In another aspect, the present disclosure provides the use of a TAP described herein (SEQ ID NOs: 1-190, preferably SEQ ID NOs: 97-154), or a combination thereof (e.g. a peptide pool), as a vaccine for treating cancer in a subject. The present disclosure also provides the TAP described herein, or a combination thereof (e.g. a peptide pool), for use as a vaccine for treating cancer in a subject. In an embodiment, the subject is a recipient of TAP-specific CD8⁺ T lymphocytes. Accordingly, in another aspect, the present disclosure provides a method of treating cancer (e.g., of reducing the number of tumor cells, killing tumor cells), said method comprising administering (infusing) to a subject in need thereof an effective amount of CD8⁺ T lymphocytes recognizing (i.e. expressing a TCR that binds) one or more MHC class I molecule/ TAP complexes (expressed at the surface of a cell such as an APC). In an embodiment, the method further comprises administering an effective amount of the TAP, or a combination thereof, and/or a cell (e.g., an APC such as a dendritic cell) expressing MHC class I molecule(s) loaded with the TAP(s), to said subject after administration/infusion of said CD8⁺ T lymphocytes. In yet a further embodiment, the method comprises administering to a subject in need thereof a therapeutically effective amount of a dendritic cell loaded with one or more TAPs. In yet a further embodiment the method comprises administering to a patient in need thereof a therapeutically effective amount of an allogenic or autologous cell that expresses a recombinant TCR that binds to a TAP presented by an MHC class I molecule.

In another aspect, the present disclosure provides the use of CD8⁺ T lymphocytes that recognize one or more MHC class I molecules loaded with (presenting) a TAP, or a combination thereof, for treating cancer (e.g., of reducing the number of tumor cells, killing tumor cells) in a subject. In another aspect, the present disclosure provides the use of CD8⁺ T lymphocytes that recognize one or more MHC class I molecules loaded with (presenting) a TAP, or a combination thereof, for the preparation/manufacture of a medicament for treating cancer (e.g., for reducing the number of tumor cells, killing tumor cells) in a subject. In another aspect, the present disclosure provides CD8⁺ T lymphocytes (cytotoxic T lymphocytes) that recognize one or more MHC class I molecule(s) loaded with (presenting) a TAP, or a combination thereof, for use in the treatment of cancer (e.g., for reducing the number of tumor cells, killing tumor cells) in a subject. In a further embodiment, the use further comprises the use of an effective amount of a TAP (or a combination thereof), and/or of a cell (e.g., an APC) that expresses one or more MHC class I molecule(s) loaded with (presenting) a TAP, after the use of said TAP-specific CD8⁺ T lymphocytes.

The present disclosure also provides a method of generating an immune response against tumor cells (leukemic cells, AML cells) expressing human class I MHC molecules loaded with any of the TAP disclosed herein or combination thereof in a subject, the method comprising administering cytotoxic T lymphocytes that specifically recognizes the class I MHC molecules loaded with the TAP or combination of TAPs. The present disclosure also provides the use of cytotoxic T lymphocytes that specifically recognizes class I MHC molecules loaded with any of the TAP or combination of TAPs disclosed herein for generating an immune response against tumor cells expressing the human class I MHC molecules loaded with the TAP or combination thereof.

In an embodiment, the methods or uses described herein further comprise determining the HLA class I alleles expressed by the patient prior to the treatment/use, and administering or using TAPs that bind to one or more of the HLA class I alleles expressed by the patient. For example, if it is determined that the patient expresses HLA-A1*01 and HLA-C05*01, any combinations of the TAPs of (i) SEQ ID NOs: 48, 67, 89, 134, 151 and/or 164 (that bind to HLA-A1*01), and/or SEQ ID NO: 150 (that binds to HLA-C05*01) may be administered or used in the patient.

In an embodiment, the cancer is a blood cancer, preferably leukemia such as acute lymphocytic leukemia (ALL), acute myeloid leukemia (AML), chronic myeloid leukemia (CML), hairy cell leukemia (HCL) and myelodysplastic syndromes (MDS). In an embodiment, the leukemia is AML. The AML treated by the methods and uses described herein may be of any type or subtype (e.g., low-, intermediate- or high-risk AML), for example AML with genetic abnormalities such as AML with a translocation between chromosomes 8 and 21 [t(8;21)], AML with a translocation or inversion in chromosome 16 [t(16;16) or inv(16)], AML with the PML-RARA fusion gene, AML with a translocation between chromosomes 9 and 11 [t(9;11)], AML with a translocation between chromosomes 6 and 9 [t(6:9)], AML with a translocation or inversion in chromosome 3 [t(3;3) or inv(3)], AML (megakaryoblastic) with a translocation between chromosomes 1 and 22 [t(1:22)], AML with the BCR-ABL1 (BCR-ABL) fusion gene, AML with mutated NPM1 gene, AML with biallelic mutations of the CEBPA gene, AML with mutated RUNX1 gene, AML with mutated ASX1 gene, AML with mutated IDH1 and/or IDH2 gene, AML with mutated FLT3 gene, AML with myelodysplasia-related changes, and AML related to previous chemotherapy or radiation.

In an embodiment, the TAP, nucleic acid, expression vector, T cell receptor, cell (e.g., T lymphocyte, APC), and/or composition according to the present disclosure, or any combination thereof, may be used in combination with one or more additional active agents or therapies to treat cancer, such as chemotherapy (e.g., vinca alkaloids, agents that disrupt microtubule formation (such as colchicines and its derivatives), anti-angiogenic agents, therapeutic antibodies, EGFR targeting agents, tyrosine kinase targeting agent (such as tyrosine kinase inhibitors), transitional metal complexes, proteasome inhibitors, antimetabolites (such as nucleoside analogs), alkylating agents, platinum-based agents, anthracycline antibiotics, topoisomerase inhibitors, macrolides, retinoids (such as all-trans retinoic acids or a derivatives thereof), geldanamycin or a derivative thereof (such as 17-AAG), surgery, radiotherapy, immune checkpoint inhibitors (immunotherapeutic agents (e.g., PD-1/PD-L1 inhibitors such as anti-PD-1/PD-L1 antibodies, CTLA-4 inhibitors such as anti-CTLA-4 antibodies, B7-1/B7-2 inhibitors such as anti-B7-1/B7-2 antibodies, TIM3 inhibitors such as anti-TIM3 antibodies, BTLA inhibitors such as anti-BTLA antibodies, CD47 inhibitors such as anti-CD47 antibodies, GITR inhibitors such as anti-GITR antibodies), antibodies against tumor antigens, cell-based therapies (e.g., CAR T cells, CAR NK cells), cytokines such as IL-2, IL-7, IL-21, and IL-15. In an embodiment, the TAP, nucleic acid, expression vector, T cell receptor, cell (e.g., T lymphocyte, APC), and/or composition according to the present disclosure is administered/used in combination with an immune checkpoint inhibitor. In an embodiment, the TAP, nucleic acid, expression vector, T cell receptor, cell (e.g., T lymphocyte, APC), and/or composition according to the present disclosure is administered/used in combination one or more chemotherapeutic drugs used for the treatment of AML, or in combination with other AML therapy, for example stem cell/bone marrow transplantation.

The additional therapy may be administered prior to, concurrent with, or after the administration of the TAP, nucleic acid, expression vector, T cell receptor, cell (e.g., T lymphocyte, APC), and/or composition according to the present disclosure.

### MODE(S) FOR CARRYING OUT THE INVENTION

The present invention is illustrated in further details by the following non-limiting examples.

### Example 1: Materials and Methods

### AML Specimens

Diagnostic AML samples (cryovials of DMSO-frozen leukemic blasts) were obtained from the Banque de cellules leucémiques du Québec program (BCLQ, bclq.org). Samples technical and clinical characteristics are provided in Table 1. One hundred million cells of each AML sample (except 14H124, see section below) were thawed (1 min in 37° C. water bath) and resuspended in 48 ml of 4° C. PBS. Two million cells (1 ml) were pelleted and resuspended in 1 ml Trizol for RNA-Sequencing while the remaining 98 million were pelleted and snap-frozen in liquid nitrogen for mass spectrometry analyses.

TABLE 1

Biological and clinical characteristics of the 19 AML specimens used to identify TSAs in the present study

| ID BCLQ | HLA typing (optitype) | WHO 2008 | FAB | Tissue | % blasts | RNA-seq read count |
|---|---|---|---|---|---|---|
| 05H143 | A*02:01, A*34:02, B*08:01, B*57:01, C*07:01, C*06:02 | Acute monoblastic and monocytic leukaemia | M5A | Peripheral blood | 85 | 2.14E+08 |
| 05H149 | A*24:02, A*68:02, B*53:01, B*18:01, C*04:01, C*05:01 | Acute myeloid leukaemia, NOS | M1 | Peripheral blood | 80 | 1.19E+08 |
| 07H060 | A*01:01, A*29:02, B*08:01, B*56:01, C*07:01, C*01:02 | AML without maturation | M1 | Peripheral blood | 90 | 2.04E+08 |
| 07H063 | A*30:01, A*01:01, B*13:02, B*07:02, C*06:02, C*07:02 | Therapy-related myeloid neoplasms | M5A | Bone marrow | 96 | 1.92E+08 |
| 07H122 | A*02:01, A*02:01, B*57:01, B*57:01, C*06:02, C*06:02 | AML with myelodysplasia- related changes | NC | Peripheral blood | 96 | 1.84E+08 |
| 07H141 | A*02:01, A*26:01, B*38:01, B*44:02, C*05:01, C*12:03 | AML without maturation | M1 | Peripheral blood | 75 | 1.77E+08 |
| 08H039 | A*02:01, A*01:01, B*40:01, B*14:02, C*03:04, C*08:02 | AML with minimal differentiation | M0 | Peripheral blood | 96 | 1.82E+08 |
| 08H053 | A*01:01, A*11:01, B*14:02, B*15:01, C*03:03, C*03:03 | AML without maturation | M1 | Peripheral blood | 95 | 1.74E+08 |
| 10H005 | A*11:01, A*11:01, B*13:01, B*13:01, C*07:02, C*03:04 | Acute myelomonocytic leukaemia | M4 | Bone marrow | 71 | 1.84E+08 |
| 11H008 | A*29:02, A*03:01, B*44:03, B*57:01, C*06:02, C*16:01 | AML with myelodysplasia- related changes | NC | Peripheral blood | 75 | 1.76E+08 |
| 11H035 | A*03:01, A*24:02, B*07:02, B*07:02, C*07:02, C*07:02 | AML without maturation | M1 | Peripheral blood | 93 | 1.80E+08 |
| 12H172 | A*24:02, A*02:01, B*15:01, B*15:01, C*03:03, C*03:03 | Therapy-related myeloid neoplasms | M1 | Peripheral blood | 85 | 1.91E+08 |
| 14H124 | A*02:01, A*26:01, B*44:05, B*27:05, C*02:02, C*02:02 | AML with maturation | M2 | Peripheral blood | 86 | 1.23E+08 |
| 15H013 | A*30:02, A*02:01, B*39:05, B*57:03, C*18:01, C*07:02 | AML without maturation | M1 | Peripheral blood | 90 | 1.61E+08 |
| 15H023 | A*02:01, A*03:01, B*51:01, B*44:03, C*01:02, C*02:02 | AML without maturation | M1 | Peripheral blood | 90 | 1.79E+08 |
| 15H063 | A*02:01, A*01:01, B*08:01, B*40:01, C*07:01, C*03:04 | AML with myelodysplasia-related changes | NC | Peripheral blood | 85 | 1.65E+08 |
| 15H080 | A*02:01, A*26:01, B*44:05, B*27:05, C*02:02, C*02:02 | Acute monoblastic and monocytic leukaemia | M5A | Peripheral blood | 70 | 1.95E+08 |
| 16H123 | A*24:02, A*03:01, B*15:01, B*07:02, C*07:02, C*03:03 | AML without maturation | M1 | Peripheral blood | 88 | 1.66E+08 |
| 16H145 | A*02:01, A*02:01, B*27:02, B*55:01, C*03:03, C*02:02 | AML with minimal differentiation | M0 | Peripheral blood | 88 | 1.85E+08 |

| ID BCLQ | Unique k-mer (33) count | Occurrence threshold used in mTECs depletion | K-mer count after occurence filtering | K-mer count after mTECs filtering | Occurrence threshold used in mTECs+ MPCs depletion | K-mer count after occurrence filtering |
|---|---|---|---|---|---|---|
| 05H143 | 1.44E+09 | 10 | 1.26E+08 | 3.01E+07 | 3 | 3.38E+08 |
| 05H149 | 8.89E+08 | 12 | 7.64E+07 | 2.33E+07 | 5 | 1.78E+08 |
| 07H060 | 1.24E+09 | 8 | 1.11E+08 | 2.02E+07 | 3 | 2.76E+08 |
| 07H063 | 9.55E+08 | 6 | 9.26E+07 | 1.62E+07 | 3 | 1.80E+08 |
| 07H122 | 9.67E+08 | 7 | 8.35E+07 | 1.85E+07 | 3 | 2.03E+08 |
| 07H141 | 1.08E+09 | 10 | 8.81E+07 | 1.55E+07 | 3 | 2.55E+08 |
| 08H039 | 1.10E+09 | 8 | 9.72E+07 | 1.85E+07 | 3 | 2.47E+08 |
| 08H053 | 1.14E+09 | 8 | 9.08E+07 | 1.56E+07 | 3 | 2.40E+08 |
| 10H005 | 1.01E+09 | 6 | 8.63E+07 | 1.56E+07 | 3 | 1.81E+08 |
| 11H008 | 8.30E+08 | 5 | 9.73E+07 | 1.79E+07 | 3 | 1.68E+08 |
| 11H035 | 1.07E+09 | 7 | 1.06E+08 | 1.86E+07 | 3 | 2.39E+08 |
| 12H172 | 1.12E+09 | 6 | 1.07E+08 | 2.17E+07 | 3 | 2.18E+08 |
| 14H124 | 7.77E+08 | 4 | 1.03E+08 | 1.76E+07 | 3 | 1.35E+08 |
| 15H013 | 8.65E+08 | 5 | 1.03E+08 | 1.86E+07 | 3 | 1.73E+08 |
| 15H023 | 1.21E+09 | 8 | 1.12E+08 | 2.28E+07 | 3 | 2.75E+08 |
| 15H063 | 1.02E+09 | 8 | 9.79E+07 | 2.16E+07 | 3 | 2.29E+08 |
| 15H080 | 1.13E+09 | 7 | 1.11E+08 | 2.23E+07 | 3 | 2.44E+08 |
| 16H123 | 1.06E+09 | 8 | 1.01E+08 | 1.77E+07 | 3 | 2.50E+08 |
| 16H145 | 1.19E+09 | 8 | 1.08E+08 | 2.12E+07 | 3 | 2.65E+08 |

| ID BCLQ | K-mer count after mTECs + MPCs filtering | Distinct MAP count | | | |
|---|---|---|---|---|---|
| | | mTECs depl. | ERE | mTECs+ MPCs depl. | Diff. k-mer exp. |
| 05H143 | 2.53 E+07 | 2894 | 2358 | 2897 | 2877 |
| 05H149 | 2.47 E+07 | 542 | 519 | 545 | 539 |
| 07H060 | 1.83 E+07 | 986 | 957 | 983 | 973 |
| 07H063 | 8.14 E+06 | 1784 | 1777 | 1797 | 1715 |
| 07H122 | 2.53 E+07 | 2940 | 2702 | 2911 | 2878 |
| 07H141 | 1.37 E+07 | 1662 | 1554 | 1647 | 1544 |
| 08H039 | 2.27 E+07 | 2594 | 2420 | 2689 | 2670 |
| 08H053 | 1.81 E+07 | 317 | 270 | 328 | 292 |
| 10H005 | 1.06 E+07 | 220 | 136 | 280 | 159 |
| 11H008 | 9.91 E+06 | 1163 | 1054 | 1175 | 1072 |
| 11H035 | 1.53 E+07 | 1928 | 1899 | 1986 | 1933 |
| 12H172 | 1.59 E+07 | 2865 | 2916 | 2861 | 2851 |
| 14H124 | 3.63 E+06 | 2160 | 2093 | 2249 | 2116 |
| 15H013 | 8.86 E+06 | 1761 | 1686 | 1756 | 1748 |
| 15H023 | 1.68 E+07 | 1794 | 1733 | 1799 | 1732 |
| 15H063 | 1.25 E+07 | 1277 | 1128 | 1285 | 1190 |
| 15H080 | 1.23 E+07 | 797 | 722 | 796 | 760 |
| 16H123 | 1.80 E+07 | 1424 | 1383 | 1467 | 1391 |
| 16H145 | 1.71 E+07 | 755 | 704 | 765 | 742 |

NC: not classifiable by FAB criteria. HLA were determined by optitype based on RNA-Seq data of each sample. Clinical data have been provided by the Banque de cellules leucémiques duQuébec program (BCLQ, bclq.org).

### Other Sources of Data

Human mTEC samples have either been prepared and sequenced for the need of previous studies of our team (#GSE127825 and # GSE127826) (Larouche et al., 2020; Laumont et al., 2018) or have been published by others (E-MTAB-7383) (Fergusson et al., 2018). Only the six mTEC samples previously used for TSA discovery by our group have been used in k-mer depletion approach (Laumont et al., 2018). The 11 MPCs samples used as main normal controls have been sequenced by the IRIC genomic platform and published previously by the Leucegene group (#GSE98310, #GSE51984). All other normal samples used in the present study have been downloaded from dbGap (www.ncbi.nlm.nih.gov/gap/), Arrayexpress (www.ebi.ac.uk/arrayexpress/) or GEO (www.ncbi.nlm.nih.gov/geo/). The Leucegene full cohort of 437 RNA-sequenced AML samples was used to study the clinical significance of discovered TSAs^{hi}. RNA-sequencing data have been published previously and are available separately (#GSE49642, #GSE52656, #GSE62190, #GSE66917, #GSE67039) (Lavallee et al., 2015; Macrae et al., 2013; Pabst et al., 2016). RNA-Seq data of sorted LSC and blasts were published elsewhere and obtained from #GSE74246 (Corces et al., 2016). RNA-seq data of AML blasts pre-and post-relapse (matched samples) were published elsewhere (Toffalori et al., 2019) and HLA types of these samples were kindly provided by Dr. Luca Vago. All data obtained from external sources were aligned on GRCh38 genome with STAR v2.5.1b.

### Expansion of 14H124 AML Cells in NSG Mice

Because only 20 million cells were available for this patient, blasts of patient 14H124 have been thawed, washed in PBS, and intravenously injected to 10 NOD-scid IL-2Rγnull ^{(NSG)} mice (2 × 10⁶ / mouse), 24 h following sub-lethal total body irradiation (2.5 Gy, 137 Cs-gamma source). Human AML cell engraftment was assessed in peripheral blood at day 122 by flow cytometry. Briefly, 100 µl of blood were collected by tail vein bleeding, depleted of erythrocytes using RBC lysis buffer (eBioscience), washed in staining buffer (PBS+3%FBS), stained with antihuman CD45-PacificBlue (Hl30, Biolegend) and anti-mouse CD45-PECy5 (30-F11, BD) for 20 min at 4° C. and washed in PBS. Data were acquired on a FACS Canto II flow cytometer (Becton Dickinson) and analyzed with the Flowjo® software 7.0 (Tree Star Inc., Ashland, OR).

Human cell chimerism higher than 1% was found in 8/10 mice. Mice were sacrificed within days 188-264 post-transplantation at signs of disease (anemia, weight loss >20% or apparent tumor) and bone marrow, spleen and solid tumors (found in interscapular, neck and hips regions or in kidneys, liver and lymph nodes) were harvested. Tumors were snap-frozen in liquid nitrogen for future processing by mass spectrometry. AML cells were collected by crushing the spleens and flushing the femurs and tibiae (collection of bone marrow) with 4° C. PBS. Cells were depleted of erythrocytes, filtered (100 µm) to remove debris, counted and either processed for flow cytometry (5×10⁵ cells) as detailed above to assess their purity or were lysed and cryopreserved in Trizol® (Invitrogen) for future RNA sequencing (all remaining cells). One tumor having a size >1 cm³ and for which 6 million bone marrow-derived blasts of purity >99% (FIGS. 14A-B) were available for RNA sequencing was chosen to be processed for MAPs identification by mass spectrometry. The two mice having no human chimerism (graft failure) at day 122 in peripheral blood did not develop any sign of disease and were sacrificed at end of experiment (day 264). All sacrifices were performed humanely by CO₂ asphyxiation followed by cervical dislocation. Mice were assessed for disease signs thrice weekly and monitored daily during the experiment.

### RNA Extraction, Library Preparation and Sequencing

RNA extractions have been done using Trizol®/chlorophorm extraction and purification on RNeasy® Mini extraction columns (Qiagen). 400 ng of total RNA was used for library preparation. Quality of total RNA was assessed with the BioAnalyzer Nano (Agilent) and all samples had a RIN above 8. Library preparation was done with the KAPA mRNAseq Hyperprep kit (KAPA, Cat no. KK8581). Ligation was made with 51 nM final concentration of Illumina Truseq index and 12 PCR cycles was required to amplify cDNA libraries. Sample 14H124 was done separately using 4 M cells, 1 ug of total RNA. Library preparation was performed like previous samples except the amplification was done with 10 PCR cycles instead of 12. Libraries were quantified by QuBit and BioAnalyzer DNA1000. All libraries were diluted to 10 nM and normalized by qPCR using the KAPA library quantification kit (KAPA; Cat no. KK4973). Libraries were pooled to equimolar concentration. Sequencing was performed with the Illumina Nextseq500 using the Nextseq High Output Kit 150 cycles (2x80 bp) using 2.8 pM of the pooled libraries. Around 120-200 M paired-end PF reads was generated per sample. Library preparation and sequencing was made at the Institute for Research in Immunology and Cancer’s Genomics Platform (IRIC).

### Database Generation for Shotgun Mass Spectrometry Identifications

1) Generation of personalized canonical proteomes. This was conducted as detailed previously (Laumont et al., 2018). Briefly, RNA-Seq reads were trimmed using Trimmomaticv0.35 and aligned to GRCh38.88 using STAR v2.5.1b (Dobin et al., 2013) running with default parameters except for --alignSJoverhangMin, --alignMatesGapMax, --alignlntronMax, and --alignSJstitchMismatchNmax parameters for which default values were replaced by 10, 200,000, 200,000 and “5-1 5 5”, respectively, to generate bam files. Single-base mutations with a minimum alternate count setting of 5 were identified using freeBayes 1.0.2-16-gd466dde (arXiv:1207.3907). Transcript expression was quantified in transcripts per million (tpm) with kallisto v0.43.0 (Bray et al., 2016) with default parameters. Finally, pyGeno was used to insert high-quality sample-specific single-base mutations (freeBayes quality >20) in the reference exome and export sample-specific sequences of known proteins generated by expressed transcripts (tpm > 0) to generate fasta files of personalized canonical proteomes.

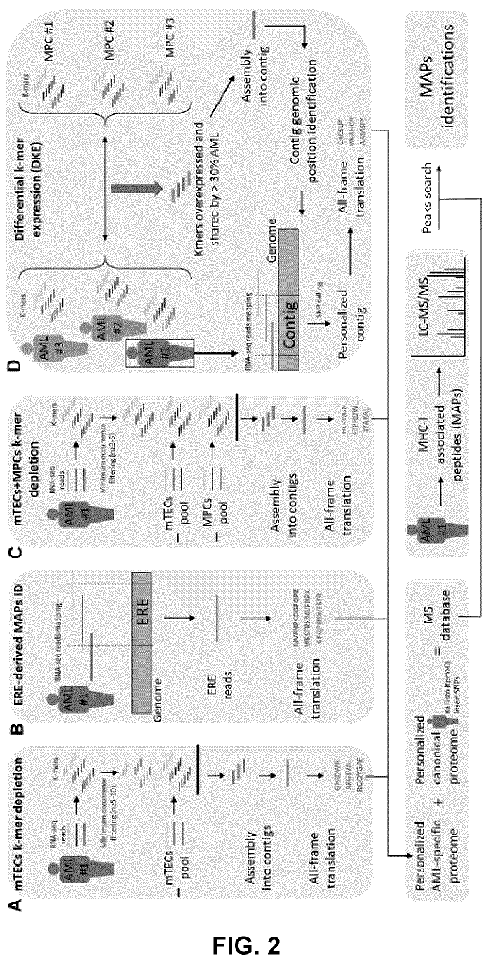

2) Generation of AML-specific proteome by mTECs k-mer depletion (FIG. 2A). This was conducted as detailed previously (Laumont et al., 2018). Briefly, R1 and R2 fastq files of each sample were trimmed as reported above and R1 reads were reverse complemented using the fastx_reverse_complement function of the FASTX-Toolkit v0.0.14. K-mer databases (24 or 33-long) were generated with Jellyfish v2.2.3 (Marcais and Kingsford, 2011). A single database was generated for each AML sample while the 6 mTEC samples were combined in a unique database by concatenating their fastq files. Because the duration of k-mer assembly (see hereafter) increases exponentially above 30 million k-mers, each AML 33-nucleotide-long k-mer database was filtered based on a sample-specific threshold on occurrence (the number of times that a given k-mer is present in the database) in order to reach a maximum of 30 million k-mers for the assembly step (Table 1). After this filtering, k-mers present at least once in the mTECs k-mer database were removed from each sample database and remaining k-mers were assembled into contigs with NEKTAR, an in-house developed software. Briefly, one of the submitted 33-nucleotide-long k-mer is randomly selected as a seed that is extended from both ends with consecutive k-mers overlapping by 32 nucleotides on the same strand (-r option disabled, as stranded sets of k-mers were used). The assembly process stops when either no k-mers can be assembled or when more than one k-mer fits (-a 1 option for linear assembly). If so, a new seed is selected and the assembly process resumes until all k-mers from the submitted list have been used once. Finally, the contigs were 3-frame translated using an in-house python script, amino acid sequences were split at internal stop codons and the resulting subsequences were concatenated with each sample respective personalized canonical proteome.

3) Generation of ERE-specific proteome (FIG. 2B). For each sample, RNA-Seq reads were aligned on the human reference genome (GRCh38.88) using STAR (Dobin et al., 2013) with default parameters. Using the intersect function of BEDtools (PMID 20110278), reads were separated in two datasets of reads entirely mapping in either ERE sequences or canonical genes. Reads of the ERE reads dataset were discarded if their sequences were also present in the canonical reads dataset. Unmapped reads, secondary alignments and low-quality reads were then discarded from the ERE reads dataset with samtools view (PMID 19505943). Remaining ERE reads were then in silico translated into ERE polypeptides in all possible reading frames. ERE polypeptides were spliced at the location of stop codons, downstream sequences were discarded and only upstream sequences of ≥8 amino acids (i.e. the minimal length of a MAP) were kept. The resulting ERE proteome was then concatenated with the respective sample’s personalized canonical proteome.

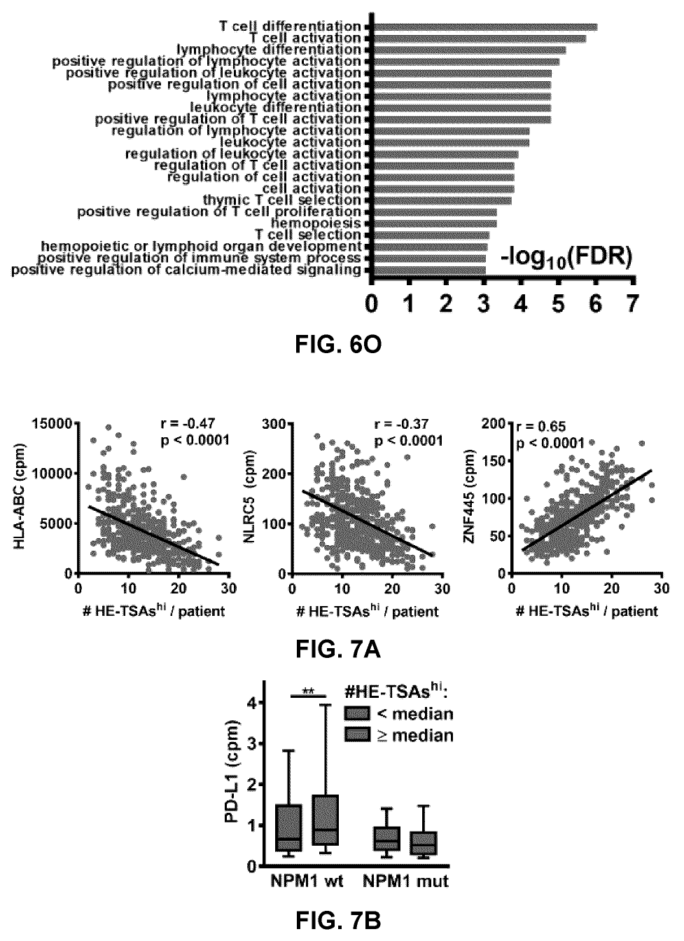

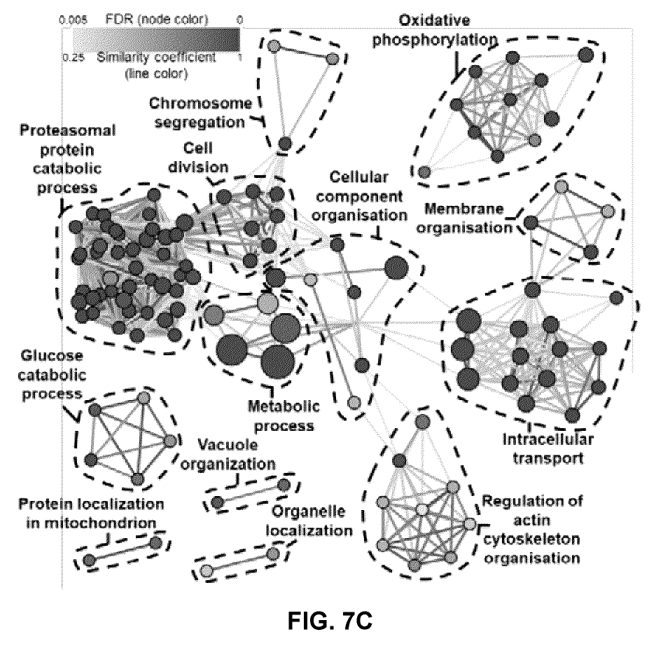

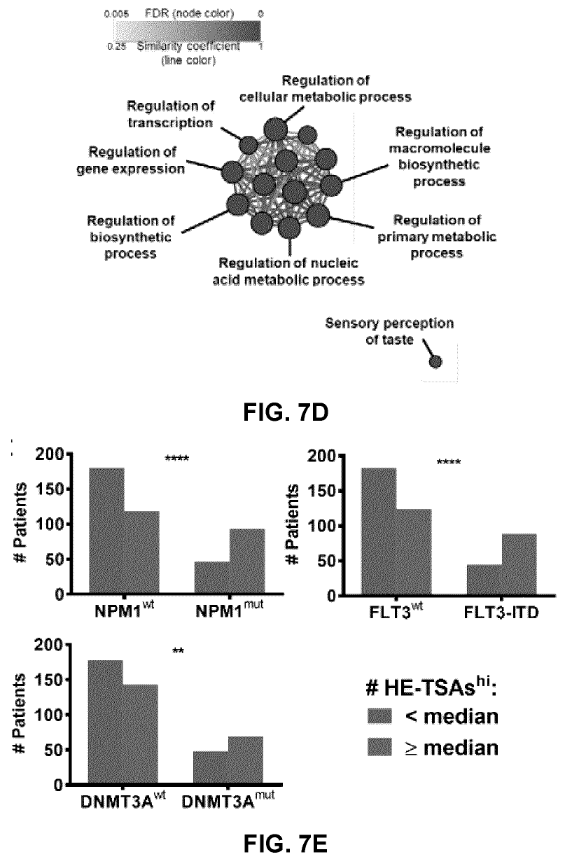

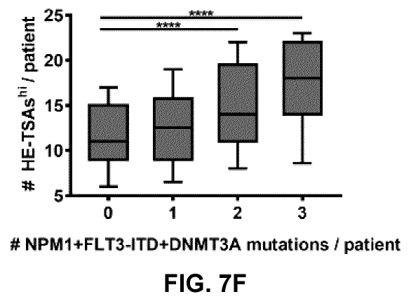

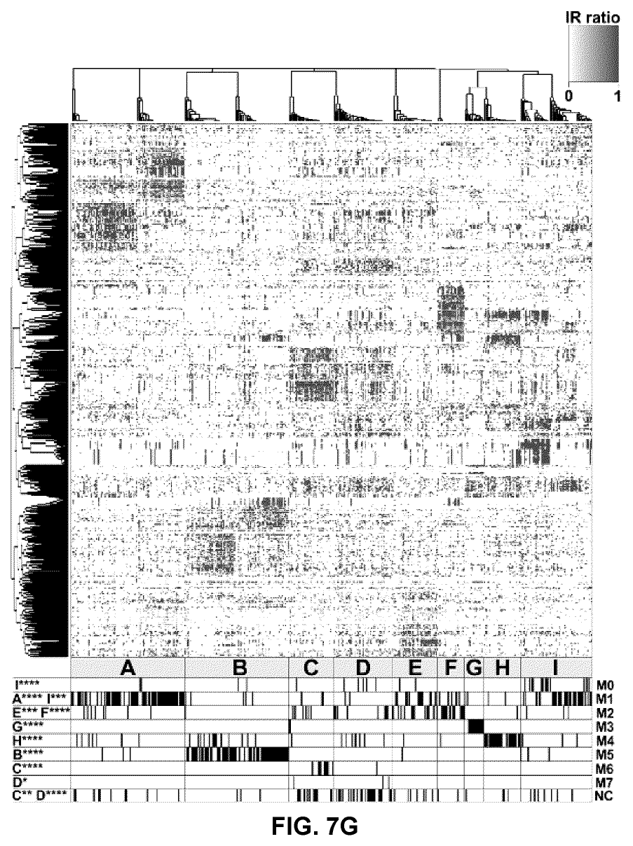

4) Generation of AML-specific proteome by mTECs+MPCs k-mer depletion (FIG. 2C). To perform this approach, the same methods described for mTECs k-mer depletion were used with the following modifications: (i) an additional normal k-mer database, combining the fastq files of 11 MPC samples used as k-mer controls, was generated with Jellyfish. Because these samples were not sequenced in stranded mode, the k-mer database was generated with the -C option and R1 fastq files were not reverse-complemented. (ii) AML k-mers present either in mTECs or MPCs k-mer database were removed, and consequently the number of k-mers filtered by this step was greater than in mTECs k-mer depletion approach. (iii) Because of the higher efficacy of k-mer depletion by normal samples, it was possible to use lower occurrence thresholds to pre-filterAML k-mers (Table 1 and FIG. 7C), changing dramatically the identity of k-mers present in these databases compared to mTECs k-mers depletion only (FIG. 7D). Importantly, no occurrence threshold lower than 3 was used in order to exclude possible sequencing errors. All other procedures were conducted as reported in “Generation of AML-specific proteome by mTECs k-mer depletion” section.

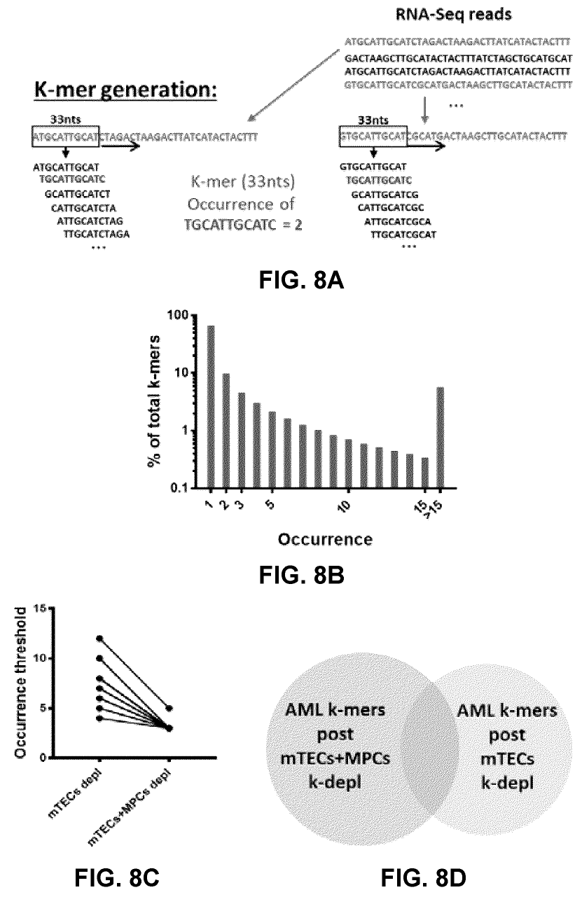

5) Generation of AML-specificproteome by differential k-mer expression (FIG. 2D). The differential k-mer analysis has been performed based on a customized use of DE-kupl, a computational pipeline performing the generation of k-mer databases from fastq files, the normalization of k-mer abundances, the filtering of k-mers based on their occurrence and their inter-sample sharing, the comparison of k-mer abundance between samples in two different conditions through the use of statistical tests, the assembly of differentially expressed k-mers into contigs, the alignment of contigs on the genome and the annotation of contigs based on their genomic alignment (FIG. 8) (Audoux et al., 2017). Specifically, a DE-kupl run was first performed with the following parameters diff_method Ttest, kmer_length 33, gene_diff_method limma-voom, data_type WGS, lib_type unstranded, min_recurrence 6, min_recurrence_abundance 3, pvalue_threshold 0.05 and log2fc_threshold 0.1 to compare the AML specimens to the 11 MPC controls. This returned a diff-counts.tsv file containing the sequence and normalized counts of significantly differentially expressed 33-nucleotide-long k-mers (FDR<0.05) between AML and MPC samples, and present at a minimal occurrence of 3 in at least 6 samples (either MPC or AML). Because custom rules of k-mer filtering were desired, no restriction was applied on k-mers fold changes in DE-kupl (log2fc_threshold 0.1) and the k-mer list provided in the diff-counts.tsv file were rather manually filtered to keep all k-mers (i) fully absent (count=0) in all MPC samples (and therefore present in at least 6 AML samples); OR (ii) present in at least 6 AML samples (>30% of the specimens) and having a fold change ≥10-fold; OR (iii) present in a single MPC sample, with an abundance lower than the lowest abundance in AML samples; OR (iv) present in at least 6 AML samples, with a fold change ≥5-fold and a FDR≤ 0.000001. Based on these rules, 6 a new diff-counts.tsv file containing ~41×10⁶ k-mers was generated and used to perform k-mer 6 assembly through DE-kupl, and a merged-diff-counts.tsv file containing ~2.1×10⁶ contigs was obtained. Finally, the annot function of DE-kupl was used to map and annotate the generated contigs on GRCh38 human genome.

To obtain personalized contig sequences for each AML sample, the DiffContigslnfos.tsv output of DE-kupl annot was used to build a bed file of all contigs having a length ≥34 nucleotides (deriving from the assembly of at least 2 k-mers) and which aligned without gaps, insertions or deletions (CIGAR without N/D/I). Next, this bed file and the bedtools, samtools and bcftools suites were used to extract personalized contig sequences (bedtools getfasta -fi consensus.fasta -bed contigs.bed -name >> output.fasta) from a consensus genome generated from the bam file (reads mapped on GRCh38 with star, see “Generation of personalized canonical proteomes.” section) of each AML sample (samtools mpileup -C50 -uf ref_genome.fasta sample.bam | bcftools call -c | vcfutils.pl vcf2fq -d 8 -D 100 | awk ‘/^@chr.$|^chr..$|^@GL........$|^@KI........$/,/^+$/’ | sed ‘/^+/d’ | tr “@” “>” > consensus.fasta).

Portions of contigs not covered by reads (N’s) were removed with sed (sed -E “s/NNN+/\n/g”) and all contigs were written in a fasta file. Sequences of contigs having alignments with gaps, insertions or deletions (and which cannot be retrieved from a consensus genome) and which were reported as expressed by the relevant sample in DiffContigslnfos.tsv were added to this fasta file. Finally, by using in-house python scripts (published previously or being included in pyGeno (Daouda et al., 2016; Laumont et al., 2018)), the contigs were 6-frame translated, ambiguous amino acid sequences were transformed into all possible sequences (as contigs overlapping single base mutations can code for multiple different amino acid sequences), amino acid sequences were split at internal stop codons and the resulting subsequences were concatenated with each sample respective personalized canonical proteome.

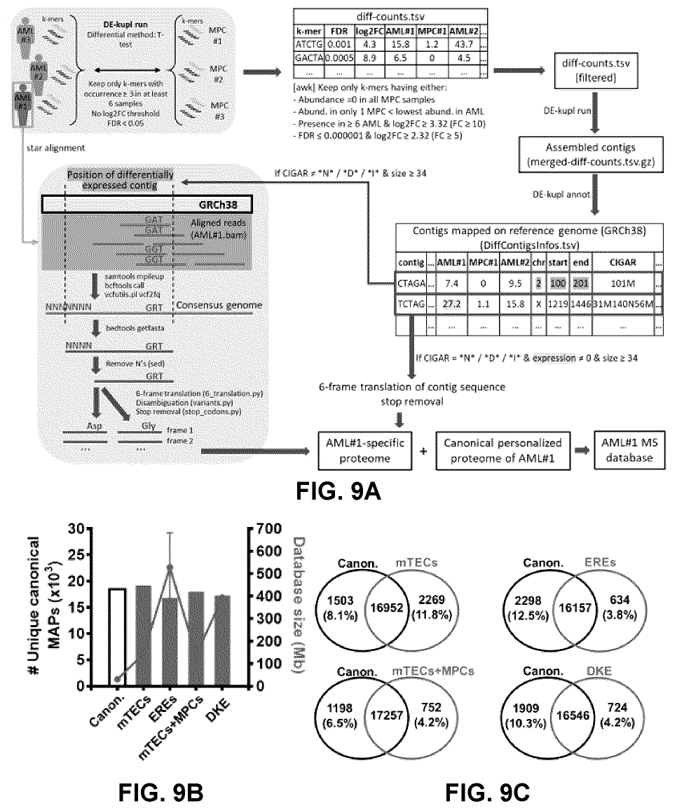

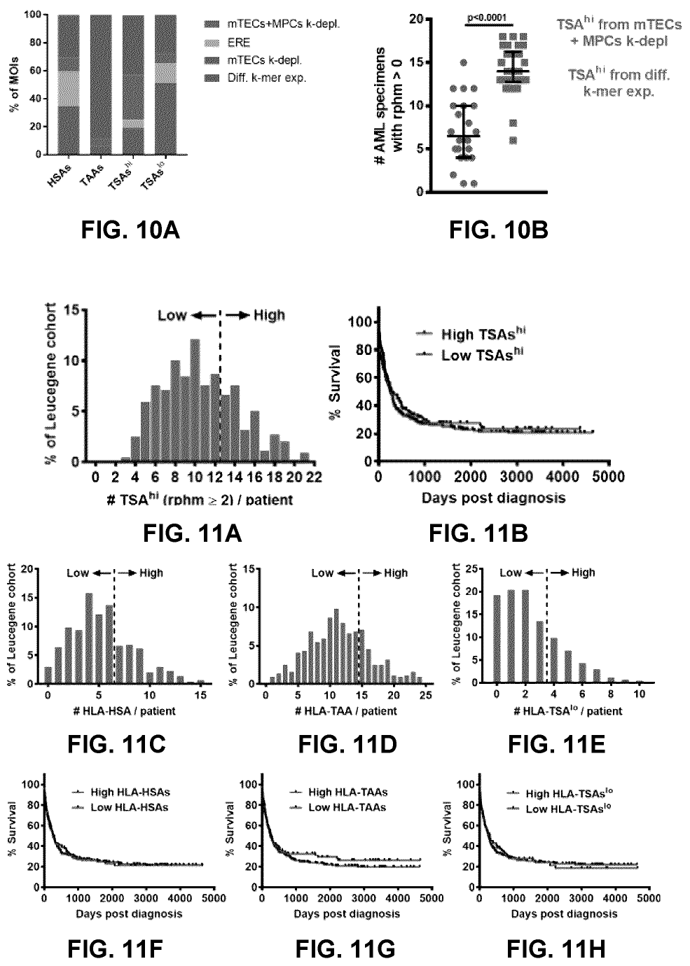

6) Validation of database size - FIGS. 9B-C. Given that MS databases used in the four proteogenomic approaches used in this study presented variably inflated sizes compared to the canonical (personalized) proteome databases, how these higher sizes affected MS identifications was examined. First, the cumulative number of peptides identified with each approach was compared across the 19 AML samples (FIG. 9B). This showed that despite significant differences in database size between the approaches, the number of peptides identified varied only modestly (up to ~9% for ERE approach) compared to the canonical proteome. Next, as each database were concatenated with the respective canonical personalized proteome in each sample, it was reasoned that databases of appropriate size should allow the identification of canonical protein-derived peptides of similar identity vs. the canonical proteome alone. As showed in FIG. 9C, the vast majority (88.2% - 96.2%) of peptides annotated as protein-coding identified in each approach across all AML samples were common with those identified based on the canonical proteome alone. Based on these observations, it was concluded that the size of the various databases was suitable for reliable MS identifications.

### Isolation of MHC-Associated Peptides

The W6/32 antibodies (BioXcell) were incubated in PBS for 60 minutes at room temperature with PureProteome protein A magnetic beads (Millipore) at a ratio of 1 mg of antibody per mL of slurry. Antibodies were covalently cross-linked to magnetic beads using dimethylpimelidate as described. The beads were stored at 4° C. in PBS pH 7.2 and 0.02% NaN3. For frozen cell pellet samples (98 million cells/pellet), cells were thawed and resuspended in 1 mL PBS pH 7.2 and solubilized by adding 1 mL of detergent buffer containing PBS pH 7.2, 1% (w/v) CHAPS (Sigma) supplemented with Protease inhibitor cocktail (Sigma). For tumor sample, the tissue was cut into small pieces (cubes, ~3 mm in size) and 5 ml of ice-cold PBS containing Protease inhibitor cocktail was added. Tissue pieces were first homogenized twice for 20 seconds using an Ultra Turrax T25 homogenizer (IKA-Labortechnik) set at speed 20000 rpm and then 20 seconds using an Ultra Turrax T8 homogenizer (IKA-Labortechnik) set at speed 25000 rpm. Then, 550 µl of ice-cold 10X lysis buffer (5% w/v CHAPS) was added to the sample. Cell pellet and tumor samples were incubated 60 minutes with tumbling at 4° C. and then spun at 10000 g for 20 minutes at 4° C. Supernatants were transferred into new tubes containing 1 mg of W6/32 antibody covalently-cross-linked protein A magnetic beads per sample and incubated with tumbling for 180 minutes at 4° C. Samples were placed on a magnet to recover bound MHC I complexes to magnetic beads. Magnetic beads were first washed with 8 × 1 mL PBS, then with 1 × 1 mL of 0.1X PBS and finally with 1 × 1 mL of water. MHC | complexes were eluted from the magnetic beads by acidic treatment using 0.2% formic acid (FA). To remove any residual magnetic beads, eluates were transferred into 2.0 mL Costar mL Spin-X centrifuge tube filters (0.45 µm, corning) and spun 2 minutes at 855 g. Filtrates containing peptides were separated from MHC I subunits (HLA molecules and β-2 macroglobulin) using home-made stage tips packed with twenty 1 mm diameter octadecyl (C-18) solid phase extraction disks (EMPORE). Stage tips were pre-washed first with methanol then with 80% acetonitrile (ACN) in 0.2% trifluoroacetic acid (TFA) and finally with 0.2% FA. Samples were loaded onto the stage tips and washed with 0.2% FA. Peptides were eluted with 30% ACN in 0.1%TFA, dried using vacuum centrifugation and then stored at -20° C. until MS analysis.

### Mass Spectrometry Analyses

Dried peptide extracts were resuspended in 4% formic acid and loaded on a homemade C18 analytical column (15 cm x 150 µm i.d. packed with C18 Jupiter Phenomenex) with a 56-min gradient (10H005) or 106-minute gradient (all other samples) from 0% to 30% acetonitrile (0.2% formic acid) and a 600 nL/min flow rate on an EasynLC II system. Samples were analyzed with a Q-Exactive HF mass spectrometer (Thermo Fisher Scientific) in positive ion mode with Nanospray 2 source at 1.6 kV. Each full MS spectrum, acquired with a 60,000 resolution was followed by 20 MS/MS spectra, where the most abundant multiply charged ions were selected for MS/MS sequencing with a resolution of 30,000, an automatic gain control target of 5x10⁴ (10H005) or 2x10⁴ (all other samples), an injection time of 100 ms (10H005) or 500 ms (15H023, 15H063, 15H080, 05H149) or 800 ms (all other samples) and collisional energy of 25%.

### Synthetic Peptides

When sufficient amount of material was available, the amino acid sequence of TSA^{hi} was further validated with synthetic peptides, as previously described (Zhao et al., Cancer Immunol Res. 2020 Feb 11 doi: 10.1158/2326-6066.CIR-19-0541. [Epub ahead of print]).

### Bioinformatic Analyses

All analyses were conducted on trimmed data, all alignments were made with star as described in previous section unless otherwise mentioned and all alignments were made on GRCh38.88.

All liquid chromatography (LC)-MS/MS (LC-MS/MS) data were searched against the relevant database using PEAKS X (Bioinformatics Solution Inc.). For peptide identification, tolerance was set at 10 ppm and 0.01 Da for precursor and fragment ions, respectively. The occurrences of oxidation (M) and deamidation (NQ) were set as variable modifications.

1) Identification of MAPs. Following peptide identification, a list of unique peptides was obtained for each sample and a false discovery rate (FDR) of 5% was applied on the peptide scores. Binding affinities to the sample’s HLA alleles were predicted with NetMHC 4.0 (Andreatta and Nielsen, 2016) and only 8 to 11-amino-acid-long peptides with a percentile rank ≤ 2% were used for further annotation.

2) Identification and validation of MAPs of interest (MOI). For both k-mer depletion approaches, this was conducted using a similar approach as described previously (Laumont et al., 2018). Briefly, each MAP and its coding sequence were queried to the relevant AML and normal canonical proteomes (built for all mTECs and MPCs, as detailed above) or cancer and normal 24-nucleotide-long k-mer databases (built from either combined mTECs or combined MPCs, as detailed above), respectively. MAPs detected in the normal canonical proteome were excluded regardless of their coding sequence detection status. MAPs neither detected in the normal canonical proteome nor in normal k-mers were flagged as MOI. MAPs absent from both canonical proteomes but present in both k-mer databases needed to have their RNA coding sequence overexpressed by at least 10-fold in AML compared to normal samples in order to be flagged as MOI. Finally, MAPs corresponding to several RNA sequences (derived from different proteins) could only be flagged as MOI if their respective coding sequences consistently flagged them as MOI.

For the ERE approach, an ERE status of “Yes”, “Maybe” or “No” was given to each individual MAP based on the presence of its amino acid sequence in the ERE and the personalized canonical proteomes. For “Maybe” candidates, the expression levels of the peptide’s coding sequence (i.e. the minimal occurrence of the peptide’s 24-nucleotide-long k-mers set) in the ERE reads and the canonical reads datasets were computed. Only “Maybe” candidates with an expression at least 10-fold higher in the ERE reads dataset were considered as ERE MAPs. Remaining ERE MAP candidates were then manually validated in IGV (Robinson et al., Nat Biotechnol. 2011 Jan;29(1):24-6) to determine if the peptide’s coding sequence contains germline polymorphisms and has an appropriate orientation compared to the ERE sequence and canonical annotated sequences (when applicable).

For the differential k-mer approach, the full list of MAPs was queried to the AML-specific proteome to be flagged as MOI candidate. Next, RNA expression of each MAP (following the procedure described in next section) was evaluated in the 19 AML specimens and in the 11 MPCs used as controls in DE-kupl and flagged as MOI all MAPs having a minimum fold change of 5 between normal and cancer samples. Because the MAP RNA-expression assessment procedure is based on the reference genome to perform its quantifications, candidate MOI deriving from mutations could not be properly quantified and were systematically flagged as MOI candidates. To unambiguously validate the presence at RNA level of each MOI in each AML samples in which they were identified, MOI coding sequences were retrieved from the DiffContigslnfos.tsv output of DE-kupl and queried to the relevant fastq files (sequence in forward R2 fastq and reverse complement in reverse R1 fastq). MOI failing to pass this examination were discarded.

For all lists of MOI candidates (four different approaches), since leucine and isoleucine variants are not distinguishable by standard MS approaches, each list was inspected and MOI for which an existing variant was flagged as non-MOI was discarded unless it presented a higher RNA expression than the variant. MS/MS spectra of all MOI were manually inspected to remove any spurious identifications. Finally, a genomic location was assigned to all MOIs by mapping reads containing their coding sequences on the reference genome using BLAT (tool from the UCSC genome browser). MOI for which reads did not match to a concordant genomic location or which matched to hypervariable regions (such as the MHC, Ig or TCR genes) were excluded. For those with a concordant genomic location, IGV was used to exclude MOI having a coding sequence overlapping a known germline polymorphism (dbSNP149).

3) Quantification of MAP coding sequences in RNA-Seq data. To unambiguously evaluate the RNA expression of each MAP, all MAP amino acid sequences were reverse translated into all possible nucleotide sequences. Next, all these possible sequences were mapped on the genome with GSNAP (Wu et al., 2016), with -n 1000000 option, to locate all genomic regions capable of coding for a given MAP. To confidently capture MAP coded by sequences overlapping splice sites, the possible MAP coding sequences were also mapped on the transcriptome (cDNA & non-coding RNA) to extract (samtools faidx with --length 80 option) large portions (80 nucleotides) of reference transcriptomic sequences that were then mapped on the reference genome (GSNAP, with --use-splicing and --novelsplicing=1 options). For MOI generated by the different TSA discovery pipelines, the genomic alignment of all reads containing their coding sequence was also performed. The outputs of GSNAP were filtered to only keep perfect matches between the sequences and the reference to generate a bed file containing all possible genomic regions susceptible to code for a given MAP. By using samtools view (-F256 option), grep and wc (-I option), the number of reads containing the MAP coding sequences at their respective genomic location in each desired RNA-Seq sample (such as AML, GTEX or normal samples) was counted, aligned on the reference genome with star (bam file). Finally, all read counts (from different regions and coding sequences) for a given MAP were summed and normalized on the total number of reads sequenced in each assessed sample to obtain a reads-per-hundred-million (RPHM) count.

4) Immunogenicity assessments. Immunogenicity predictions of MOI were performed with Repitope (Ogishi and Yotsuyanagi, 2019). Feature computation was performed with the predefined MHCI_Human_MinimumFeatureSet variable and updated (Jul. 12, 2019) FeatureDF_MHCI and FragmentLibrary files provided on the Mendeley repository of the package (https://data.mendeley.com/datasets/sydw5xnxpt/1).

5) MOI presentation and expression by AML patients. To identify all possible HLA alleles capable of presenting a given MAP (promiscuous binders), the MHCcluster online tool (http://www.cbs.dtu.dk/services/MHCcluster/) (Thomsen et al., 2013) was used. HLA alleles having clustering values ≤ 0.4 were considered as capable of presenting the same MAPs. To evaluate the MOI presentation by AML patients in the Leucegene cohort, their HLA type was first determined with Optitype and a given MOI was considered as presented if its expression at RNA level was higher than 2 rphm (instead of 0 rphm, in order to maximize the probability of presentation) and if the patient expressed a HLA allele capable of presenting the MOI (as predicted by NetMHC4.0 for the original identification of the presenting molecule of each discovered MOI and MHCcluster for the identification of promiscuous binders). If a patient expressed two different HLA alleles capable of presenting the same MOI, the MOI was considered as presented twice.

To evaluate the molecular features linked to high TSA^{high} expression, a TSA^{high} was considered as expressed in a given patient if its expression in this patient was higher than its median expression (computed based only on non-null values) across the full cohort. The total number of highly expressed TSAs^{high} (# HE-TSAs^{hi}) was counted for each patient and used to perform correlation analyses with gene expression and association with mutations or other clinical features (see next sections).

6) Survival analyses. Survival data for 374 patients of the Leucegene cohort were a kind gift from the Leucegene team (https://leucegene.ca). Survival analysis was performed to assess the association of high counts of HLA-TSA^{hi} complexes (HLA-restricted presentation of TSAs ^{h}ⁱ), computed as described above, with clinical outcome (overall survival). Patients were separated into two groups depending on the total number of TSAs that they could present: high expressors (upper quartile of HLA-TSAs counts) and low expressors (all the other patients). Survival was compared between the two groups using Kaplan-Meier curves, with significance assessed by log-rank test in GraphPad Prism v7.0. In multivariate analyses, performed with the package survivalAnalysis v0.1.1 in R, age was incorporated as a continuous variable, mutations were coded as present/absent (1/0) and assessment of cytogenetic risk was treated as individual groups and done for intermediate versus favorable risk and adverse versus favorable risk.

7) Mutation analysis. Mutation data for NPM1, FLT3-ITD, FLT3-TKD, IDH1 (R132) and biallelic CEBPA were retrieved from previously published data on the Leucegene cohort (Audemard et al., 2019; Lavallee et al., 2016). Mutations in ASXL1, TP53, DNMT3A, IDH2 (R140 and R172 only), WT1, RUNX1 et TET2 were detected with Freebayes and filtered to remove mutations: (i) having a variant allele frequencies (VAF) < 20%; (ii) flagged as SNPs in the COSMIC database (https://cancer.sanger.ac.uk/cosmic); (iii) having a low putative impact (5’UTR premature start codon gain variant; splice region variant & synonymous variant; stop retained variant; synonymous variant); (iv) missense SNPs having a benign impact on protein structure and function as predicted by FATHMM-XF (http://fathmm.biocompute.org.uk/fathmm-xf/) (Rogers et al., 2018); (iv) insertions and deletions involving AAAAA+ or TTTTT+; (v) mutations flagged only as germline in COSMIC database (Tate et al., 2018).

8) Gene expression analyses. All transcript expression quantifications were performed with kallisto v0.43.0 with default parameters. Kallisto’s transcript-level count estimates were converted into gene-level counts using the R package tximport. EdgeR was used to normalize counts using the TMM algorithm and output count-per-million (cpm) values. Only protein-coding genes (as reported in BioMart tool of Ensembl, useast.ensembl.org/biomart) were retained for further analyses. Systematic Pearson correlations between each gene expression and HE-TSAs^{hi} counts were performed with the cor.test function in R. Correlations were performed on all, non-NPM1/FLT3-ITD/DNMT3A mutated and non-FAB-M1 patients. P-values were corrected for multiple comparisons with the Benjamini & Hochberg method (p.adjust in R) and only genes having a FDR < 0.00001 in at least one of the three correlation analyses, FDR < 0.001 in the three analyses, consistent correlation coefficients (positive or negative) in the three analyses and a correlation coefficient > 0.3 or < -0.3 in at least one analysis were kept for downstream processing.

T-distributed Stochastic Neighbor Embedding (t-SNE) analyses were performed with Rtsne package on the identity of expressed genes obtained from the aggregation of Kallisto’s transcript-level abundance estimates into genes abundance estimates by tximport (expression = 1 if tpm ≥1 and =0 if tpm < 1). Only protein-coding genes (most susceptible to generate MAPs) were used for this analysis.

9) GO term and enrichment map analyses. Biological-process gene-ontology (GO) term over-representation was performed using BiNGO v3.0.3 (Maere et al., 2005) in Cytoscape v3.7.2, using the hypergeometric test and applying a significance cutoff of FDR-adjusted P value of ≤0.005. The output from BiNGO was imported into EnrichmentMap v3.2.1 (Merico et al., 2010) in Cytoscape to cluster redundant GO terms and visualize the results. An EnrichmentMap was generated using a Jaccard similarity coefficient cutoff of 0.25, a P-value cutoff of 0.001 and an FDR-adjusted cutoff of 0.005. The network was visualized using the default “Prefuse Force-Directed Layout” in Cytoscape with default settings and 600 iterations. Groups of similar GO terms were manually circled.

10) Intron retention and NMF clustering. Intron retention (IR) analysis of the full Leucegene cohort and of the 11 main MPC samples has been performed with IRFinder v1.2.5 (Middleton et al., 2017). Introns having IRatio ≥ 10% (introns retained in ≥ 10% of transcripts) and a minimal coverage of 3 reads were considered as retained. Introns were filtered to keep only those retained in at least 2 AML samples and not retained in any MPC sample. The 10% most variable introns (by their coefficient of variation of IRatio across the full cohort) were selected for further analysis (6988 introns). Unsupervised consensus clustering results were generated with NMF v0.21.0 (Gaujoux and Seoighe, 2010) package in R on IRatio of selected introns, with the default Brunet algorithm, and 200 iterations for the rank survey and clustering runs. Cluster result was selected by considering profiles of cophenetic score and average silhouette width of the consensus membership matrix, for clustering solutions having between 3 and 15 clusters.

Abundance heatmap was generated by identifying the top-ranked 2% introns in NMF metagene (W matrix) output file. Removal of duplicate names resulted in a list of 1211 introns. A matrix of these introns IRatios was generated for each Leucegene sample, reordered to match the NMF clustering output, and used the heatmap.3 package in R to perform a hierarchical clustering of introns with a centered correlation distance metric and complete linkage.

### ELISPOT Assays

1) Generation of monocyte-derived dendritic cells. Monocyte-derived dendritic cells were generated from frozen PBMCs, as previously described (Vincent et al., Biology of Blood and Marrow Transplantation: Journal of the American Society for Blood and Marrow Transplantation, 22 Oct. 2013, 20(1):37-45; Laumont et al., Nat Commun. 2016 Jan 5;7:10238). Briefly, DCs were prepared from the adherent PBMC fraction by culture for 8 days in X-VIVO™ 15 medium (Lonza Bioscience) complemented with 5% human serum (Sigma-Aldrich), Sodium pyruvate (1 mM), IL-4 (100 ng/mL, Peprotech) and GM-CSF (100 ng/mL, Peprotech). After 7 days of culture, DCs were matured overnight with IFN-γ (1000 IU/mL, Gibco) and LPS (100 ng/mL, Sigma Aldrich). DCs were loaded with 2 µg/mL of peptide during 2 h after maturation process and were then irradiated (40 Gy) before they were used as APCs in T-DC culture. For the control group, the DCs were pulsed with a mix containing MelanA, NS3 and Gag-A2 peptides (all three binding HLA-A*02:01).

2) In vitro peptide-specific T cell expansion. Thawed PBMCs were first CD8⁺ T-cell enriched using the Human CD8⁺ T cell isolation kit (Miltenyi Biotech) and co-incubated with autologous peptide-pulsed DCs at an APC:T cell ratio of 1: 10. Expanding T cells were cultured for four weeks (with pulsed-DC restimulation every 7 days) in Advanced RPMI medium (Gibco) supplemented with 8% human serum (Sigma-Aldrich), L-glutamine (Gibco) and cytokines. For the first coculture week, IL-12 (10 ng/mL) and IL-21 (30 ng/mL) were added to the medium. Two days after, IL-2 (100 UI/mL) was also added to the cytokine mix. The second week, IL-2 (100 UI/mL), IL-7 (10 ng/mL), IL-15 (5 ng/mL) and IL-21 (30 ng/ml) were added to the medium. For the two last weeks of coculture, IL-2 (100 UI/mL), IL-7 (10 ng/mL) and IL-15 (5 ng/mL) were used. Medium supplemented with the appropriate cytokine mix was added in the cocultures every two days. At the end of the fourth week of coculture, cells were harvested in order to perform an ELISPOT assay.

3) IFNγ ELISPOT assay. ELISpot Human IFNγ (R&D Systems, USA) kit was used according to the manufacturer’s recommendations to perform the experiment. Harvested CD8⁺ T cells were then plated and incubated at 37° C. for 24 hours in the presence of irradiated peptide-pulsed PBMCs (40 Gy) that were used as stimulator cells. As a negative control, sorted CD8⁺ T cells were incubated with irradiated unpulsed PBMCs. Spots were revealed as mentioned in the manufacturer protocol and were counted using an ImmunoSpot S5 UV Analyzer (Cellular Technology Ltd, Shaker Heights, OH). IFN-γ production was expressed as the number of peptide-specific spot-forming cells (SFC) per 10⁶ CD8⁺ T cells after subtracting the spot counts from negative control wells.

### Immunogenicity Predictions

Immunogenicity predictions of MOI were performed with Repitope (Ogishi and Yotsuyanagi, 2019). Feature computation was performed with the predefined MHCI_Human_MinimumFeatureSet variable and updated (Jul. 12, 2019) FeatureDF_MHCI and FragmentLibrary files provided on the Mendeley repository of the package (https://data.mendeley.com/datasets/sydw5xnxpt/1).

### TCR and Cytotoxic T Cell Signature Analyses

TCR repertoire analyses were performed on the RNA-seq data of the 437 Leucegene patients with the TRUST4 software (Li et al., 2017) and default parameters. The clonotype diversity of T cells was estimated by normalizing the number of TCR CDR3s (complete and partial) per kilo TCR reads (CPK). ERGO (Springer et al., 2020) predictions of interactions between complete TCRbeta CDR3 amino acid sequences detected by TRUST4 and MOIs were made through the freely available webportal (http://tcr.cs.biu.ac.il/) with the Autoencoder based model and VDJdb as training databases.

For cytotoxic T cell signature analysis, the count of predicted HLA-TSAs^{hi} pairs per patient was divided by the count of TSAs^{hi} with rphm expressions ≥2 to obtain normalized TSAs^{hi} presentation levels. Patient samples having no HLA-TSAs^{hi} counts or not collected at diagnosis were discarded from the analysis. The remaining 361 patients were grouped according to their normalized TSAs^{hi} presentation levels, and patients above the median of the distribution were compared to the others (below median) through a differential gene expression analysis. This analysis was conducted in R3.6.1. Raw read counts were converted to counts per million (cpm), normalized relative to the library size and lowly expressed genes were filtered out by keeping genes with cpm >1 in at least 2 samples using edgeR 3.26.8 (Robinson et al., 2010) and limma 3.40.6 (Ritchie et al., 2015). This was followed by voom transformations and linear modelling using limma’s Imfit. Finally, moderated t-statistics were computed with eBayes. Genes with p-values ≤ 0.01 and -0.3≥log₂(FC)≥0.3 were considered significantly differentially expressed.

### Cytokine Secretion Assays and Dextramers

Following three rounds of stimulation using peptide loaded monocyte-derived dendritic cells and cytokines based on (Janelle et al., 2015), 1.0 × 10⁶ cells were incubated in presence of 7.5 µg/ml of Brefeldin A (Sigma-Aldrich, Oakville, ON) with either dimethyl sulfoxide (DMSO), 5 µg/ml of the peptide of interest, 5 ug/ml of a control peptide (negative control) or 50 ng/ml of phorbol 12-myristate 13-acetate (PMA) and 500 ng/ml of ionomycin (positive control, Sigma-Aldrich) for 4 hours. Cells were then stained with the cell surface antibodies and fixed and permeabilized using the Cytofix/Cytoperm buffer for intracellular staining according to the manufacturer’s instructions (BD Biosciences, Mississauga, ON). Permeabilized cells were incubated with antibodies directed against IFNγ, IL-2 and TNFα (BD Biosciences) for 20 minutes at 4° C. and resuspended in phosphate buffered saline (PBS) supplemented with 2% fetal bovine serum (FBS; ThermoFisher, Waltham, MA, USA) before acquisition. The acquisition was performed with the LSRII flow cytometer (BD Biosciences) and data were analyzed using FlowJo™ V10 Software (BD Biosciences). For multimer staining, 1.0x10⁶ cells were stained for 45 minutes at 4° C. with custom-made fluorescent dextramers (Immudex, Copenhagen, Denmark) and then stained 30 minutes at 4° C. with CD8 monoclonal antibody (eBiosciences, San Diego, CA). Cells were washed with PBS 2% FBS before acquisition with LSRII cytometer (BD Biosciences). Data were analyzed using FlowJo™ V10 Software (BD Biosciences).

### FEST Assays

For FEST assays, T cells were cultured as previously described, with minor modifications (Danilova et al., 2018). Briefly, on day 0, thawed PBMCs from a healthy donor (BioIVT) were T-cell enriched using the Human Pan T-cell isolation kit (Miltenyi). T cells were resuspended at 2 × 10⁶/mL in AIM V media supplemented with 50 µg/mL gentamicin (ThermoFisher Scientific) and 1% Hepes. The T cell-negative fraction was irradiated at 30 Gy, washed and resuspended at 2.0 × 10⁶/mL in AIM V media supplemented with 50 µg/mL gentamicin and 1% Hepes. One ml per well of both T cells and irradiated T cell-depleted cells were added to a 12-well plate, along with either one of the 3 TSA^{hi} pools (5 TSAs^{hi} per pool, 1 µM final concentration for each TSA) or without peptide. Cells were cultured for 10 days at 37° C., 5% CO2. On day 3 and 7, half the culture media was replaced with fresh culture media containing 100 IU/mL IL-2, 50 ng/mL IL-7, and 50 ng/mL IL-15 (day 3) and 200 IU/mL IL-2, 50 ng/mL IL-7, and 50 ng/mL IL-15 (day 7). On day 10, cells were harvested and CD8⁺ cells were further isolated using the Human CD8⁺ T Cell Isolation Kit (Miltenyi). As a negative control, CD8⁺ T cells were also isolated from freshly thawed uncultured PBMCs of the same healthy donor. DNA was extracted from CD8⁺ T cells using a Qiagen DNA blood mini kit (Qiagen). TCR Vβ CDR3 sequencing was performed using the survey resolution of the ImmunoSEQ platform (Adaptive Biotechnologies). Raw data exported from the immunoSEQ portal were processed with FEST web tool (www.stat-apps.onc.jhmi.edu/FEST) with no minimal number of templates and the “Ignore baseline threshold” parameter.

### Quantification and Statistical Analysis

Unless mentioned clearly in legend of figures, all statistical tests comparing two conditions were made with the Mann-Whitney U test. All correlations were assessed with the Pearson correlation coefficient. Unless mentioned otherwise, all boxes in box plots show the median, 25 and 75th percentiles of the distribution and whiskers extend to the 10^{th} and 90^{th} percentiles. Unless mentioned otherwise, all bar plots show the average with standard deviation (SD). Plots and statistical tests were mainly performed with GraphPad Prism v7.00. For all statistical tests, **** refers to p<0.0001, *** refers to p<0.001, ** refers to p<0.01 and * refers to p<0.05.

### Example 2: Purified Hematopoietic Progenitors Are a Valuable Control for TSA Discovery in AML

MS is the only available technology that can directly identify MAPs (Ehx and Perreault, 2019; Shao et al., 2018). Typically, MS-based identifications of MAPs are performed through the use of software tools matching the acquired tandem MS spectra to a database of protein sequences provided by the user. However, reference protein databases contain only canonical protein sequences and therefore do not allow the identification of MAPs deriving from mutations and aberrantly expressed non-canonical genomic regions (which are the main sources of aeTSAs) (Laumont et al., 2018). A proteogenomic strategy to build MS databases tailored for global TSA identification has been previously described. Customized databases are built for each tumor samples and must meet two criteria: be comprehensive enough to contain all potential TSAs, yet of limited size because inflated reference databases increase the risk of false discoveries (Nesvizhskii et al., 2014; Chong et al., 2020). Database construction begins with (i) RNA-sequencing of the tumor sample, crux of data, (ii) the in silico slicing of RNA-seq reads into 33 nucleotide-long subsequences (k-mers), and (iii) subtraction of normal k-mers in order to create a module containing only cancer-specific k-mers. Like for many aspects of cancer research, the tough question is the selection of the negative control (here, the source of normal k-mers). In previous studies, k-mers from mTECs were used as normal control. However, in the case of AML, another type of negative control was tested: sorted myeloid precursor cells (MPCs, including granulocyte/monocyte progenitors and various types of granulocytic precursors).

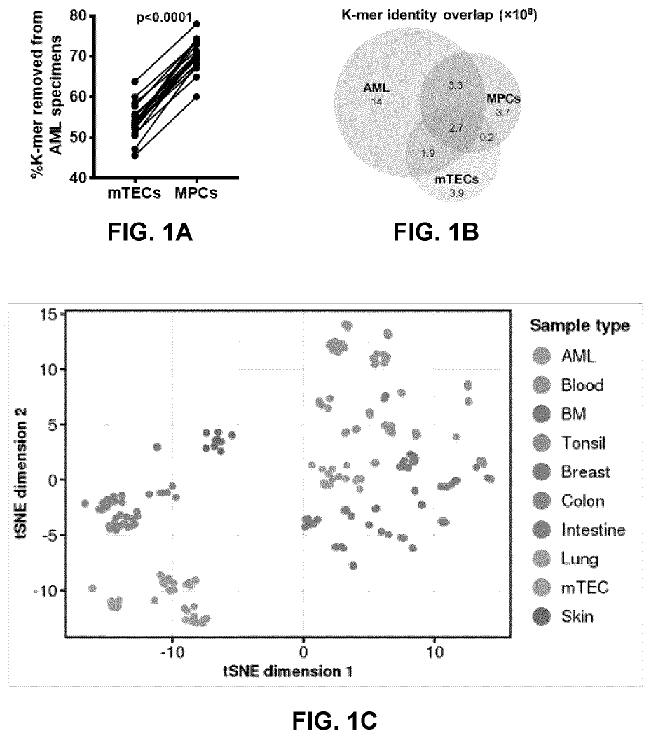

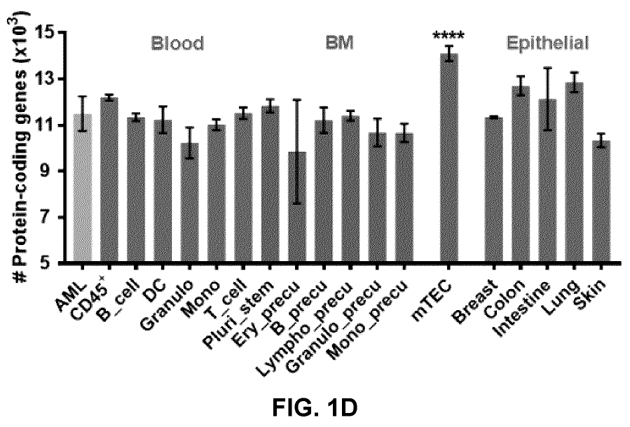

In order to compare the value of mTECs and MPCs as negative controls, the similarity between the 19 target AML specimens (for characteristics, see Table 1), 6 mTEC samples and 6 MPC samples for which high coverage RNA-seq has been performed previously (Maiga et al., 2016) was first compared. Notably, MPCs depleted on average 16.4% more k-mers from AML than mTECs, demonstrating a greater transcriptomic overlap between MPCs and AML than between mTECs and AML (FIG. 1A). Accordingly, and while similar k-mer counts were obtained for both mTECs and MPCs (~8.7 vs ~9.9x10⁸), MPCs shared more exclusive k-mers with AML (~3.3X10⁸, -33%) than mTECs (-1.9x10⁸, ~22%) (FIG. 1B). To establish that the difference of lineage was at the origin of this higher similarity, a t-SNE clustering was performed, based on the identity of expressed protein-coding genes, of the AML samples together with an array of sorted epithelial and hematopoietic cells RNA-seq downloaded from various sources (see methods). This showed that AML samples clustered together with hematopoietic cells while mTECs clustered with epithelial cells (FIG. 1C). Importantly, mTECs expressed the highest diversity of genes, in agreement with their biological function (FIG. 1D). Altogether, these results show that in spite of the diversity of mTECs transcriptomic diversity, MPCs are better normal controls than mTECs for the discovery of TSAs in AML. As a corollary, the size of the AML-specific k-mer database is smaller when MPC k-mers are subtracted instead of mTEC k-mers.

### Example 3: Development of MPC-Based TSA Discovery Approaches

In addition to capture the entire AML TSA landscape, four strategies were evaluated for reference database construction. The first two strategies have been previously reported (FIGS. 2A, B), and the other two are novel (FIGS. 2C, D). Importantly, MS analyses of AML specimens was performed only once and therefore each of the four different TSA-discovery approaches was conducted on the same MS spectra for each AML sample. The first strategy hinges on mTEC subtraction (FIG. 2A) (Laumont et al., 2018). The second focuses specifically on MAPs coded by EREs which could be rich sources of TSAs (FIG. 2B) (Larouche et al., 2020).

In the third strategy, k-mers from both mTECs and MPCs were depleted (FIG. 2C). Of note, depletion steps are preceded by a filtering of k-mers based on their occurrence (the number of times that a k-mer is present in the same sample, FIG. 8A, B) in order to limit the final number of k-mers to ~30 million for contig assembly (the assembly of more k-mers being too demanding in terms of computation time). As a result, depletion of mTECs+MPCs k-mers removed more k-mers from AML samples than mTECs only, decreasing ~2-3 times the occurrence thresholds and thereby enabling the discovery of MAPs missed with the mTECs k-mer depletion approach databases (FIGS. 8C, D).

The fourth strategy was aimed at circumventing the main caveat of the k-mer depletion strategy: the absence of comparisons between k-mer abundance in normal and cancer samples. Specifically, in k-mer depletion strategies, the presence of a given k-mer, even with an occurrence of one, in normal controls results in the filtering of this k-mer in cancer samples, even if its frequency is 100-fold higher in cancer relative to normal controls. Briefly, differential k-mer expression (DKE) analysis was performed using the DE-kupl computational protocol (Audoux et al., 2017), with some in-house adjustments, and can be summarized as follows (FIG. 2D and FIG. 9A): (i) pre-filtering of k-mers being present (occurrence ≥3) in at least 30% of AML samples; (ii) normalization of k-mer abundance; (iii) statistical comparison of k-mer abundance through a user-defined algorithm; (iv) assembly of significantly differentially-overexpressed k-mers (minimum fold change of 10) into contigs and (v) alignment of contigs on genome to establish their region of origin. Because they were the most closely-related normal samples, MPCs were selected and used as normal controls in the present study, and the 19 AML samples were compared to the 11 available high-coverage MPC samples. Next, personalized contig sequences were generated for each AML samples (based on read coverage and SNP calling in the genomic positions of the differentially expressed contigs), translated into all possible reading frames and combined with a personalized canonical proteome to perform MAPs identifications (FIG. 2).

### Example 4: MPCs-Based Approaches Identify the Majority of TSA^{hi} in AML

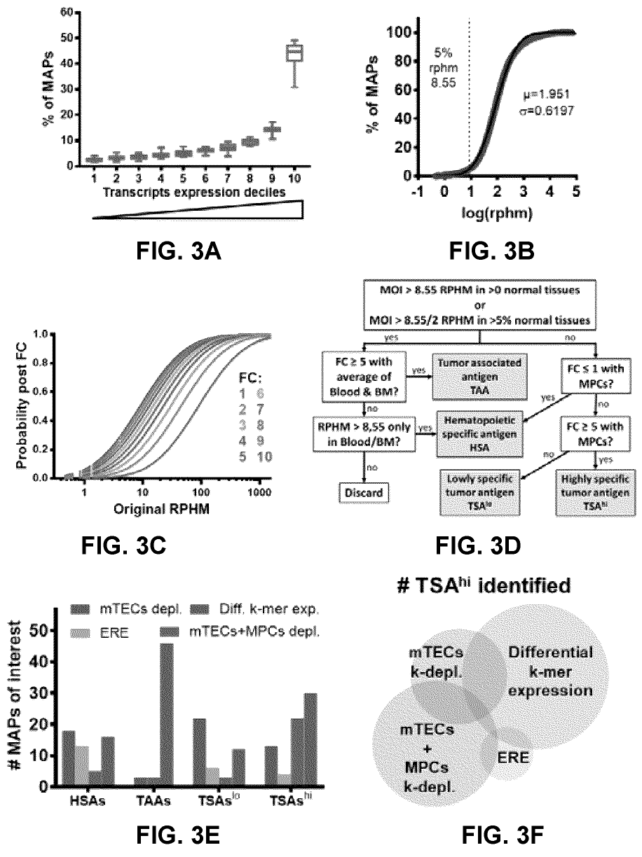

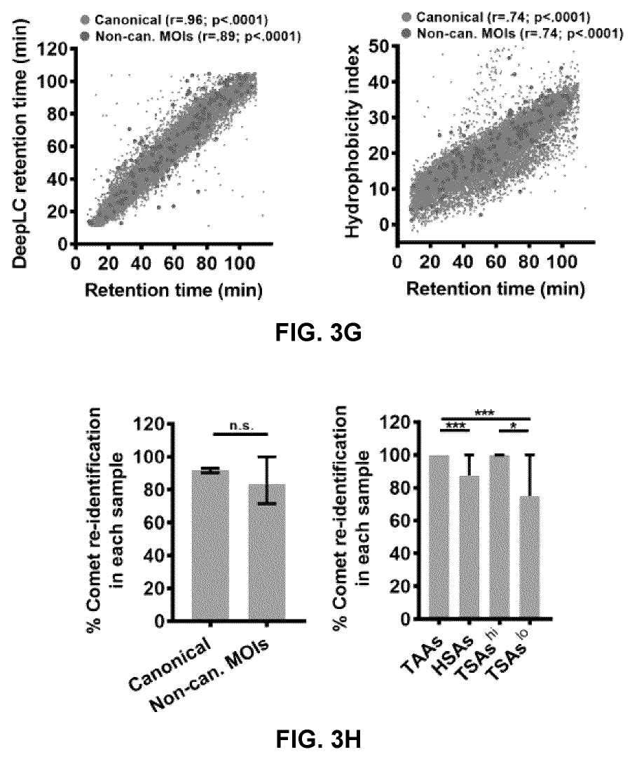

Each of the four TSA-discovery approaches identified thousands of MAPs across the 19 AML samples (Table 2). To be considered an actionable TSA, a MAP would need to be presented abundantly by AML cells and be either not presented by normal cells or presented at levels low enough not to trigger T-cell recognition, as epitope density plays a key role eradication of target cells by CD8 T cells (Cosma and Eisenlohr, 2019). Because MAPs preferentially derive from highly abundant transcripts (FIG. 3A and Pearson et al., 2016), two important thresholds were established: (i) an RNA expression level below which the probability of generating a MAP in normal tissues can be considered as low and (ii) the RNA expression fold change (FC) necessary to increase drastically the probability of presenting a MAP. To achieve this, the RNA expression of all identified MAPs in their respective AML sample was evaluated, and it was found that it obeyed to a normal distribution that was plotted as cumulative frequency distribution (FIG. 3B). This evidenced that with an expression below 8.55 reads per hundred million (RPHM), the probability to generate a MAP was <5%. Given that AML cells express similar levels of MHC molecules compared to normal granulocytes and that granulocytes express the highest levels of MHC-I among normal tissues (Berlin et al., 2015; Boegel et al., 2018), 8.55 RPHM was established as a first threshold for all tissues. Based on the same distribution, the impact of different FCs on the probability to generate a MAP could also be evaluated (FIG. 3C). This showed that FCs from 2 to 5 tended to have higher impacts on probabilities than greater FCs. Five (5) was therefore adopted as minimum FC threshold.

Based on these two thresholds, a decision tree was established to segregate MAPs based on their RNA expression in AML, MPCs, other normal hematopoietic cells and a wide range of normal adult tissues, including mTECs (FIG. 3D). In brief, all MAPs being expressed below 8.55 RPHM in normal tissues and being expressed at higher levels in AML than in MPCs were flagged as TSAs because their detection is an evidence of their presentation at AML cells surface while they have low probabilities of being presented by normal tissues. In addition, TSAs with FC of at least 5 between AML and MPCs were flagged as TSA^{hi} because they had the highest probability of being exclusively presented by AML cells. Other MAPs overexpressed in hematopoietic cells relative to other tissues but failing to meet these criteria were classified as TAAs or hematopoietic specific antigens (HSAs) (FIG. 3D).

After the prefiltering steps of each pipeline (see methods) and categorization based on the decision tree, four lists of MAPs of interest (MOI) were obtained (Table 2). The mTECs depletion approach yielded the highest proportion of HSAs while the vast majority of TSAs^{hi} were identified by MPC-based approaches (FIGS. 3E, F and 10A). Because the DKE approach prefilters k-mers having a minimal occurrence in a minimum of patients, the overlap between both MPCs-based approaches was low. Accordingly, most TSAs^{hi} identified by the depletion approach presented a low inter-patient sharing compared to those identified by the DKE approach (FIG. 9B). Altogether, these results show that the DKE approach is the best suited to identify TSAs^{hi} in AML, and that it can be complemented by the MPCs-based k-mer depletion approach to identify additional less-shared TSAs^{hi}.

TABLE 2

Details of MOIs identified through the four proteogenomic approaches.

| MOI Sequence (SEQ ID NO:) | Classific. | Method of identification | Biotype | Gene (if exonic or intronic) |
|---|---|---|---|---|
| RQISVQASL (1) | HSA | Differential k-mer expression | Intron | PPP6R2 |
| DRELRNLEL (2) | HSA | Differential k-mer expression | Coding exon | PYHIN1 |
| GARQQIHSW (3) | HSA | Differential k-mer expression | Coding exon | FADS1 |
| SGKLRVAL (4) | HSA | Differential k-mer expression | Coding exon | TBX21 |
| RSASSATQVHK (5) | HSA | Differential k-mer expression | Intergenic | |
| SASSATQVHK (6) | HSA | Differential k-mer expression | Intergenic | |
| FLLEFKPVS (7) | HSA | Differential k-mer expression | Intron | CEP83 |
| GPQVRGSI (8) | HSA | Differential k-mer expression | Intron | UBE3C |
| IRMKAQAL (9) | HSA | Differential k-mer expression | Intron | KMT2C |
| KIKVFSKVY (10) | HSA | Differential k-mer expression | Intron | TRPM7 |
| LLSRGLLFRI (11) | HSA | Differential k-mer expression | Intron | ANKIB1 |
| LPIASASLL (12) | HSA | Differential k-mer expression | Intron | MAML3 |
| LYFLGHGSI (13) | HSA | Differential k-mer expression | Intron | KMT2C |
| NPLQLSLSI (14) | HSA | Differential k-mer expression | Intron | ARL15 |
| DLMLRESL (15) | HSA | Differential k-mer expression | ncRNA | RP11-477N3.1 |
| VTFKLSLF (16) | HSA | Differential k-mer expression & ERE | ERE | |
| IALYKQVL (17) | HSA | ERE | ERE | |
| IVATGSLLK (18) | HSA | ERE | ERE | |
| KIKNKTKNK (19) | HSA | ERE | ERE | |
| KLLSLTIYK (20) | HSA | ERE | ERE | |
| NILKKTVL (21) | HSA | ERE | ERE | |
| NPKLKDIL (22) | HSA | ERE | ERE | |
| NQKKVRIL (23) | HSA | ERE | ERE | |
| PFPLVQVEPV (24) | HSA | ERE | ERE | |
| SPQSGPAL (25) | HSA | ERE | ERE | |
| TSRLPKIQK (26) | HSA | ERE | ERE | |
| LLDNILQSI (27) | HSA | ERE & mTECs k-mer depletion | ERE | |
| RLEVRKVIL (28) | HSA | mTECs k-mer depletion | Exon-Intron | DOCK8 |
| LSWGYFLFK (29) | HSA | mTECs k-mer depletion | Intergenic | |
| TILPRILTL (30) | HSA | mTECs k-mer depletion | Intergenic | |
| EGKIKRNI (31) | HSA | mTECs k-mer depletion | Intron | SLC25A13 |
| FLASFVEKTVL (32) | HSA | mTECs k-mer depletion | Intron | OS9 |
| ILASHNLTV (33) | HSA | mTECs k-mer depletion | Intron | RPS6KC1 |
| IQLTSVHLL (34) | HSA | mTECs k-mer depletion | Intron | PTPRC |
| LELlSFLPVL (35) | HSA | mTECs k-mer depletion | Intron | CDCA8 |
| NFCMLHQSI (36) | HSA | mTECs k-mer depletion | Intron | IKZF1 |
| PARPAGPL (37) | HSA | mTECs k-mer depletion | Intron | INPP5D |
| PLPIVPAL (38) | HSA | mTECs k-mer depletion | Intron | USP3 |
| SNLIRTGSH (39) | HSA | mTECs k-mer depletion | Intron | INPP5D |
| VPAPAQAI (40) | HSA | mTECs k-mer depletion | Intron | PAN3 |
| KGHGGPRSW (41) | HSA | mTECs k-mer depletion | ncRNA | AC104232.1 |
| ITSSAVTTALK (42) | HSA | mTECs k-mer depletion | UTR | LMO2 |
| LLLPESPSI (43) | HSA | mTECs+MPCs k-mer depletion & ERE & mTECs k-mer depletion | ERE | |
| VILIPLPPK (44) | HSA | mTECs+MPCs k-mer depletion & mTECs k-mer depletion | ERE antisense | |
| AVLLPKPPK (45) | HSA | mTECs+MPCs k-mer depletion | ERE Antisense | |
| TQVSMAESI (46) | HSA | mTECs+MPCs k-mer depletion | ERE | |
| LNHLRTSI (47) | HSA | mTECs+MPCs k-mer depletion | Intron | ZNF407 |
| NTSHLPLIY (48) | TAA | Differential k-mer expression | Intron | COL4A5 |
| SIQRNLSL (49) | TAA | Differential k-mer expression | Coding exon | PIEZO2 |
| NVSSHVHTV (50) | TAA | Differential k-mer expression | Coding exon | TPSB2 |
| ALASHLIEA (51) | TAA | Differential k-mer expression | Coding exon | EHD2 |
| ALDDITIQL (52) | TAA | Differential k-mer expression | Coding exon | MAMDC2 |
| ALGNTVPAV (53) | TAA | Differential k-mer expression | Coding exon | DNAAF3 |
| ALLPAVPSL (54) | TAA | Differential k-mer expression | Coding exon | WT1 |
| APAPPPVAV (55) | TAA | Differential k-mer expression | Coding exon | IRX3 |
| APDKKITL (56) | TAA | Differential k-mer expression | Coding exon | FOXC1 |
| AQMNLLQKY (57) | TAA | Differential k-mer expression | Coding exon | DLC1 |
| DQVIRLAGL (58) | TAA | Differential k-mer expression | Coding exon | PTPN14 |
| ETTSQVRKY (59) | TAA | Differential k-mer expression | Coding exon | MMRN1 |
| GGSLIHPQW (60) | TAA | Differential k-mer expression | Coding exon | TPSB2 |
| GLYYKLHNV (61) | TAA | Differential k-mer expression | Coding exon | GATA2 |
| GQKPVILTY (62) | TAA | Differential k-mer expression | Coding exon | IGSF10 |
| GSLDFQRGW (63) | TAA | Differential k-mer expression | Coding exon | ANGPT1 |
| HHLVETLKF (64) | TAA | Differential k-mer expression | Coding exon | GGT5 |
| HLLSETPQL (65) | TAA | Differential k-mer expression | Coding exon | IGSF10 |
| HQLYRASAL (66) | TAA | Differential k-mer expression | Coding exon | TTC28 |
| HTDDIENAKY (67) | TAA | Differential k-mer expression | Coding exon | PTPN14 |
| IAAPILHV (68) | TAA | Differential k-mer expression | Coding exon | GGT5 |
| KAFPFHIIF (69) | TAA | Differential k-mer expression | Coding exon | GUCY1B3 |
| KATEYVHSL (70) | TAA | Differential k-mer expression | Coding exon | MYCN |
| KFSNVTMLF (71) | TAA | Differential k-mer expression | Coding exon | GUCY1A3 |
| KLLEKAFSI (72) | TAA | Differential k-mer expression | Coding exon | CYP7B1 |
| KPMPTKVVF (73) | TAA | Differential k-mer expression | Coding exon | MAMDC2 |
| NVNRPLTMK (74) | TAA | Differential k-mer expression | Coding exon | GATA2 |
| REPYELTVPAL (75) | TAA | Differential k-mer expression | Coding exon | RBPMS |
| SEAEAAKNAL (76) | TAA | Differential k-mer expression | Coding exon | RBPMS |
| SLWGQPAEA (77) | TAA | Differential k-mer expression | Coding exon | COL4A5 |
| SPADHRGYASL (78) | TAA | Differential k-mer expression | Coding exon | SOX4 |
| SPQSAAAEL (79) | TAA | Differential k-mer expression | Coding exon | FOXC1 |
| SPVVHQSL (80) | TAA | Differential k-mer expression | Coding exon | PTPN14 |
| SPYRTPVL (81) | TAA | Differential k-mer expression | Coding exon | IGSF10 |
| SVFAGVVGV (82) | TAA | Differential k-mer expression | Coding exon | GUCY1A3 |
| SYSPAHARL (83) | TAA | Differential k-mer expression | Coding exon | GATA2 |
| THGSEQLHL (84) | TAA | Differential k-mer expression | Coding exon | IGSF10 |
| TQAPPNVVL (85) | TAA | Differential k-mer expression | Coding exon | DENND6B |
| VLVPYEPPQV (86) | TAA | Differential k-mer expression | Coding exon | TP63 |
| VSFPDVRKV (87) | TAA | Differential k-mer expression | Coding exon | NEGR1 |
| VVFDKSDLAKY (88) | TAA | Differential k-mer expression | Coding exon | SLC45A3 |
| YSHHSGLEY (89) | TAA | Differential k-mer expression | Coding exon | CAV2 |
| YYLDWIHHY (90) | TAA | Differential k-mer expression | Coding exon | TPSB2 |
| SVYKYLKAK (91) | TAA | Differential k-mer expression | UTR | IRX3 |
| IYQFIMDRF (92) | TAA | Differential k-mer expression | Coding exon | FOXC1 |
| GTLQGIRAW (93) | TAA | Differential k-mer expression & mTECs+MPCs k-mer depletion | Coding exon | NKX2-3 |
| AQKVSVGQAA (94) | TAA | mTECs k-mer depletion | UTR | LMO2 |
| LYPSKLTHF (95) | TAA | mTECs+MPCs k-mer depletion & mTECs k-mer depletion | Intergenic | |
| ATQNTIIGK (96) | TAA | mTECs+MPCs k-mer depletion & mTECs k-mer depletion | Out-of-frame translation | CPA3 |
| AQDIILQAV (97) | TSA^{hi} | Differential k-mer expression | Intergenic | |
| PPRPLGAQV (98) | TSA^{hi} | Differential k-mer expression | UTR | C16orf87 |
| FNVALNARY (99) | TSA^{hi} | Differential k-mer expression | Coding exon | LTBP1 |
| GPGSRESTL (100) | TSA^{hi} | Differential k-mer expression | Coding exon | PLPPR3 |
| IPHQRSSL (101) | TSA^{hi} | Differential k-mer expression | Coding exon | GCSAML |
| LTDRIYLTL (102) | TSA^{hi} | Differential k-mer expression | Coding exon | DNAH10 |
| NLKEKKALF (103) | TSA^{hi} | Differential k-mer expression | Coding exon | ST8SIA6 |
| VLFGGKVSGA (104) | TSA^{hi} | Differential k-mer expression | Coding exon | MFSD2B |
| VVFPFPVNK (105) | TSA^{hi} | Differential k-mer expression | Coding exon | MYCN |
| SLLIIPKKK (106) | TSA^{hi} | Differential k-mer expression | ERE | |
| APGAAGQRL (107) | TSA^{hi} | Differential k-mer expression | Intergenic | |
| KLQDKEIGL (108) | TSA^{hi} | Differential k-mer expression | Intergenic | |
| SLREPQPAL (109) | TSA^{hi} | Differential k-mer expression | Intergenic | |
| TPGRSTQAI (110) | TSA^{hi} | Differential k-mer expression | Intergenic | |
| APRGTAAL (111) | TSA^{hi} | Differential k-mer expression | Intron | JADE1 |
| IASPIALL (112) | TSA^{hi} | Differential k-mer expression | Intron | ELL |
| ILFQNSALK (113) | TSA^{hi} | Differential k-mer expression | Intron | SLC25A25 |
| ILKKNISI (114) | TSA^{hi} | Differential k-mer expression | Intron | AKAP13 |
| IPLAVRTI (115) | TSA^{hi} | Differential k-mer expression | Intron | FBXO28 |
| LPRNKPLL (116) | TSA^{hi} | Differential k-mer expression | Intron | IGF2BP2 |
| PAPPHPAAL (117) | TSA^{hi} | Differential k-mer expression | Intron | HOOK2 |
| SPVVRVGL (118) | TSA^{hi} | Differential k-mer expression | Intron | DNAH7 |
| TLNQGINVYI (119) | TSA^{hi} | Differential k-mer expression | Intron | SLC39A10 |
| RPRGPRTAP (120) | TSA^{hi} | Differential k-mer expression | UTR | PIEZO1 |
| SVQLLEQAIHK (121) | TSA^{hi} | Differential k-mer expression & ERE | ERE | |
| RTPKNYQHW (122) | TSA^{hi} | Differential k-mer expression & mTECs k-mer depletion | Coding exon | LINC01835 |
| ALPVALPSL (123) | TSA^{hi} | Differential k-mer expression & mTECs k-mer depletion | Intron | RFX2 |
| SLQILVSSL (124) | TSA^{hi} | Differential k-mer expression & mTECs k-mer depletion | Intron | LRRC8C |
| ISNKVPKLF (125) | TSA^{hi} | Differential k-mer expression & mTECs k-mer depletion | ncRNA | LINC02147 |
| TVIRIAIVNK (126) | TSA^{hi} | Differential k-mer expression & mTECs+MPCs k-mer depletion & mTECs k-mer depletion | Intron | FTO |
| KEIFLELRL (127) | TSA^{hi} | ERE | ERE | |
| TLRSPGSSL (128) | TSA^{hi} | ERE | ERE | |
| TVRGDVSSL (129) | TSA^{hi} | mTECs k-mer depletion | Intergenic | |
| ALDPLLLRI (130) | TSA^{hi} | mTECs k-mer depletion | Intron | CETP |
| ISLIVTGLK (131) | TSA^{hi} | mTECs k-mer depletion | Intron | ASPH |
| KILDVNLRI (132) | TSA^{hi} | mTECs k-mer depletion | Intron | SIK3 |
| ERVYIRASL (133) | TSA^{hi} | mTECs k-mer depletion | ncRNA | AL359636.1 |
| ILDLESRY (134) | TSA^{hi} | mTECs+MPCs k-mer depletion | ERE | |
| KTFVQQKTL (135) | TSA^{hi} | mTECs+MPCs k-mer depletion | ERE | |
| LYIKSLPAL (136) | TSA^{hi} | mTECs+MPCs k-mer depletion | ERE | |
| VLKEKNASL (137) | TSA^{hi} | mTECs+MPCs k-mer depletion | ERE | |
| LGISLTLKY (138) | TSA^{hi} | mTECs+MPCs k-mer depletion | Intergenic | |
| DLLPKKLL (139) | TSA^{hi} | mTECs+MPCs k-mer depletion | Intron | BACH1 |
| HSLISIVYL (140) | TSA^{hi} | mTECs+MPCs k-mer depletion | Intron | XACT |
| IAGALRSVL (141) | TSA^{hi} | mTECs+MPCs k-mer depletion | Intron | TNS3 |
| IGNPILRVL (142) | TSA^{hi} | mTECs+MPCs k-mer depletion | Intron | EFR3A |
| IYAPHIRLS (143) | TSA^{hi} | mTECs+MPCs k-mer depletion | Intron | ERC1 |
| LRSQILSY (144) | TSA^{hi} | mTECs+MPCs k-mer depletion | Intron | LINC00484 |
| RYLANKIHI (145) | TSA^{hi} | mTECs+MPCs k-mer depletion | Intron | ARHGAP32 |
| SLLSGLLRA (146) | TSA^{hi} | mTECs+MPCs k-mer depletion | Intron | CD34 |
| SRIHLVVL (147) | TSA^{hi} | mTECs+MPCs k-mer depletion | Intron | ZNF280C |
| SSSPVRGPSV (148) | TSA^{hi} | mTECs+MPCs k-mer depletion | Intron | PTK2 |
| STFSLYLKK (149) | TSA^{hi} | mTECs+MPCs k-mer depletion | Intron | DTNA |
| SLDLLPLSI (150) | TSA^{hi} | mTECs+MPCs k-mer depletion | ncRNA | LINC00996 |
| VTDLLALTV (151) | TSA^{hi} | mTECs+MPCs k-mer depletion & ERE & mTECs k-mer depletion | ERE | |
| RTQITKVSLKK (152) | TSA^{hi} | mTECs+MPCs k-mer depletion & mTECs k-mer depletion | Intergenic | |
| ILRSPLKW (153) | TSA^{hi} | mTECs+MPCs k-mer depletion & mTECs k-mer depletion | Intron | PIEZO2 |
| LSTGHLSTV (154) | TSA^{hi} | mTECs+MPCs k-mer depletion & mTECs k-mer depletion | Intron | MYO16 |
| TVEEYLVNI (155) | TSA^{lo} | Differential k-mer expression | Intron | ECSIT |
| QIKTKLLGSL (156) | TSA^{lo} | Differential k-mer expression | Intron | KIF27 |
| LPSFSHFLLL (157) | TSA^{lo} | Differential k-mer expression | Intergenic | |
| CLRIGPVTL (158) | TSA^{lo} | Differential k-mer expression | Intron | ZDHHC14 |
| HVSDGSTALK (159) | TSA^{lo} | Differential k-mer expression | Intron | PDE4D |
| IAYSVRALR (160) | TSA^{lo} | Differential k-mer expression | Intron | ACBD3 |
| PRGFLSAL (161) | TSA^{lo} | Differential k-mer expression | Intron | ZNF26 |
| ISSWLISSL (162) | TSA^{lo} | Differential k-mer expression & ERE | ERE | |
| IPLNPFSSL (163) | TSA^{lo} | Differential k-mer expression & mTECs k-mer depletion | ERE | |
| LSDRQLSL (164) | TSA^{lo} | Differential k-mer expression & mTECs k-mer depletion | Intron | CDK6 |
| LSHPAPSSL (165) | TSA^{lo} | Differential k-mer expression & mTECs k-mer depletion | Intron | RNF220 |
| LRKAVDPIL (166) | TSA^{lo} | Differential k-mer expression & mTECs+MPCs k-mer depletion | Intron | SLC39A11 |
| ILLEEQSLI (167) | TSA^{lo} | ERE | ERE | |
| LTSISIRPV (168) | TSA^{lo} | ERE | ERE | |
| TISECPLLI (169) | TSA^{lo} | ERE | ERE | |
| TLKLKKIFF (170) | TSA^{lo} | ERE | ERE | |
| ILLSNFSSL (171) | TSA^{lo} | mTECs k-mer depletion | ERE | |
| LGGAWKAVF (172) | TSA^{lo} | mTECs k-mer depletion | ERE | |
| LSASHLSSL (173) | TSA^{lo} | mTECs k-mer depletion | ERE | |
| AGDIIARLI (174) | TSA^{lo} | mTECs k-mer depletion | Intron | ZWILCH |
| DRGILRNLL (175) | TSA^{lo} | mTECs k-mer depletion | Intron | ABI1 |
| GLRLIHVSL (176) | TSA^{lo} | mTECs k-mer depletion | Intron | KLF13 |
| GLRLLHVSL (177) | TSA^{lo} | mTECs k-mer depletion | Intron | VAV3 |
| LHNEKGLSL (178) | TSA^{lo} | mTECs k-mer depletion | Intron | ZNF804A |
| LPSFSRPSGII (179) | TSA^{lo} | mTECs k-mer depletion | Intron | CCDC26 |
| LSSRLPLGK (180) | TSA^{lo} | mTECs k-mer depletion | Intron | ZNF367 |
| MIGIKRLL (181) | TSA^{lo} | mTECs k-mer depletion | Intron | TRIP12 |
| NLKKREIL (182) | TSA^{lo} | mTECs k-mer depletion | Intron | POGLUT1 |
| RMVAYLQQL (183) | TSA^{lo} | mTECs k-mer depletion | Intron | PTEN |
| SPARALPSL (184) | TSA^{lo} | mTECs k-mer depletion | Intron | RFX8 |
| TVPGIQRY (185) | TSA^{lo} | mTECs k-mer depletion | Intron | ERBIN |
| VSRNYVLLI (186) | TSA^{lo} | mTECs k-mer depletion | Intron | SLC39A10 |
| LTVPLSVFW (187) | TSA^{lo} | mTECs k-mer depletion | Pseudogene | SMURF2P1-LRRC37BP1 |
| KLNQAFLVL (188) | TSA^{lo} | mTECs k-mer depletion | Intron | SNTB1 |
| RLVSSTLLQK (189) | TSA^{lo} | mTECs+MPCs k-mer depletion & ERE & mTECs k-mer depletion | ncRNA | AC128707.1 |
| LPSHSLLI (190) | TSA^{lo} | mTECs+MPCs k-mer depletion | Intron | MAPRE2 |

| MOI | Immunogenicity score | AML/MPC | Identified in samples | HLA |
|---|---|---|---|---|
| RQISVQASL (1) | 0.3896 | 0.143096844 | 15H080 | HLA-B*27:05 |
| DRELRNLEL (2) | 0.36072 | 106.6504772 | 08H039 | HLA-B*14:02 |
| GARQQIHSW (3) | 0.31568 | 8.852871755 | 07H122 | HLA-B*57:01 |
| SGKLRVAL (4) | 0.38024 | 277.184 | 05H143, 07H060 | HLA-B*08:01 |
| RSASSATQVHK (5) | 0.27736 | 21.15087756 | 15H023 | HLA-A*03:01 |
| SASSATQVHK (6) | 0.33728 | 22.04593113 | 08H053 | HLA-A*11:01 |
| FLLEFKPVS (7) | 0.32488 | 0.178033826 | 14H124 | HLA-A*02:01 |
| GPQVRGSI (8) | 0.43896 | 0.244436854 | 07H063 | HLA-B*07:02 |
| IRMKAQAL (9) | 0.30384 | 0.14128544 | 07H063 | HLA-C*06:02 |
| KIKVFSKVY (10) | 0.15168 | 0.563808415 | 12H172 | HLA-B*15:01 |
| LLSRGLLFRI (11) | 0.40744 | 0.337716083 | 14H124 | HLA-A*02:01 |
| LPIASASLL (12) | 0.50848 | 0.941520982 | 15H023 | HLA-B*51:01 |
| LYFLGHGSI (13) | 0.3268 | 0.254931815 | 16H123 | HLA-A*24:02 |
| NPLQLSLSI (14) | 0.45888 | 0.4888543537 | 07H060 | HLA-B*08:01 |
| DLMLRESL (15) | 0.4704 | 0.543741144 | 07H060 | HLA-B*08:01 |
| VTFKLSLF (16) | 0.38384 | 0.890019017 | 05H143 | HLA-B*57:01 |
| IALYKQVL (17) | 0.37872 | 0.841619533 | 05H143 | HLA-B*08:01 |
| IVATGSLLK (18) | 0.406 | 0.506704612 | 15H023 | HLA-A*03:01 |
| KIKNKTKNK (19) | 0.386 | 0.846822574 | 16H123 | HLA-A*03:01 |
| KLLSLTIYK (20) | 0.2392 | 0.360396852 | 16H123 | HLA-A*03:01 |
| NILKKTVL (21) | 0.42712 | 0.995471668 | 15H063 | HLA-B*08:01 |
| NPKLKDIL (22) | 0.37752 | 1.97918996 | 05H143 | HLA-B*08:01 |
| NQKKVRIL (23) | 0.33296 | 0.456777709 | 07H060 | HLA-B*08:01 |
| PFPLVQVEPV (24) | 0.48656 | 0.406411352 | 15H023 | HLA-B*51:01 |
| SPQSGPAL (25) | 0.44536 | 0.540664564 | 16H123 | HLA-B*07:02 |
| TSRLPKIQK (26) | 0.29104 | 0.536699874 | 07H063 | HLA-A*30:01 |
| LLDNILQSI (27) | 0.42688 | 2.344739646 | 15H023 | HLA-A*02:01 |
| RLEVRKVIL (28) | 0.2716 | 0.619722353 | 15H063 | HLA-B*08:01 |
| LSWGYFLFK (29) | 0.27096 | 2.09747864 | 07H063 | HLA-A*30:01 |
| TILPRILTL (30) | 0.4464 | 0.603930045 | 16H123 | HLA-C*07:02 |
| EGKIKRNI (31) | 0.36296 | 0.79453462 | 15H063 | HLA-B*08:01 |
| FLASFVEKTVl (32) | 0.2804 | 0.317704238 | 14H124 | HLA-A*02:01 |
| ILASHNLTV (33) | 0.35944 | 0.836592866 | 16H145 | HLA-A*02:01 |
| IQLTSVHLL (34) | 0.37928 | 0.364630795 | 15H080 | HLA-A*02:01 |
| LELISFLPVL (35) | 0.42824 | 0.089927659 | 14H124 | HLA-A*02:01 |
| NFCMLHQSI (36) | 0.42648 | 0.548351918 | 12H172 | HLA-A*24:02 |
| PARPAGPL (37) | 0.44312 | 0.830983729 | 16H145 | HLA-C*03:03 |
| PLPIVPAL (38) | 0.51424 | 0.60208428 | 15H023 | HLA-B*51:01 |
| SNLIRTGSH (39) | 0.29392 | 0.834424495 | 08H039 | HLA-B*14:02 |
| VPAPAQAI (40) | 0.462 | 0.085637644 | 16H123 | HLA-B*07:02 |
| KGHGGPRSW (41) | 0.25872 | 0.352735092 | 07H122 | HLA-B*57:01 |
| ITSSAVTTALK (42) | 0.37744 | 2.017525959 | 11H008, 15H023, 16H123 | HLA-A*03:01 |
| LLLPESPSI (43) | 0.39184 | 0.178058578 | 08H039 | HLA-A*02:01 |
| VILIPLPPK (44) | 0.40664 | 0.262280081 | 11H035 | HLA-A*03:01 |
| AVLLPKPPK (45) | 0.35024 | 0.545425585 | 10H005 | HLA-A*11:01 |
| TQVSMAESI (46) | 0.34456 | 0.759898781 | 07H141 | HLA-B*38:01 |
| LNHLRTSI (47) | 0.4288 | 0.275551012 | 05H143 | HLA-B*08:01 |
| NTSHLPLIY (48) | 0.24416 | 405.694 | 07H060 | HLA-A*01:01 |
| SIQRNLSL (49) | 0.32568 | 19.18764464 | 07H060 | HLA-B*08:01 |
| NVSSHVHTV (50) | 0.27864 | 7.343047581 | 05H149 | HLA-A*68:02 |
| ALASHLIEA (51) | 0.07824 | 15.39591183 | 08H039, 15H063 | HLA-A*02:01 |
| ALDDITIQL (52) | 0.36416 | 17.31517663 | 05H143 | HLA-A*02:01 |
| ALGNTVPAV (53) | 0.4856 | 14.04712651 | 07H122 | HLA-A*02:01 |
| ALLPAVPSL (54) | 0.47288 | 1261.102 | 05H143, 07H122, 16H145 | HLA-A*02:01 |
| APAPPPVAV (55) | 0.53488 | 344.1774537 | 16H123 | HLA-B*07:02 |
| APDKKITL (56) | 0.2792 | 55.02751683 | 11H035 | HLA-B*07:02 |
| AQMNLLQKY (57) | 0.26056 | 31.38609992 | 12H172 | HLA-B*15:01 |
| DQVIRLAGL (58) | 0.44592 | 246.7135986 | 08H039 | HLA-B*14:02 |
| ETTSQVRKY (59) | 0.15824 | 9.473637252 | 07H141, 14H124 | HLA-A*26:01 |
| GGSLIHPQW (60) | 0.45512 | 11.49478271 | 15H013 | HLA-B*57:03 |
| GLYYKLHNV (61) | 0.23424 | 13.52738214 | 07H122 | HLA-A*02:01 |
| GQKPVILTY (62) | 0.21208 | 5.212085157 | 16H123 | HLA-B*15:01 |
| GSLDFQRGW (63) | 0.31896 | 19.21014006 | 07H122 | HLA-B*57:01 |
| HHLVETLKF (64) | 0.23928 | 66.82447284 | 07H141 | HLA-B*38:01 |
| HLLSETPQL (65) | 0.51952 | 13.50509609 | 05H143, 16H145 | HLA-A*02:01 |
| HQLYRASAL (66) | 0.42744 | 9.735569575 | 08H039 | HLA-B*14:02 |
| HTDDIENAKY (67) | 0.08944 | 88.6346121 | 08H039 | HLA-A*01:01 |
| IAAPILHV (68) | 0.45152 | 26.19480016 | 15H023 | HLA-B*51:01 |
| KAFPFHIIF (69) | 0.31792 | 8.766274199 | 07H122 | HLA-B*57:01 |
| KATEYVHSL (70) | 0.14792 | 67.33534262 | 11H008 | HLA-C*06:02 |
| KFSNVTMLF (71) | 0.39376 | 8.927105041 | 05H149 | HLA-A*24:02 |
| KLLEKAFSI (72) | 0.28016 | 20.37743121 | 05H143, 07H122 | HLA-A*02:01 |
| KPMPTKVVF (73) | 0.32808 | 20.74788406 | 11H035 | HLA-B*07:02 |
| NVNRPLTMK (74) | 0.28872 | 12.365496 | 11H008, 16H123 | HLA-A*03:01 |
| REPYELTVPAL (75) | 0.39344 | 8.664178443 | 08H039 | HLA-B*40:01 |
| SEAEAAKNAL (76) | 0.19208 | 15.84619972 | 08H039 | HLA-B*40:01 |
| SLWGQPAEA (77) | 0.89872 | 249.8139721 | 15H063, 16H145 | HLA-A*02:01 |
| SPADHRGYASL (78) | 0.31104 | 28.5105523 | 11H035, 16H123 | HLA-B*07:02 |
| SPQSAAAEL (79) | 0.32288 | 59.61837148 | 11H035, 16H123 | HLA-B*07:02 |
| SPVVHQSL (80) | 0.4292 | 52.97700997 | 11H035 | HLA-B*07:02 |
| SPYRTPVL (81) | 0.4632 | 8.927639423 | 07H063, 11H035 | HLA-B*07:02 |
| SVFAGVVGV (82) | 0.112 | 8.69977003 | 05H143, 16H145 | HLA-A*02:01 |
| SYSPAHARL (83) | 0.2644 | 8.83718913 | 11H035, 16H123 | HLA-C*07:02 |
| THGSEQLHL (84) | 0.3024 | 3.293357687 | 07H141 | HLA-B*38:01 |
| TQAPPNVVL (85) | 0.44232 | 11.85852997 | 15H013 | HLA-C*07:02 |
| VLVPYEPPQV (86) | 0.48944 | 641.852 | 07H122 | HLA-A*02:01 |
| VSFPDVRKV (87) | 0.2852 | 9.647921856 | 05H143 | HLA-C*06:02 |
| VVFDKSDLAKY (88) | 0.16416 | 9.216067541 | 07H060 | HLA-A*29:02 |
| YSHHSGLEY (89) | 0.246 | 91.16567732 | 08H053 | HLA-A*01:01 |
| YYLDWIHHY (90) | 0.29696 | 21.43744849 | 15H013, 16H123 | HLA-C*07:02 |
| SVYKYLKAK (91) | 0.19256 | 8068.72 | 11H008, 16H123 | HLA-A*03:01 |
| IYQFIMDRF (92) | 0.25448 | 86.11916983 | 16H123 | HLA-A*24:02 |
| GTLQGIRAW (93) | 0.37888 | 1098.148 | 05H143, 11H008 | HLA-B*57:01 |
| AQKVSVGQAA (94) | 0.24528 | 2.691338811 | 12H172 | HLA-B*15:01 |
| LYPSKLTHF (95) | 0.22144 | 5.627619975 | 12H172 | HLA-A*24:02 |
| ATQNTIIGK (96) | 0.22592 | 2.541628072 | 08H053 | HLA-A*11:01 |
| AQDIILQAV (97) | 0.34352 | 230.14 | 08H039 | HLA-C*08:02 |
| PPRPLGAQV (98) | 0.4844 | 14.35433865 | 07H063 | HLA-B*07:02 |
| FNVALNARY (99) | 0.29216 | 113.6001075 | 07H060, 11H008 | HLA-A*29:02 |
| GPGSRESTL (100) | 0.41576 | 19.38141684 | 16H123 | HLA-B*07:02 |
| IPHQRSSL (101) | 0.37912 | 61.72974486 | 15H063 | HLA-B*08:01 |
| LTDRIYLTL (102) | 0.34256 | 203.504 | 08H039 | HLA-C*08:02 |
| NLKEKKALF (103) | 0.33608 | 8.968146067 | 07H060 | HLA-B*08:01 |
| VLFGGKVSGA (104) | 0.30776 | 18.89924792 | 07H122 | HLA-A*02:01 |
| VVFPFPVNK (105) | 0.34848 | 1449.166 | 11H035, 16H123 | HLA-A*03:01 |
| SLLIIPKKK (106) | 0.38096 | 5.215809797 | 10H005 | HLA-A*11:01 |
| APGAAGQRL (107) | 0.42808 | 24.61786022 | 07H063, 11H035 | HLA-B*07:02 |
| KLQDKEIGL (108) | 0.24288 | 343.936 | 16H145 | HLA-A*02:01 |
| SLREPQPAL (109) | 0.50512 | 69.20553879 | 15H013 | HLA-C*07:02 |
| TPGRSTQAI (110) | 0.39992 | 8.314753382 | 16H123 | HLA-B*07:02 |
| APRGTAAL (111) | 0.45936 | 17.29079131 | 11H035 | HLA-B*07:02 |
| IASPIALL (112) | 0.44496 | 20.11176673 | 12H172 | HLA-C*03:03 |
| ILFQNSALK (113) | 0.33824 | 16.92592279 | 16H123 | HLA-A*03:01 |
| ILKKNISI (114) | 0.37568 | 22.53624085 | 07H060 | HLA-B*08:01 |
| IPLAVRTI (115) | 0.4452 | 8.644117906 | 15H023 | HLA-B*51:01 |
| LPRNKPLL (116) | 0.36928 | 6.213173676 | 15H023 | HLA-B*51:01 |
| PAPPHPAAL (117) | 0.43568 | 13.04546221 | 07H063 | HLA-C*07:02 |
| SPVVRVGL (118) | 0.45504 | 22.15400444 | 16H123 | HLA-B*07:02 |
| TLNQGINVYI (119) | 0.2868 | 9.544366847 | 14H124 | HLA-A*02:01 |
| RPRGPRTAP (120) | 0.5112 | 29.95425988 | 07H063, 11H035 | HLA-B*07:02 |
| SVQLLEQAIHK (121) | 0.33208 | 56.5275384 | 10H005 | HLA-A*11:01 |
| RTPKNYQHW (122) | 0.2 | 53.71748686 | 07H122 | HLA-B*57:01 |
| ALPVALPSL (123) | 0.55128 | 16.47119814 | 16H145, 14H124 | HLA-A*02:01 |
| SLQILVSSL (124) | 0.414 | 6.710019262 | 08H039 | HLA-B*14:02 |
| ISNKVPKLF (125) | 0.29072 | 19.42035741 | 05H143 | HLA-B*57:01 |
| TVIRIAIVNK (126) | 0.35448 | 43568.4 | 16H123 | HLA-A*03:01 |
| KEIFLELRL (127) | 0.29152 | 90.9946 | 11H008 | HLA-B*44:03 |
| TLRSPGSSL (128) | 0.2984 | 11.27169102 | 11H035 | HLA-B*07:02 |
| TVRGDVSSL (129) | 0.23032 | 9.688689585 | 11H035 | HLA-B*07:02 |
| ALDPLLLRI (130) | 0.4948 | 9.563532996 | 07H122 | HLA-A*02:01 |
| ISLIVTGLK (131) | 0.39056 | 9.244635345 | 15H023 | HLA-A*03:01 |
| KILDVNLRI (132) | 0.28152 | 8.266412385 | 15H023 | HLA-A*02:01 |
| ERVYIRASL (133) | 0.24856 | 7.705323592 | 08H039 | HLA-B*14:02 |
| ILDLESRY (134) | 0.3588 | 16.434 | 07H063 | HLA-A*01:01 |
| KTFVQQKTL (135) | 0.1732 | 261.178 | 07H122 | HLA-B*57:01 |
| LYIKSLPAL (136) | 0.2556 | 19.06117834 | 08H039 | HLA-B*14:02 |
| VLKEKNASL (137) | 0.2932 | 75.9224 | 05H143 | HLA-B*08:01 |
| LGISLTLKY (138) | 0.29328 | 6.31266165 | 07H060 | HLA-A*29:02 |
| DLLPKKLL (139) | 0.40048 | 141.2948 | 07H060 | HLA-B*08:01 |
| HSLISIVYL (140) | 0.25104 | 80.1734 | 16H145 | HLA-C*03:03 |
| IAGALRSVL (141) | 0.4288 | 221.362 | 08H039 | HLA-B*14:02 |
| IGNPILRVL (142) | 0.5164 | 95.0262 | 05H143, 07H122 | HLAC*07:01, HLA-C*06:02 |
| IYAPHIRLS (143) | 0.32096 | 230.392 | 05H143 | HLA-C*07:01 |
| LRSQILSY (144) | 0.28312 | 33.62998983 | 15H080 | HLA-B*27:05 |
| RYLANKIHI (145) | 0.16208 | 64.5122 | 05H149 | HLA-A*24:02 |
| SLLSGLLRA (146) | 0.5932 | 308.14 | 07H122 | HLA-A*02:01 |
| SRIHLVVL (147) | 0.4008 | 60.2848 | 05H143 | HLA-B*08:01 |
| SSSPVRGPSV (148) | 0.37792 | 113.1364 | 05H149 | HLA-A*68:02 |
| STFSLYLKK (149) | 0.18904 | 98.973 | 10H005 | HLA-A*11:01 |
| SLDLLPLSI (150) | 0.39032 | 17.23616137 | 07H141, 08H039 | HLA-C*05:01, HLA-A*02:01 |
| VTDLLALTV (151) | 0.42864 | 10.81032029 | 08H039 | HLA-A*01:01 |
| RTQITKVSLKK (152) | 0.21872 | 2051.04 | 08H053 | HLA-A*11:01 |
| ILRSPLKW (153) | 0.41552 | 56.7804 | 07H122 | HLA-B*57:01 |
| LSTGHLSTV (154) | 0.32176 | 45.7274 | 07H122 | HLA-C*06:02 |
| TVEEYLVNI (155) | 0.24264 | 1.911770046 | 15H063 | HLA-C*07:01 |
| QIKTKLLGSL (156) | 0.3024 | 1.404844942 | 07H060 | HLA-B*08:01 |
| LPSFSHFLLL (157) | 0.38896 | 2.7505537 | 07H063 | HLA-B*07:02 |
| CLRIGPVTL (158) | 0.54248 | 2.362675236 | 11H035 | HLA-C*07:02 |
| HVSDGSTALK (159) | 0.20528 | 2.419142618 | 15H023 | HLA-A*03:01 |
| IAYSVRALR (160) | 0.38472 | 3.755291737 | 11H035 | HLA-A*03:01 |
| PRGFLSAL (161) | 0.45264 | 4.511422509 | 07H063 | HLA-B*07:02 |
| ISSWLISSL (162) | 0.378 | 4.706302579 | 08H039 | HLA-B*14:02 |
| IPLNPFSSL (163) | 0.3876 | 2.67504273 | 16H123 | HLA-B*07:02 |
| LSDRQLSL (164) | 0.42816 | 1.037298278 | 07H060 | HLA-A*01:01 |
| LSHPAPSSL (165) | 0.4132 | 2.060547234 | 07H063 | HLA-A*30:01 |
| LRKAVDPIL (166) | 0.32448 | 3.901328114 | 07H063 | HLA-C*06:02 |
| ILLEEQSLI (167) | 0.4304 | 4.359584524 | 16H145 | HLA-A*02:01 |
| LTSISIRPV (168) | 0.39904 | 1.503160093 | 08H039 | HLA-A*02:01 |
| TISECPLLI (169) | 0.52632 | 1.643501465 | 08H039 | HLA-A*02:01 |
| TLKLKKIFF (170) | 0.23936 | 2.527260887 | 05H143 | HLA-B*08:01 |
| ILLSNFSSL (171) | 0.33432 | 1.032824295 | 15H023 | HLA-A*02:01 |
| LGGAWKAVF (172) | 0.38584 | 1.397521024 | 15H013 | HLA-B*57:03 |
| LSASHLSSL (173) | 0.37704 | 2.203674156 | 07H141 | HLA-C*12:03 |
| AGDIIARLI (174) | 0.40696 | 1.817220719 | 08H039 | HLA-C*08:02 |
| DRGILRNLL (175) | 0.46584 | 1.753521623 | 08H039 | HLA-B*14:02 |
| GLRLIHVSL (176) | 0.37616 | 2.738686931 | 08H039 | HLA-B*14:02 |
| GLRLLHVSL (177) | 0.388 | 3.327626689 | 08H039 | HLA-B*14:02 |
| LHNEKGLSL (178) | 0.23976 | 1.21912196 | 05H143 | HLA-C*07:01 |
| LPSFSRPSGII (179) | 0.38304 | 2.058642372 | 07H063 | HLA-B*07:02 |
| LSSRLPLGK (180) | 0.32344 | 3.016124162 | 11H008 | HLA-A*03:01 |
| MIGIKRLL (181) | 0.4116 | 2.454205925 | 05H143 | HLA-B*08:01 |
| NLKKREIL (182) | 0.42808 | 1.722302438 | 07H060 | HLA-B*08:01 |
| RMVAYLQQL (183) | 0.41624 | 1.99560151 | 12H172 | HLA-A*02:01 |
| SPARALPSL (184) | 0.51256 | 1.404210958 | 16H123 | HLA-B*07:02 |
| TVPGIQRY (185) | 0.39184 | 1.304086581 | 14H124 | HLA-A*26:01 |
| VSRNYVLLI (186) | 0.24664 | 4.908161328 | 07H060 | HLA-C*07:01 |
| LTVPLSVFW (187) | 0.40472 | 1.193403976 | 05H143 | HLA-B*57:01 |
| KLNQAFLVL (188) | 0.35824 | 1.82047525 | 15H080 | HLA-A*02:01 |
| RLVSSTLLQK (189) | 0.32288 | 1.091198313 | 11H035 | HLA-A*03:01 |
| LPSHSLLI (190) | 0.39184 | 2.067938041 | 15H023 | HLA-B*51:01 |

Biotypes were attributed manually upon examination of the peptide coding sequence at the indicated genomic position. Immunogenicity scores were computed with Repitope. HLA alleles correspond to the most likely of presenting the peptide in the given sample, as predicted by netMHC4.0. Synthetic peptide validations were performed only on TSAs^{hi}.

To assess the robustness of MOI identifications, the observed mean retention time (RT) of a given peptide was correlated against the two best-in-class metrics for validation of MAPs identified with high-throughput MS: the RT calculated by the DeepLC algorithm (Bouwmeester et al., 2020) and the hydrophobicity index assessed with SSRcalc (Krokhin, 2006), both predicted based on peptide sequences. This showed that RT distribution of non-canonical MOIs was well correlated to predictions and was not significantly different (F-test) from the distribution of canonical proteome-derived peptides, supporting their correct identification (FIG. 3G). Finally, all MS database searches (initially performed with the PEAKS software) were repeated with the Comet algorithm. The percentage of re-identification showed no significant difference between non-canonical MOIs and canonical peptides (FIG. 3H, left panel). Among MOIs, 52 out of 58 TSAs^{hi} (90%) were re-identified (FIG. 3H, right panel). This major overlap between MAPs identified by two disparate search engines further supports the robustness of non-canonical MOI identification.

### Example 5: TSAs^{hi} are Immunogenic MAPs Deriving Mainly From the Translation of Introns

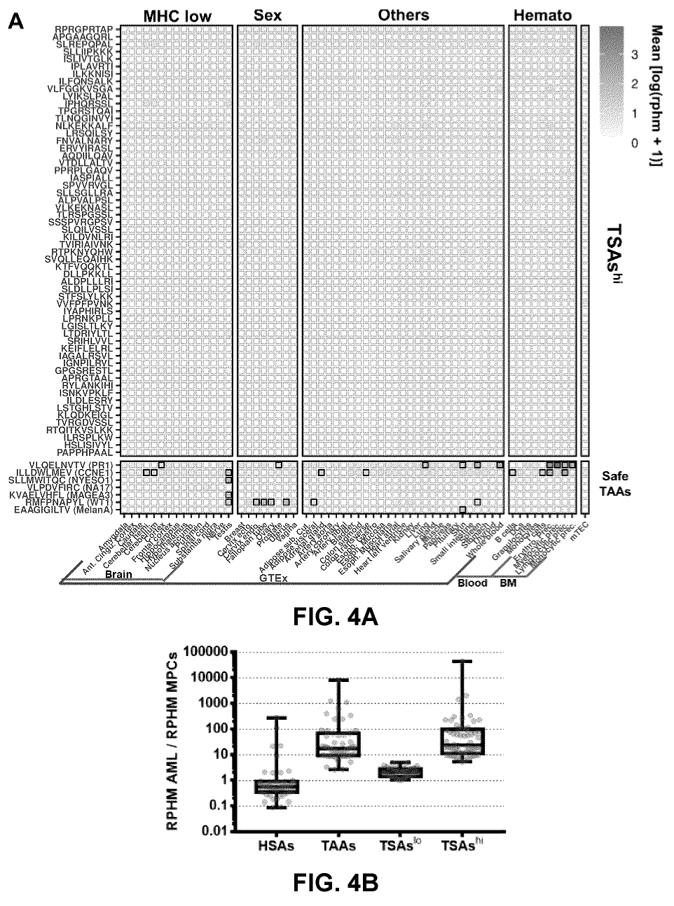

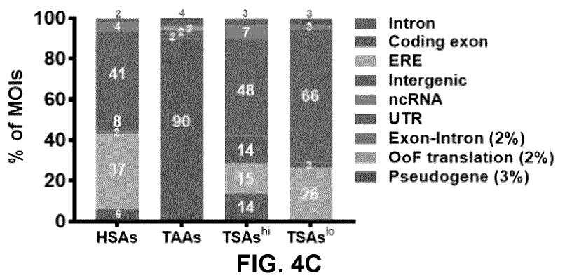

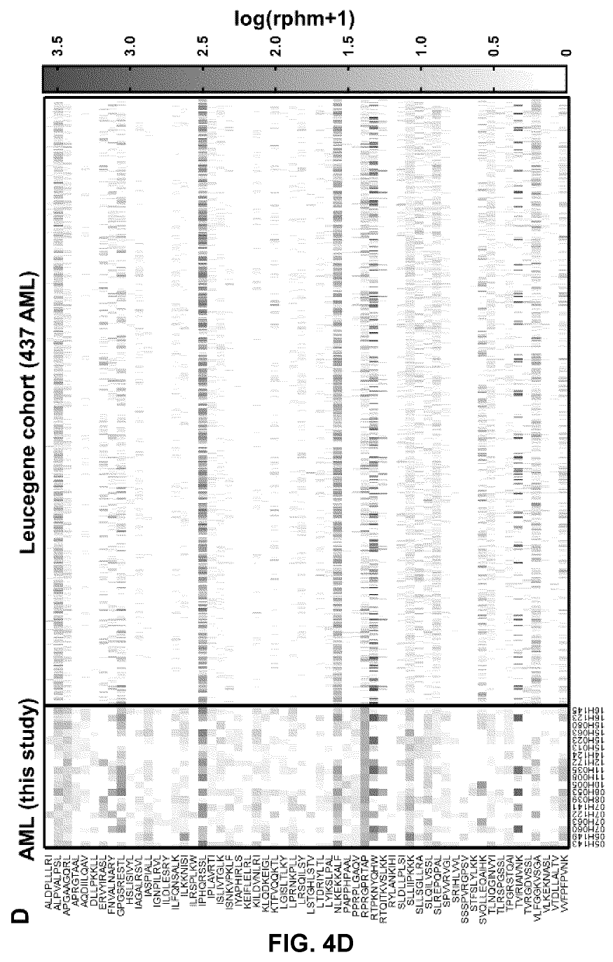

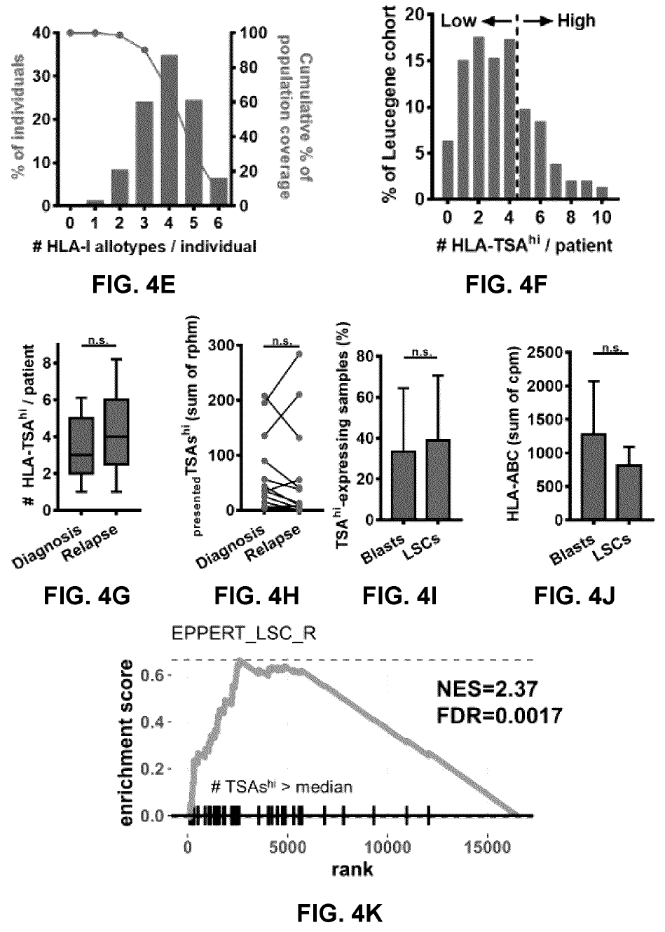

The combination of the four TSA-discovery approaches results yielded a total of 47 HSAs, 49 TAAs, 36 TSAs^{lo} and 58 TSAs^{hi} (without redundancies). Key features of all MOIs are listed in Table 2. By definition, TSAs were expressed below threshold in all organs (from GTEx) as well as in mTECs and normal hematopoietic cells (FIG. 4A). Importantly, expression of TSAs^{hi}-coding RNAs in normal tissues was systematically inferior to expression of TAAs previously used in clinical trials without off-target toxicity (Chapuis et al., 2019; He et al., 2020; Legat et al., 2016; Qazilbash et al., 2017). Consistent with this, none of the TSA^{hi} is present in the HLA Ligand Atlas which contains human MAPs identified in 29 non-malignant tissues, https://www.biorxiv.org/content/10.1101/778944v1). This supports the safety of targeting TSA^{lo} and 58 TSAs^{hi} (without redundancies). The TAAs presented elevated expression in at least one normal tissue while HSAs expression was restricted to the hematopoietic compartment. Comparison of FC between AML specimens and MPCs showed that TSAs^{hi} presented the highest overexpression together with TAAs (median of 22-fold) while HSAs were expressed at the highest levels in healthy cells (median of 0.6-fold) (FIG. 4B). Altogether these results show that TSAs^{hi} combine the advantages of both worlds: specificity / safety of TSAs and overexpression of TAAs.

The TSAs identified mostly derived from allegedly non-coding regions of the genome as only 13% of them derived from canonical protein exons and 58% of them derived from introns (FIG. 4C). Not a single one derived from mutations, consistent with AML low mutation burden (Lawrence et al., 2013). TAAs mainly derived from protein coding exons while HSAs origins were also dominated by non-coding regions, in agreement with previous studies reporting tissue-specific intron retention and ERE expression patterns (Middleton et al., 2017; Larouche et al., 2020). While eight TSAs^{hi} derived from canonical protein-coding genes, they may be considered as safe targets given their low expression in normal tissues relative to safe TAAs (FIG. 4A). Supporting their relevance as therapeutic targets, three of them derive from known AML biomarkers (LTBP1, MYCN and PLPPR3) and the other five have unknown functions or are involved in proliferation, differentiation or drug resistance (Table 3).

TABLE 3

Characteristics of canonical protein-coding genes from which derived TSAs^{hi} have been identified

| | | | |
|---|---|---|---|
| Gene symbol | Full name | Ensembl ID | Cancer-relevant note |
| LTBP1 | latent transforming growth factor beta binding protein 1 | ENSG00000049323 | Facilitates secretion of latent TGF-β, overexpressed in AML (Wilson et al., 2006) |
| MYCN | MYCN proto-oncogene, bHLH transcription factor | ENSG00000134323 | Leukemogenic function, overexpressed in AML (Liu et al., 2017; Wilson et al., 2006) |
| PLPPR3 | Phospholipid phosphatase related 3 | ENSG00000129951 | Target of ANPA32 leukemogenic gene, overexpressed in AML (Yang et al., 2018) |
| GCSAML | Germinal center associated signaling and motility like | ENSG00000169224 | Association with proliferation, methylation of this gene is altered in hematopoietic malignancies (de Sá Machado Araújo et al., 2018) |
| DNAH10 | Dynein axonemal heavy chain 10 | ENSG00000197653 | Formation of axonemes of primary cilia, associated with proliferation and differentiation (Lagus et al., 2019) |
| ST8SIA6 | ST8 alpha-N-acetyl-neuraminide alpha-2,8-sialyltransferase 6 | ENSG00000148488 | Multidrug resistance (Zhang et al., 2015) |
| LINC01835 | C-type lectin domain family 4 member O | ENSG00000267453 | Unknown function |
| MFSD2B | Major facilitator superfamily domain containing 2B | ENSG00000205639 | Putative epigenetic regulator in myeloid progenitor cells (Johnson et al., 2015) |

The therapeutic value of a TSA depends in part on the extent to which it is shared by patients. To evaluate TSA^{hi} sharing among primary AMLs, the Leucegene cohort which includes RNA-seq data from purified AML blasts for 437 patients (Lavallee et al., 2015; Macrae et al., 2013; Pabst et al., 2016), was analyzed. Because most MAPs can be presented by different HLA allotypes, the presentation of the identified TSAs^{hi} was first evaluated by taking promiscuous binders into account. By using the MHCcluster tool, which clusters together HLA alleles presenting similar epitopes (Thomsen et al., 2013), the full set of HLA allotypes capable of presenting individual TSA^{hi} could be extrapolated (Table 4). Based on these data, it was shown that among the world’s population, 99.92% of individuals carry ≥1 HLA-I allotype capable of presenting one TSA^{hi}. Next, it was considered that an individual TSA^{hi} was present in a given AML sample only when the TSA coding transcript was expressed and the patient had an HLA allotype that could present this TSA. Based on these criteria, it could be predicted that in the Leucegene cohort, the median number of TSA^{hi} per patient was four, and 93.6% of patients would present at least one TSA^{hi} (FIG. 4F).

TABLE 4

List of HLA alleles capable of presenting similar peptides (promiscous binders) as predicted by MHCcluster.

| | |
|---|---|
| HLA allele | Other alleles capable of presenting similar peptides (promiscuous binders) |
| HLA-A*01:01 | |
| HLA-A*02:01 | HLA-A*02:05 HLA-A*02:06 HLA-A*02:07 |
| HLA-A*02:05 | HLA-A*02:05 HLA-A*02:06 HLA-A*02:07 |
| HLA-A*02:06 | HLA-A*02:05 HLA-A*02:06 HLA-A*02:07 |
| HLA-A*02:07 | HLA-A*02:01 HLA-A*02:05 HLA-A*02:06 |
| HLA-A*03:01 | HLA-A*11:01 |
| HLA-A*11:01 | HLA-A*03:01 HLA-A*31:01 HLA-A*68:01 |
| HLA-A*23:01 | HLA-A*24:02 |
| HLA-A*24:02 | HLA-A*23:01 |
| HLA-A*25:01 | HLA-A*26:01 HLA-A*66:01 HLA-B*15:02 |
| HLA-A*26:01 | HLA-A*25:01 HLA-A*66:01 |
| HLA-A*29:02 | HLA-A*30:02 HLA-B*15:02 |
| HLA-A*30:01 | |
| HLA-A*30:02 | HLA-A*29:02 |
| HLA-A*31:01 | HLA-A*11:01 HLA-A*33:01 HLA-A*33:03 HLA-A*68:01 |
| HLA-A*32:01 | HLA-B*57:01 HLA-B*58:01 |
| HLA-A*33:01 | HLA-A*31:01 HLA-A*33:03 HLA-A*68:01 |
| HLA-A*33:03 | HLA-A*31:01 HLA-A*33:01 HLA-A*68:01 |
| HLA-A*66:01 | HLA-A*25:01 HLA-A*26:01 |
| HLA-A*68:01 | HLA-A*11:01 HLA-A*31:01 HLA-A*33:01 HLA-A*33:03 |
| HLA-A*68:02 | |
| HLA-B*07:02 | HLA-B*35:02 HLA-B*35:03 HLA-B*55:01 HLA-B*56:01 |
| HLA-B*08:01 | |
| HLA-B*14:02 | HLA-B*39:01 |
| HLA-B*15:01 | HLA-B*15:02 HLA-B*15:03 HLA-B*46:01 |
| HLA-B*15:02 | HLA-A*25:01 HLA-A*29:02 HLA-B*15:01 HLA-B*15:03 HLA-B*15:18 HLA-B*35:01 HLA-B*46:01 |
| HLA-B*15:03 | HLA-B*15:01 HLA-B*15:02 HLA-B*15:18 |
| HLA-B*15:18 | HLA-B*15:02 HLA-B*15:03 |
| HLA-B*18:01 | HLA-B*40:01 HLA-B*44:02 HLA-B*44:03 HLA-B*45:01 |
| HLA-B*27:02 | HLA-B*27:05 HLA-C*06:02 HLA-C*07:01 |
| HLA-B*27:05 | HLA-B*27:02 |
| HLA-B*35:01 | HLA-B*15:02 HLA-B*35:02 HLA-B*35:03 HLA-B*53:01 |
| HLA-B*35:02 | HLA-B*07:02 HLA-B*35:01 HLA-B*35:03 HLA-B*51:01 HLA-B*53:01 HLA-B*55:01 HLA-B*56:01 |
| HLA-B*35:03 | HLA-B*07:02 HLA-B*35:01 HLA-B*35:03 HLA-B*51:01 HLA-B*53:01 HLA-B*55:01 HLA-B*56:01 |
| HLA-B*38:01 | HLA-B*39:01 |
| HLA-B*39:01 | HLA-B*14:02 HLA-B*38:01 |
| HLA-B*40:01 | HLA-B*18:01 HLA-B*40:02 HLA-B*41:02 HLA-B*44:02 HLA-B*44:03 HLA-B*45:01 |
| HLA-B*40:02 | HLA-B*40:01 HLA-B*41:02 HLA-B*44:02 HLA-B*44:03 HLA-B*45:01 |
| HLA-B*41:02 | HLA-B*40:01 HLA-B*40:02 HLA-B*44:02 HLA-B*44:03 HLA-B*45:01 |
| HLA-B*44:02 | HLA-B*18:01 HLA-B*40:01 HLA-B*40:02 HLA-B*41:02 HLA-B*44:03 HLA-B*45:01 |
| HLA-B*44:03 | HLA-B*18:01 HLA-B*40:01 HLA-B*40:02 HLA-B*41:02 HLA-B*44:02 HLA-B*45:01 |
| HLA-B*45:01 | HLA-B*18:01 HLA-B*40:01 HLA-B*40:02 HLA-B*41:02 HLA-B*44:02 HLA-B*44:03 |
| HLA-B*46:01 | HLA-B*15:01 HLA-B*15:02 HLA-C*03:02 HLA-C*03:03 HLA-C*03:04 HLA-C*08:01 HLA-C*12:02 HLA-C*12:03 HLA-C*16:01 |
| HLA-B*51:01 | HLA-B*35:02 HLA-B*35:03 HLA-B*52:01 HLA-B*53:01 HLA-B*55:01 HLA-B*56:01 |
| HLA-B*52:01 | HLA-B*51:01 |
| HLA-B*53:01 | HLA-B*35:01 HLA-B*35:02 HLA-B*35:03 HLA-B*51:01 |
| HLA-B*55:01 | HLA-B*07:02 HLA-B*35:02 HLA-B*35:03 HLA-B*51:01 HLA-B*56:01 |
| HLA-B*56:01 | HLA-B*07:02 HLA-B*35:02 HLA-B*35:03 HLA-B*51:01 HLA-B*55:01 |
| HLA-B*57:01 | HLA-A*32:01 HLA-B*58:01 |
| HLA-B*58:01 | HLA-A*32:01 HLA-B*57:01 |
| HLA-C*03:02 | HLA-B*46:01 HLA-C*03:03 HLA-C*03:04 HLA-C*08:01 HLA-C*12:02 HLA-C*12:03 HLA-C*16:01 |
| HLA-C*03:03 | HLA-B*46:01 HLA-C*03:02 HLA-C*03:04 HLA-C*08:01 HLA-C*08:02 HLA-C*12:02 HLA-C*12:03 HLA-C*15:02 HLA-C*16:01 |
| HLA-C*03:04 | HLA-B*46:01 HLA-C*03:02 HLA-C*03:04 HLA-C*08:01 HLA-C*08:02 HLA-C*12:02 HLA-C*12:03 HLA-C*15:02 HLA-C*16:01 |
| HLA-C*04:01 | HLA-C*07:02 HLA-C*14:02 |
| HLA-C*05:01 | HLA-C*08:01 HLA-C*08:02 |
| HLA-C*06:02 | HLA-B*27:02 HLA-C*07:01 HLA-C*07:02 |
| HLA-C*07:01 | HLA-B*27:02 HLA-C*06:02 HLA-C*07:02 HLA-C*14:02 |
| HLA-C*07:02 | HLA-C*04:01 HLA-C*06:02 HLA-C*07:01 HLA-C*14:02 |
| HLA-C*08:01 | HLA-B*46:01 HLA-C*03:02 HLA-C*03:03 HLA-C*03:04 HLA-C*05:01 HLA-C*08:02 HLA-C*12:02 HLA-C*12:03 HLA-C*15:02 HLA-C*16:01 |
| HLA-C*08:02 | HLA-C*03:03 HLA-C*03:04 HLA-C*05:01 HLA-C*08:01 HLA-C*15:02 |
| HLA-C*12:02 | HLA-B*46:01 HLA-C*03:02 HLA-C*03:03 HLA-C*03:04 HLA-C*08:01 HLA-C*12:03 HLA-C*15:02 HLA-C*16:01 |
| HLA-C*12:03 | HLA-B*46:01 HLA-C*03:02 HLA-C*03:03 HLA-C*03:04 HLA-C*08:01 HLA-C*12:03 HLA-C*15:02 HLA-C*16:01 |
| HLA-C*14:02 | HLA-C*04:01 HLA-C*07:01 HLA-C*07:02 HLA-C*16:01 |
| HLA-C*15:02 | HLA-C*03:03 HLA-C*03:04 HLA-C*08:01 HLA-C*08:02 HLA-C*12:02 HLA-C*12:03 |
| HLA-C*16:01 | HLA-B*46:01 HLA-C*03:02 HLA-C*03:03 HLA-C*03:04 HLA-C*08:01 HLA-C*12:02 HLA-C*12:03 HLA-C*14:02 |

When comparing the number of TSA^{hi} in AML samples analyzed at the time of initial diagnosis vs. at relapse (unmatched samples), no difference between both groups was found (FIG. 4G). No difference were also found when comparing the RNA expression of the TSAs^{hi} that could be presented by patients HLA alleles in matched samples of AML blasts obtained at time of diagnosis and at relapse after allogeneic hematopoietic cell transplantation in another study (Toffalori et al., 2019) (FIG. 4H). As leukemic stem cells (LSCs) are the main mediators of relapse (Shlush et al., 2017), TSAs^{hi} and HLA RNA expression were also evaluated in sorted LSCs vs blasts RNA-seq data obtained from another study (Corces et al., 2016) and found no difference between the two cell populations (FIGS. 4I-J). Nonetheless, by using gene set enrichment analysis (GSEA), it was found that patients expressing high numbers of TSAs^{hi} also expressed higher levels of a well-established LSC gene signature (Eppert et al., 2011) (FIG. 4K). Altogether, these results further support the high immunogenicity of TSAs^{hi} and demonstrate that they could be targeted in virtually all AML patients, either at diagnosis or relapse. It may thus be concluded that immune targeting of TSA^{hi} could be envisioned at any stage of the disease and would have the potential to eliminate LSCs.

### Example 6: Presentation of Numerous TSAs^{hi} Correlates With Better Survival

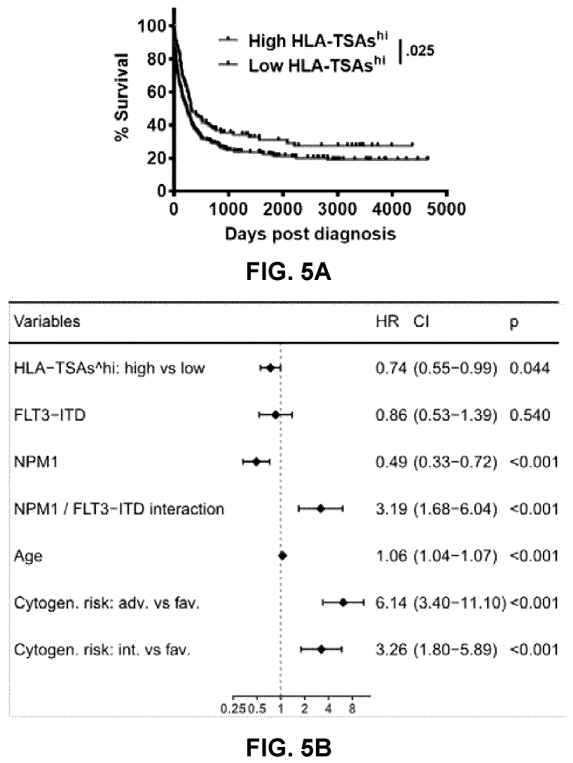

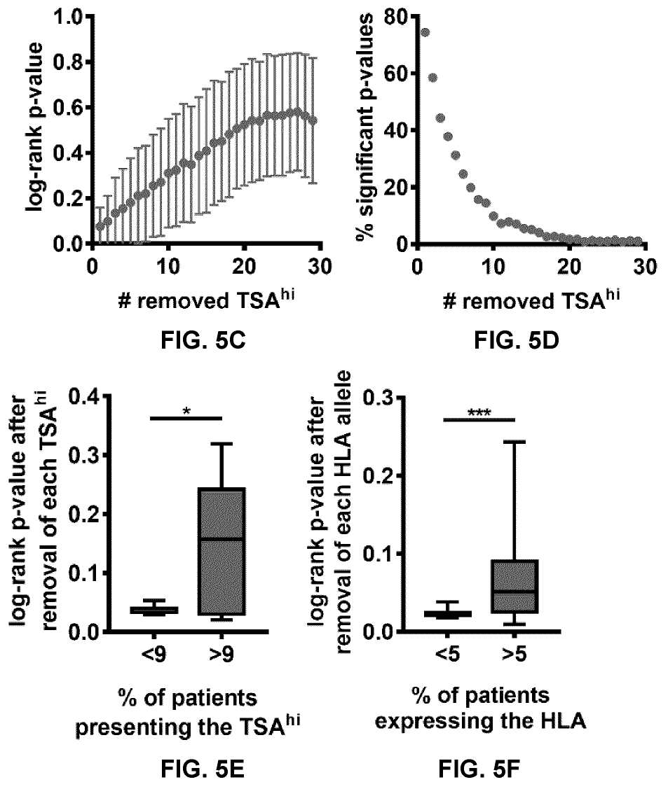

Next, the repercussion of TSAs^{hi} presentation at diagnosis on patient survival was examined. Strikingly, patients expressing the highest numbers (upper quartile) of TSAs^{hi} presented a significantly better survival than the rest of the cohort (FIG. 5A). The survival advantage linked to presentation of multiple TSA^{hi} remained significant in multivariate analysis, together with other known prognostic factors such as age, cytogenetic risk, NPM1 and FLT3-ITD mutations (FIG. 5B). Importantly, the same comparison performed independently of HLA _{pred}presentation showed no difference between high and low expressors (FIGS. 11A, B), meaning that the protective effect of TSA^{hi} was HLA-restricted. The same analysis performed on TAA, HSA or TSA^{lo} showed no significant impact on survival (FIGS. 11C-H). These data suggest that TSA^{hi} are sufficiently immunogenic to elicit spontaneous anti-AML immune responses.

To demonstrate that the survival advantage provided by TSAs^{hi} resulted from their cumulative HLA _{pred}presentation, the log-rank p-value of high vs low TSAs^{hi} patients was computed after random removal of increasing numbers of TSAs^{hi} (1 to 29 out of the 58) from the analysis (1000 random permutations / number). Stochastic removal of increasing numbers of TSA^{hi} rapidly led to loss of significant survival advantage for high expressors (upper quartile of HLA-TSAs^{hi} counts) relative to low expressors (all the other patients) (FIGS. 5C-D). This incremental decrease in survival advantage with subtraction of individual TSAs^{hi} suggests that the majority of TSAs^{hi} contribute to this survival advantage. TSAs^{hi} presented by greater proportions of patients had the greatest impact on p-values (FIG. 5E). Likewise, removal of common HLA alleles (shared by more than 5% of patients) from the log-rank analysis had a greater impact on p-values than removal of low frequency alleles (FIG. 5F). Altogether these data demonstrate the HLA-restricted benefit of TSAs^{hi} presentation on patient survival. In the next experiments, the most parsimonious explanation for the survival advantage linked to TSAs^{hi} presentation is examined: TSAs^{hi} elicit spontaneous protective anti-AML immune responses.

### Example 7: TSAs^{hi} Presentation Triggers Cytotoxic T Cell Responses

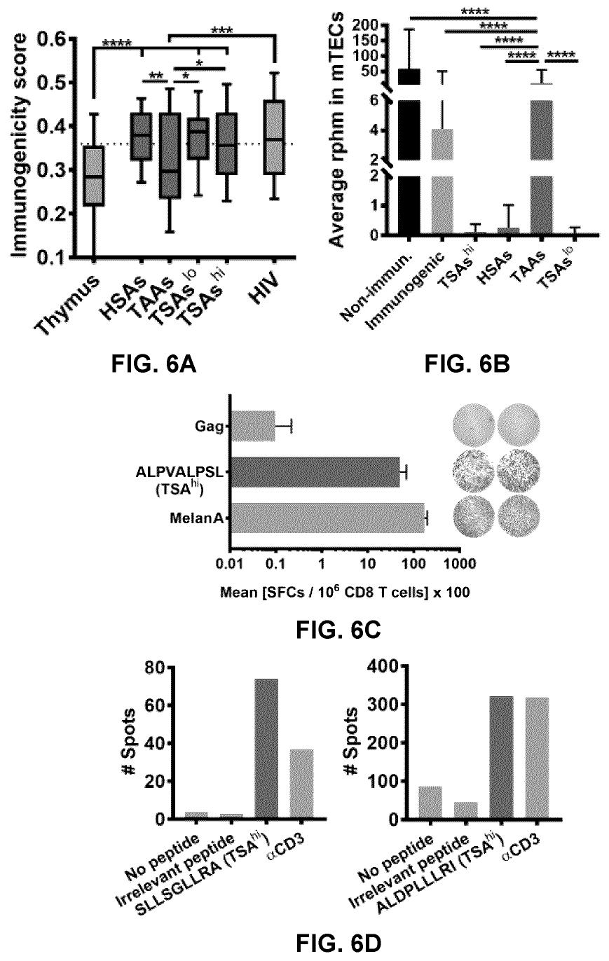

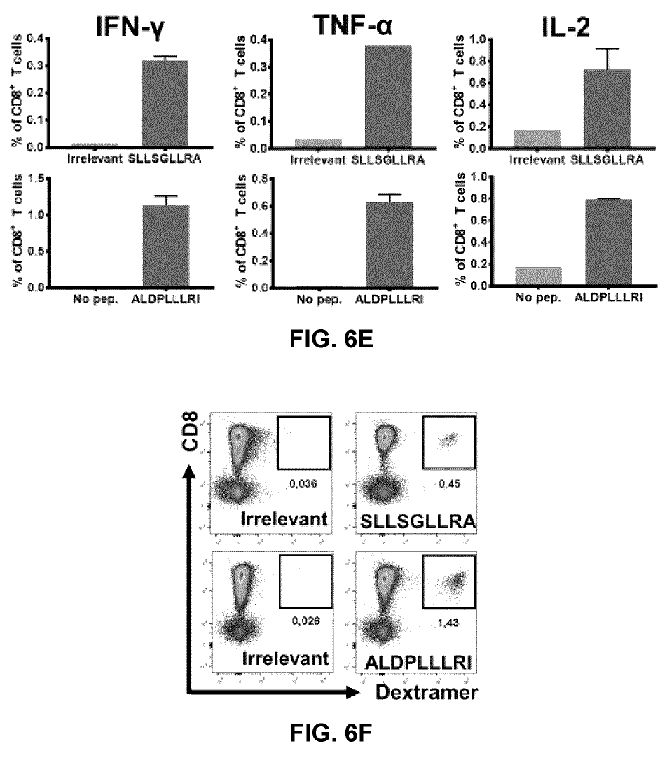

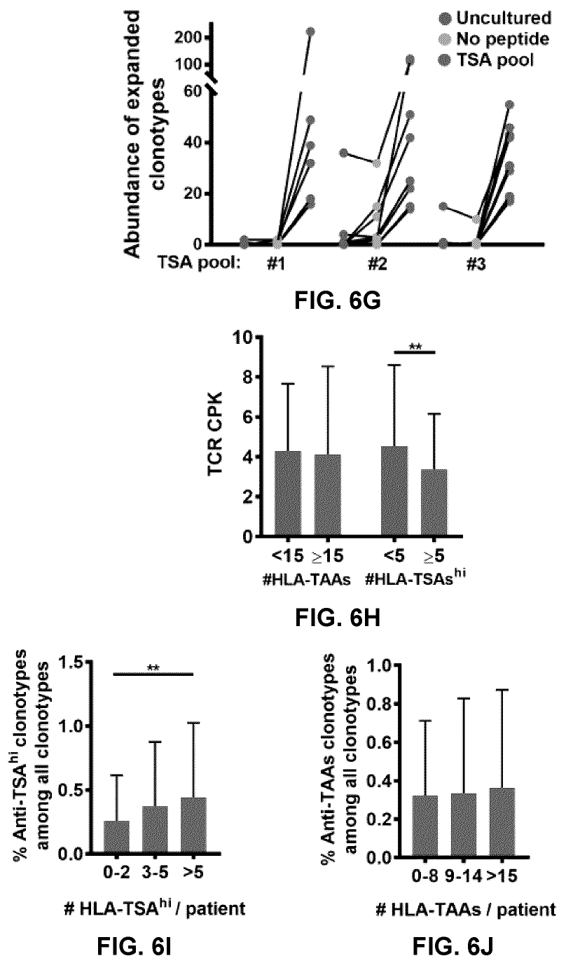

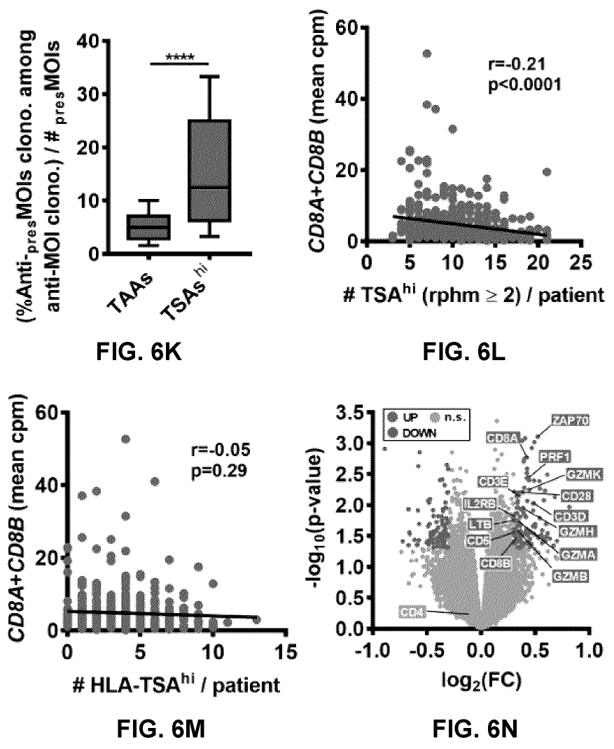

As a prelude to the assessment of immunogenicity (i.e. their ability to induce an immune response) of TSAs^{hi} and other MOIs, Repitope, a machine learning algorithm which relies on public TCR databases to predict a probability of T-cell response (Ogishi and Yotsuyanagi, 2019), was used. Using MAPs presented by thymic epithelial cells (Adamopoulou et al., 2013) or deriving from the HIV as negative and positive controls respectively, Repitope predictions indicated that TAAs were mostly non-immunogenic while the three other groups of MOIs were as immunogenic as HIV peptides (FIG. 6A). Accordingly, TAAs presented a high expression in mTECs (-12.1 rphm) relative to the three other groups and relative to a set of 1411 MAPs reported as immunogenic in IEDB (FIG. 6B). Non-TAAs MOIs all presented a very low RNA expression in mTECs even relative to other immunogenic peptides, supporting their immunogenicity. To validate Repitope predictions, in vitro T-cell assays were performed beginning with the HLA-A*02:01-presented TSAs^{hi} predicted to be most highly immunogenic: ALPVALPSL. As a positive control in IFN-γ ELISpot, the ELAGIGILTV epitope was used because it is one of the most immunogenic human MAPs (Dutoit et al., 2002; Hesnard et al., 2016). The immunogenicity of ALPVALPSL was similar to that of ELAGIGILTV (FIG. 6C). ELISpot of two other promising TSAs^{hi} also supported their immunogenicity (FIG. 6D). For these TSAs^{hi}, cytokine secretion assays and dextramer staining were also performed, which confirmed the ELISpot result and supported the specificity of the immune response (FIGS. 6E-F). To further demonstrate that TSAs^{hi} can induce spontaneous and specific T-cell clonotypes expansion, a functional expansion of specific T cells (FEST) assay, in which short-term cultures of peripheral blood T cells stimulated with different pools of TSAs^{hi} are analyzed through TCR sequencing (Danilova et al., 2018), was performed. Each pool of 5 tested TSAs^{hi} induced the specific expansion of 9-10 different clonotypes, supporting their spontaneous immunogenicity (FIG. 6G and Table 5).

TABLE 5

Functional expansion of specific T cells (FEST) assay in response to pools of TSAs^{hi}.

| Pool # | TSAs^{hi} in pool | Significantly expanded clonotype (SEQ ID NO:) | FDR | Odds ratio |
|---|---|---|---|---|
| 1 | SLLSGLLRA ALPVALPSL ALDPLLLRI IASPIALL SLDLLPLSI | CSARGDREYEQYF (191) | 4.84 E-52 | Inf |
| 1 | | CASTWAGNSSPLHF (192) | 1.2 E-07 | 18.829 |
| 1 | | CASSQDGIWGAYEQYF (193) | 1.95 E-07 | Inf |
| 1 | | CASSVDAGGNYEQYF (194) | 0.000665 | 12.293 |
| 1 | | CASSLGGQGLSYGYTF (195) | 0.011005 | Inf |
| 1 | | CASSYRPNEQYF (196) | 0.011005 | Inf |
| 1 | | CASSGTDLNQPQHF (197) | 0.032852 | Inf |
| 1 | | CASSSNFEPLHF (198) | 0.032852 | Inf |
| 1 | | CASSWGGSNTGELFF (199) | 0.032852 | Inf |
| 2 | LTDRIYLTL VLFGGKVSGA LGISLTLKY FNVALNARY TLNQGINVYI | CASSEYRALNTEAFF (200) | 4.67 E-30 | 59.789 |
| 2 | | CASSESGTGGQPQHF (201) | 1.14 E-08 | 3.455 |
| 2 | | CASSRTGENTEAFF (202) | 0.003813 | 3.352 |
| 2 | | CASSSTDRQHYGYTF (203) | 0.007052 | 3.764 |
| 2 | | CASSDRTGGSSNEKLFF (204) | 0.008031 | 8.213 |
| 2 | | CATSRSGDSNQPQHF (205) | 0.008031 | 8.213 |
| 2 | | CASSYVLNTEAFF (206) | 0.010284 | 10.837 |
| 2 | | CASRESGQMNEKLFF (207) | 0.015395 | Inf |
| 2 | | CASSDGGEGTYGYTF (208) | 0.028405 | Inf |
| 2 | | CASTSWTGFGPNYGYTF (209) | 0.028405 | Inf |
| 3 | LRSQILSY KILDVNLRI HSLISIVYL KLQDKEIGL AQDIILQAV | CASSADSSLGGYTF (210) | 1.13 E-10 | 45.434 |
| 3 | | CSARDLAGGTYEQYF (211) | 8.08 E-10 | Inf |
| 3 | | CASSPPTGEYEKLFF (212) | 9.54 E-09 | Inf |
| 3 | | CARSFGGFF (213) | 3.78 E-06 | Inf |
| 3 | | CASSLPSGILYEQYF (214) | 3.78 E-06 | Inf |
| 3 | | CASAPGGMPYGYTF (215) | 1.16 E-05 | Inf |
| 3 | | CASSLQDTDYNEQFF (216) | 0.005265 | Inf |
| 3 | | CASSDPGTSGVFTGELFF (217) | 0.009073 | Inf |
| 3 | | CSARLGTGELFF (218) | 0.016806 | Inf |
| 3 | | CASSLGRGYETQYF (219) | 0.019961 | 3.55 |

All TSAs^{hi} that could be presented by the HLA alleles of an HLA-A*02:01, -A*29:02, -B*15:01, -B27:05, -C*01:02, -C*03:04 healthy donor. TCR-seq were made by Adaptive Biotechnologies and raw data were processed with the FEST analysis tool http://www.stat-apps.onc.jhmi.edu/FEST. Are reported here the number of the pool, the TSAs^{hi} present in each pool, the sequence of each clonotype significantly expanded in each pool and the FDR and odds ratio provided by the FEST analysis tool.

Next, because “in vivo veritas”, in-depth analyses of transcriptomic data from the 437 Leucegene patients was performed to evaluate potential in vivo recognition of TSAs^{hi} by T cells. First, the TCR repertoire diversity of T cells was evaluated with the TRUST4 algorithm (Zhang et al., 2019). In contrast to TAAs (that was used here as non-immunogenic controls), the _{pred}presentation of elevated TSAs^{hi} numbers was associated with a reduced TCR repertoire diversity, suggestive of expansion of anti-TSA^{hi} clonotypes (FIG. 6H). To demonstrate the specificity of this expansion, the ERGO algorithm was used to predict MOI-TCR interactions (Springer et al., 2020). At an ERGO probability >80% to identify anti-MOIs clonotypes, patients with high numbers of TSAs^{hi} also had greater frequencies of anti-TSAs^{hi} clonotypes among all detected CDR3 (FIG. 6I). No similar correlation was seen for TAAs (FIG. 6J). Next, the proportion of anti-MOI clonotypes capable of recognizing MOIs _{pred}presented by the respective AML sample (i.e., the frequency of cognate TCR-MOI interactions) was computed. This proportion was normalized according to the number of _{pred}presented MOIs because otherwise presentation of more numerous MOIs would automatically result in the detection of higher proportions of anti-_{pred}presented MOI clonotypes. This showed that TSAs^{hi} _{pred}presentation was associated with dramatically higher frequencies of specific T-cell recognition than TAAs (FIG. 6K).

In light of the anti-TSAs^{hi} T-cell recognition, it was reasoned that TSAs^{hi} _{pred}presentation should be associated with infiltration of activated CD8 T cell. Interestingly, the diversity of TSAs^{hi} transcripts was inversely correlated with the CD8A+CD8B expression in AML samples, while the diversity of their _{pred}presentation was not (FIGS. 6L-M). This suggested that a high diversity of TSAs^{hi} transcripts reflected a slightly higher blast purity in AML samples (as might be expected for TSAs). To circumvent this possible bias and because the number of HLA-TSAs^{hi} is mathematically linked to the number of expressed TSAs^{hi}, the number of _{pred}presented TSAs^{hi} was normalized to the number of expressed TSAs^{hi} transcripts, and differential gene expression was analyzed in patients whose normalized _{pred}presentation was above- vs below-median. Strikingly, among the 123 genes positively associated with TSAs^{hi} _{pred}presentation, several were associated with T-cell activation and cytolysis, including CD8A, CD8B, GZMA, GZMB, IL2RB, PRF1 and ZAP70 (FIG. 6N). Notably, GO terms associated to these 123 genes were exclusively related to T-cell activation and differentiation (FIG. 6O). The CD4 gene was not differentially expressed and no GO term could be significantly associated with downregulated genes. Hence, it was concluded that TSAs^{hi} _{pred}presentation is associated with higher abundance of activated CD8 T cells.

### Example 8: TSAs^{hi} RNA Expression is Associated With Signs of Immunoediting, AML Driver Mutations and Epigenetic Aberrations

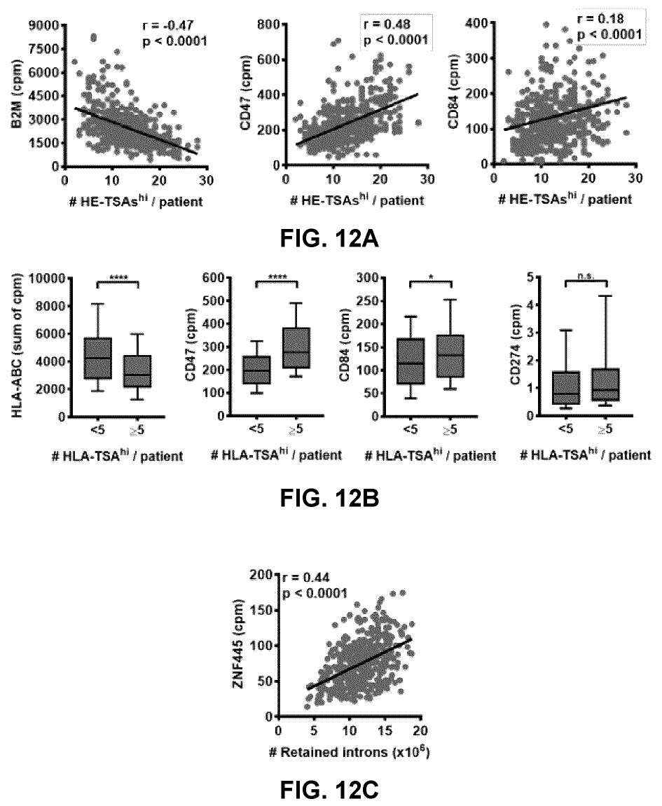

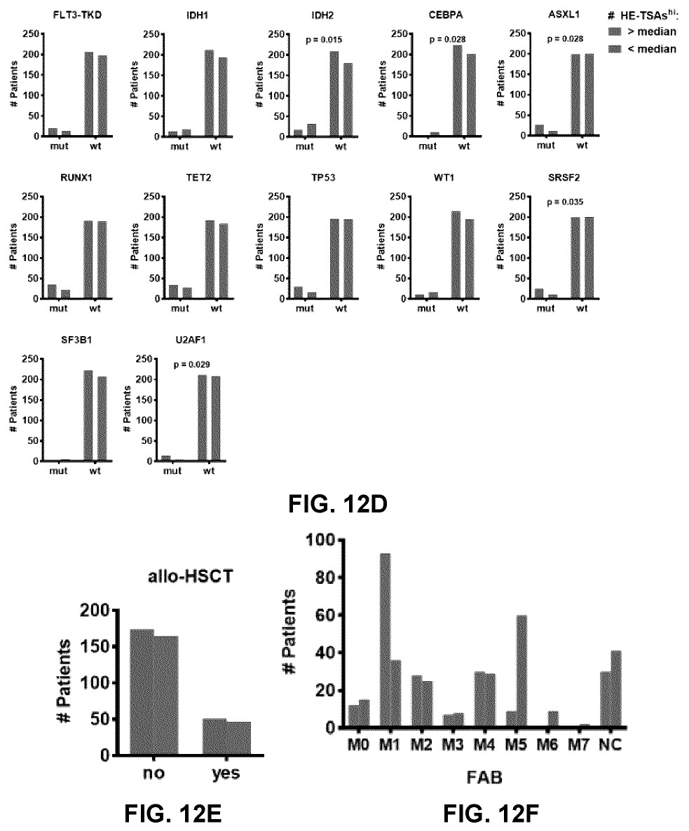

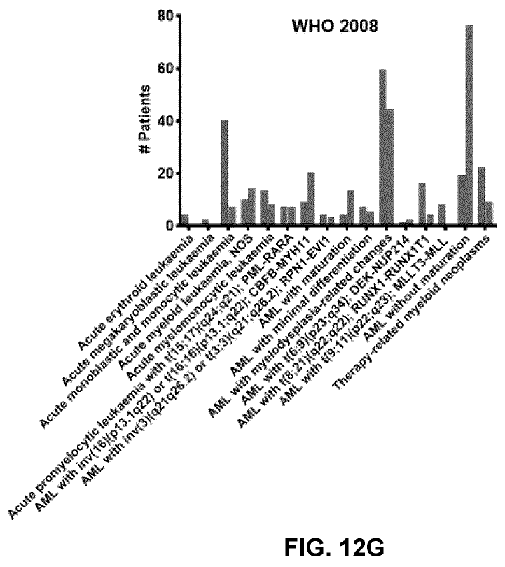

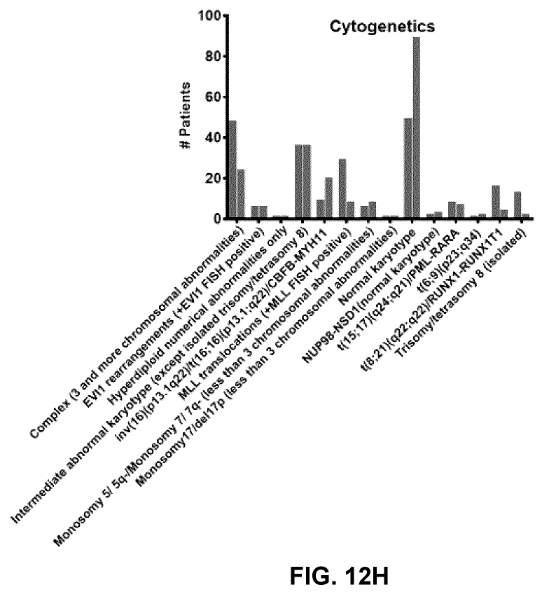

Given the potential therapeutic value of TSA^{hi}, it is desirable to gain insights into their biogenesis. For this analysis, a count of highly expressed TSAs^{hi} (HE-TSAs^{hi}) was attributed to each Leucegene patient, i.e. their count of TSAs^{hi} expressed at levels higher than their median expression across all patients having non-null expression of the given TSAs^{hi}. It was then evaluated whether expression of specific genes could be linked to TSAs^{hi} expression by performing pairwise Pearson correlations between the expression of each protein coding genes and the HE-TSAs^{hi} counts. This showed a consistent inverse correlation between the expression of genes involved in MAP presentation (HLA-A, HLA-B, HLA-C, B2M and NLRC5) and the numbers of HE-TSAs^{hi}, suggesting the occurrence of immunoediting in response to elevated TSAs^{hi} expression (FIGS. 7A and 12A-B). Immunoediting was also supported by the positive correlations with CD47, an immune checkpoint molecule involved in the inhibition of dendritic cell phagocytosis (Majeti et al., 2009), and CD84, which promotes PD-L1 expression by leukemic cells (Lewinsky et al., 2018). Because NPM1 mutations can modulate PD-L1 (CD274) expression (Greiner et al., 2017), NPM1^{mut} and NPM1^{wt} AML patients were analyzed separately. This analysis revealed that NPM1^{wt} patients with above-median HE-TSAs^{hi} counts expressed significantly higher levels of PD-L1 than those with inferior HE-TSAs^{hi} counts (FIG. 7B).

Next, the gene pathways correlated with HE-TSAs counts were analyzed (FIG. 7C and Table 6). Negatively correlated pathways included biological processes involved in cell proliferation (also including transport and cell organization), mitochondrial OXPHOS and proteasomal-mediated protein catabolism. Interestingly, inhibition of mitochondrial activity has been shown to reduce MHC-I expression and could be used as immune escape mechanism by cancer cells (Charni et al., 2010). Similarly, inhibition of protein degradation could lead to lower amounts of peptides for presentation by MHC-I molecules and therefore would reduce the probability of presenting TSAs^{hi} (Tripathi et al., 2016). Finally, the reduction of mitosis-related processes could be a side-effect of MHC-I downregulation, as both processes are regulated by NLRC5 (Wang et al., 2019). Altogether, these data show that TSAs^{hi} expression is linked to various responses that may act as immunoediting mechanisms for AML cells.

In contrast with negatively correlated pathways, positively correlated pathways were restricted to regulation processes (FIG. 7D). Accordingly, 16.1% (vs 2.5% of negatively correlated) of positively correlated genes were transcription factors, which could be directly responsible for the transcription of the TSAs^{hi}. Among them, the best correlated gene was ZNF445 (FIG. 7A), a regulator of genomic imprinting, i.e. an epigenetic process linked to DNA methylation (Takahashi et al., 2019). Since ZNF445 function is dependent on DNA methylation, the possible association between TSAs^{hi} expression and AML mutations typically linked to DNA methylation aberrations was examined. The three most frequent AML driver mutations (NPM1^{mut}, FLT3-ITD and DNMT3A^{mut}) were first tested, and it was found that all three were significantly enriched in patients expressing high HE-TSAs^{hi} counts (above median) (FIG. 7E). Also, patients presenting two or three concomitant mutations had higher numbers of HE-TSAs^{hi} than patients presenting one or none of it (FIG. 7F). Of the 19 AML specimens used in the MS analyses, 12 presented either a FLT3-ITD or NPM1 mutation. Regarding other frequent AML mutations, it was found that IDH2 and biallelic CEBPA mutations were also positively associated with elevated HE-TSAs^{hi} counts while ASXL1, SRSF2 and U2AF1 mutations were negatively associated, and FLT3-TKD, IDH1, RUNX1, TET2, TP53 and WT1 were not associated (FIG. 12D). As NPM1, DNMT3A, IDH2 and CEBPA^{bi} mutations are associated with aberrant methylation profiles (Figueroa et al., 2010a; Figueroa et al., 2010b; Ley et al., 2013), their correlation with elevated HE-TSAs^{hi} counts supports the implication of epigenetic dysregulations in TSAs^{hi} expression.

TABLE 6

List of GO terms positively or negatively correlated with HE-TSAshi counts in Leucegene cohort.

| Pos/Neg correlated? | GO term ID | p-value | Hit count in query list | Total genes in GO term | Name |
|---|---|---|---|---|---|
| Negatively | 278 | 5.63E-19 | 74 | 381 | mitotic cell cycle |
| Negatively | 9987 | 2.48E-13 | 599 | 9339 | cellular process |
| Negatively | 51443 | 7.60E-13 | 27 | 71 | positive regulation of ubiquitin-protein ligase activity |
| Negatively | 280 | 2.14E-12 | 48 | 232 | nuclear division |
| Negatively | 7067 | 2.14E-12 | 48 | 232 | mitosis |
| Negatively | 7049 | 2.48E-12 | 100 | 795 | cell cycle |
| Negatively | 51351 | 2.61E-12 | 27 | 74 | positive regulation of ligase activity |
| Negatively | 22402 | 2.72E-12 | 82 | 583 | cell cycle process |
| Negatively | 6091 | 5.20E-12 | 56 | 312 | generation of precursor metabolites and energy |
| Negatively | 87 | 7.37E-12 | 48 | 239 | M phase of mitotic cell cycle |
| Negatively | 51437 | 7.83E-12 | 25 | 65 | positive regulation of ubiquitin-protein ligase activity involved in mitotic cell cycle |
| Negatively | 48285 | 1.04E-11 | 48 | 241 | organelle fission |
| Negatively | 6996 | 2.30E-11 | 142 | 1377 | organelle organization |
| Negatively | 51301 | 2.86E-11 | 55 | 314 | cell division |
| Negatively | 51439 | 4.07E-11 | 25 | 69 | regulation of ubiquitin-protein ligase activity involved in mitotic cell cycle |
| Negatively | 51438 | 5.09E-11 | 27 | 82 | regulation of ubiquitin-protein ligase activity |
| Negatively | 51340 | 1.40E-10 | 27 | 85 | regulation of ligase activity |
| Negatively | 51436 | 4.26E-10 | 23 | 63 | negative regulation of ubiquitin-protein ligase activity involved in mitotic cell cycle |
| Negatively | 31398 | 5.15E-10 | 28 | 96 | positive regulation of protein ubiquitination |
| Negatively | 31145 | 6.32E-10 | 23 | 64 | anaphase-promoting complex-dependent proteasomal ubiquitin-dependent protein catabolic process |
| Negatively | 51352 | 1.97E-09 | 23 | 67 | negative regulation of ligase activity |
| Negatively | 51444 | 1.97E-09 | 23 | 67 | negative regulation of ubiquitin-protein ligase activity |
| Negatively | 43161 | 2.40E-09 | 33 | 140 | proteasomal ubiquitin-dependent protein catabolic process |
| Negatively | 10498 | 2.40E-09 | 33 | 140 | proteasomal protein catabolic process |
| Negatively | 31396 | 9.12E-09 | 30 | 122 | regulation of protein ubiquitination |
| Negatively | 31397 | 1.24E-08 | 24 | 79 | negative regulation of protein ubiquitination |
| Negatively | 16043 | 1.47E-08 | 212 | 2557 | cellular component organization |
| Negatively | 279 | 4.28E-08 | 53 | 351 | M phase |
| Negatively | 31400 | 4.99E-08 | 31 | 138 | negative regulation of protein modification process |
| Negatively | 44237 | 5.13E-08 | 353 | 4974 | cellular metabolic process |
| Negatively | 8152 | 1.63E-07 | 404 | 5938 | metabolic process |
| Negatively | 22403 | 2.44E-07 | 59 | 435 | cell cycle phase |
| Negatively | 8104 | 3.87E-07 | 97 | 921 | protein localization |
| Negatively | 45184 | 5.51E-07 | 85 | 767 | establishment of protein localization |
| Negatively | 15031 | 6.33E-07 | 84 | 756 | protein transport |
| Negatively | 32269 | 8.96E-07 | 36 | 200 | negative regulation of cellular protein metabolic process |
| Negatively | 51248 | 1.42E-06 | 37 | 213 | negative regulation of protein metabolic process |
| Negatively | 33036 | 3.32E-06 | 108 | 1111 | macromolecule localization |
| Negatively | 6119 | 4.67E-06 | 24 | 102 | oxidative phosphorylation |
| Negatively | 31401 | 4.68E-06 | 38 | 232 | positive regulation of protein modification process |
| Negatively | 51179 | 8.10E-06 | 227 | 2978 | localization |
| Negatively | 16192 | 9.74E-06 | 68 | 589 | vesicle-mediated transport |
| Negatively | 9056 | 9.96E-06 | 99 | 1005 | catabolic process |
| Negatively | 22900 | 1.21E-05 | 25 | 115 | electron transport chain |
| Negatively | 32270 | 3.08E-05 | 42 | 291 | positive regulation of cellular protein metabolic process |
| Negatively | 51234 | 4.69E-05 | 201 | 2606 | establishment of localization |
| Negatively | 31399 | 5.75E-05 | 48 | 366 | regulation of protein modification process |
| Negatively | 6511 | 7.23E-05 | 40 | 277 | ubiquitin-dependent protein catabolic process |
| Negatively | 7059 | 8.48E-05 | 20 | 83 | chromosome segregation |
| Negatively | 43632 | 1.21E-04 | 40 | 282 | modification-dependent macromolecule catabolic process |
| Negatively | 19941 | 1.21E-04 | 40 | 282 | modification-dependent protein catabolic process |
| Negatively | 44092 | 1.50E-04 | 48 | 377 | negative regulation of molecular function |
| Negatively | 51247 | 1.69E-04 | 42 | 308 | positive regulation of protein metabolic process |
| Negatively | 9057 | 1.73E-04 | 58 | 502 | macromolecule catabolic process |
| Negatively | 16044 | 2.11E-04 | 48 | 381 | cellular membrane organization |
| Negatively | 51246 | 2.15E-04 | 68 | 635 | regulation of protein metabolic process |
| Negatively | 51603 | 2.24E-04 | 42 | 311 | proteolysis involved in cellular protein catabolic process |
| Negatively | 61024 | 2.29E-04 | 48 | 382 | membrane organization |
| Negatively | 32268 | 2.56E-04 | 62 | 559 | regulation of cellular protein metabolic process |
| Negatively | 44257 | 2.97E-04 | 42 | 314 | cellular protein catabolic process |
| Negatively | 30163 | 3.43E-04 | 44 | 339 | protein catabolic process |
| Negatively | 51641 | 3.79E-04 | 92 | 977 | cellular localization |
| Negatively | 44265 | 4.64E-04 | 52 | 440 | cellular macromolecule catabolic process |
| Negatively | 46907 | 5.91E-04 | 69 | 665 | intracellular transport |
| Negatively | 34613 | 7.00E-04 | 51 | 433 | cellular protein localization |
| Negatively | 51649 | 8.38E-04 | 83 | 865 | establishment of localization in cell |
| Negatively | 70727 | 8.74E-04 | 51 | 436 | cellular macromolecule localization |
| Negatively | 6810 | 9.25E-04 | 193 | 2572 | transport |
| Negatively | 45333 | 1.17E-03 | 20 | 96 | cellular respiration |
| Negatively | 44248 | 1.28E-03 | 76 | 775 | cellular catabolic process |
| Negatively | 42773 | 1.78E-03 | 15 | 57 | ATP synthesis coupled electron transport |
| Negatively | 42775 | 1.78E-03 | 15 | 57 | mitochondrial ATP synthesis coupled electron transport |
| Negatively | 22904 | 1.91E-03 | 16 | 65 | respiratory electron transport chain |
| Negatively | 55114 | 2.07E-03 | 66 | 646 | oxidation reduction |
| Negatively | 43086 | 6.03E-03 | 38 | 301 | negative regulation of catalytic activity |
| Negatively | 15980 | 6.23E-03 | 24 | 145 | energy derivation by oxidation of organic compounds |
| Negatively | 7010 | 1.18E-02 | 49 | 448 | cytoskeleton organization |
| Negatively | 6886 | 1.75E-02 | 42 | 364 | intracellular protein transport |
| Negatively | 226 | 2.27E-02 | 23 | 145 | microtubule cytoskeleton organization |
| Negatively | 7017 | 2.67E-02 | 32 | 247 | microtubule-based process |
| Negatively | 44093 | 2.95E-02 | 62 | 640 | positive regulation of molecular function |
| Negatively | 16052 | 3.12E-02 | 19 | 107 | carbohydrate catabolic process |
| Negatively | 33043 | 4.83E-02 | 31 | 242 | regulation of organelle organization |
| Negatively | 48193 | 5.93E-02 | 21 | 132 | Golgi vesicle transport |
| Negatively | 6508 | 7.37E-02 | 67 | 730 | proteolysis |
| Negatively | 65009 | 9.69E-02 | 89 | 1063 | regulation of molecular function |
| Negatively | 43085 | 1.04E-01 | 54 | 553 | positive regulation of catalytic activity |
| Negatively | 7033 | 1.34E-01 | 11 | 44 | vacuole organization |
| Negatively | 7264 | 1.39E-01 | 34 | 292 | small GTPase mediated signal transduction |
| Negatively | 6007 | 1.64E-01 | 12 | 53 | glucose catabolic process |
| Negatively | 44238 | 1.93E-01 | 334 | 5270 | primary metabolic process |
| Negatively | 16050 | 2.44E-01 | 12 | 55 | vesicle organization |
| Negatively | 7040 | 2.93E-01 | 8 | 25 | lysosome organization |
| Negatively | 70585 | 2.97E-01 | 9 | 32 | protein localization in mitochondrion |
| Negatively | 6626 | 2.97E-01 | 9 | 32 | protein targeting to mitochondrion |
| Negatively | 910 | 3.26E-01 | 11 | 48 | cytokinesis |
| Negatively | 46365 | 3.60E-01 | 13 | 66 | monosaccharide catabolic process |
| Negatively | 6122 | 3.78E-01 | 4 | 5 | mitochondrial electron transport, ubiquinol to cytochrome c |
| Negatively | 51128 | 4.05E-01 | 51 | 538 | regulation of cellular component organization |
| Negatively | 44267 | 5.10E-01 | 153 | 2146 | cellular protein metabolic process |
| Negatively | 6120 | 6.50E-01 | 10 | 43 | mitochondrial electron transport, NADH to ubiquinone |
| Negatively | 8064 | 7.23E-01 | 12 | 61 | regulation of actin polymerization or depolymerization |
| Negatively | 50790 | 7.39E-01 | 75 | 906 | regulation of catalytic activity |
| Negatively | 51656 | 8.00E-01 | 13 | 71 | establishment of organelle localization |
| Negatively | 70 | 8.23E-01 | 9 | 36 | mitotic sister chromatid segregation |
| Negatively | 44275 | 8.29E-01 | 14 | 81 | cellular carbohydrate catabolic process |
| Negatively | 19320 | 1.01E+00 | 12 | 63 | hexose catabolic process |
| Negatively | 30832 | 1.01E+00 | 12 | 63 | regulation of actin filament length |
| Negatively | 819 | 1.04E+00 | 9 | 37 | sister chromatid segregation |
| Negatively | 19538 | 1.13E+00 | 178 | 2604 | protein metabolic process |
| Negatively | 10324 | 1.18E+00 | 26 | 221 | membrane invagination |
| Negatively | 6897 | 1.18E+00 | 26 | 221 | endocytosis |
| Negatively | 32956 | 1.20E+00 | 15 | 94 | regulation of actin cytoskeleton organization |
| Negatively | 43254 | 1.20E+00 | 15 | 94 | regulation of protein complex assembly |
| Negatively | 30833 | 1.23E+00 | 11 | 55 | regulation of actin filament polymerization |
| Negatively | 10564 | 1.25E+00 | 19 | 138 | regulation of cell cycle process |
| Negatively | 7346 | 1.36E+00 | 22 | 174 | regulation of mitotic cell cycle |
| Negatively | 60316 | 1.49E+00 | 3 | 3 | positive regulation of ryanodine-sensitive calcium-release channel activity |
| Negatively | 51640 | 1.53E+00 | 15 | 96 | organelle localization |
| Negatively | 32271 | 1.60E+00 | 12 | 66 | regulation of protein polymerization |
| Negatively | 44283 | 1.92E+00 | 42 | 443 | small molecule biosynthetic process |
| Negatively | 32970 | 1.94E+00 | 15 | 98 | regulation of actin filament-based process |
| Positively | 45449 | 1.70E-14 | 184 | 2621 | regulation of transcription |
| Positively | 19219 | 6.15E-14 | 201 | 3013 | regulation of nucleobase, nucleoside, nucleotide and nucleic acid metabolic process |
| Positively | 51171 | 7.28E-14 | 202 | 3039 | regulation of nitrogen compound metabolic process |
| Positively | 10468 | 6.82E-13 | 194 | 2926 | regulation of gene expression |
| Positively | 10556 | 1.17E-12 | 191 | 2876 | regulation of macromolecule biosynthetic process |
| Positively | 31326 | 4.39E-12 | 196 | 3020 | regulation of cellular biosynthetic process |
| Positively | 9889 | 1.03E-11 | 196 | 3044 | regulation of biosynthetic process |
| Positively | 80090 | 4.31E-11 | 217 | 3553 | regulation of primary metabolic process |
| Positively | 31323 | 1.80E-10 | 223 | 3735 | regulation of cellular metabolic process |
| Positively | 60255 | 1.97E-10 | 207 | 3375 | regulation of macromolecule metabolic process |
| Positively | 19222 | 6.73E-10 | 229 | 3917 | regulation of metabolic process |
| Positively | 51252 | 1.46E-08 | 130 | 1855 | regulation of RNA metabolic process |
| Positively | 6355 | 2.28E-08 | 127 | 1806 | regulation of transcription, DNA-dependent |
| Positively | 50909 | 5.95E-04 | 12 | 44 | sensory perception of taste |

Finally, it was investigated whether TSAs^{hi} expression could be linked to other clinical features allowing to predict their presence in AML patients, such as French-American-British (FAB) types (FIGS. 12E-H). Strikingly, patients expressing high counts of HE-TSAs^{hi} were respectively overrepresented and underrepresented in M1 and M5 AML. Accordingly, patients having AML without maturation and normal karyotypes presented the highest levels of TSAs^{hi}. It was thus hypothesized that this resulted from the overrepresentation of FAB M1 AML in the samples used to discover the TSAs (9 out of 19) and, as most of the TSAs^{hi} were located in intronic regions (37 / 58, including the ERE-derived TSAs^{hi} located in introns), that this could be explained by the existence of different patterns of intron retention between the FAB types. Accordingly, an unsupervised consensus clustering performed on introns specifically retained in AML showed a clear clustering of patients according to their FAB types (FIG. 7G). Altogether, these data show that TSAs^{hi} expression is linked to intron retention patterns specific to AML subtypes.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

Adamopoulou, E., Tenzer, S., Hillen, N., Klug, P., Rota, I.A., Tietz, S., Gebhardt, M., Stevanovic, S., Schild, H., Tolosa, E., et al. (2013). Exploring the MHC-peptide matrix of central tolerance in the human thymus. Nat Commun 4, 2039.

Andreatta, M., and Nielsen, M. (2016). Gapped sequence alignment using artificial neural networks: application to the MHC class I system. Bioinformatics 32, 511-517.

Audemard, E.O., Gendron, P., Feghaly, A., Lavallée, V.P., Hébert, J., Sauvageau, G., and Lemieux, S. (2019). Targeted variant detection using unaligned RNA-Seq reads. Life Sci Alliance 2.

Audoux, J., Philippe, N., Chikhi, R., Salson, M., Gallopin, M., Gabriel, M., Le Coz, J., Drouineau, E., Commes, T., and Gautheret, D. (2017). DE-kupl: exhaustive capture of biological variation in RNA-seq data through k-mer decomposition. Genome Biology 18, 243.

Avigan, D., and Rosenblatt, J. (2018). Vaccine therapy in hematologic malignancies. Blood 131, 2640-2650.

Bamezai, S., Rawat, V.P., and Buske, C. (2012). Concise review: The Piwi-piRNA axis: pivotal beyond transposon silencing. Stem Cells 30, 2603-2611.

Berlin, C., Kowalewski, D.J., Schuster, H., Mirza, N., Walz, S., Handel, M., Schmid-Horch, B., Salih, H.R., Kanz, L., Rammensee, H.G., et al. (2015). Mapping the HLA ligandome landscape of acute myeloid leukemia: a targeted approach toward peptide-based immunotherapy. Leukemia 29, 647-659.

Boegel, S., Lower, M., Bukur, T., Sorn, P., Castle, J.C., and Sahin, U. (2018). HLA and proteasome expression body map. BMC Med Genomics 11, 36.

Bouwmeester, R., Gabriels, R., Hulstaert, N., Martens, L., and Degroeve, S. (2020). DeepLC can predict retention times for peptides that carry as-yet unseen modifications. bioRxiv, 2020.2003.2028.013003.

Bray, N.L., Pimentel, H., Melsted, P., and Pachter, L. (2016). Near-optimal probabilistic RNA-seq quantification. Nature Biotechnology 34, 525-527.

Chapuis, A.G., Egan, D.N., Bar, M., Schmitt, T.M., McAfee, M.S., Paulson, K.G., Voillet, V., Gottardo, R., Ragnarsson, G.B., Bleakley, M., et al. (2019). T cell receptor gene therapy targeting WT1 prevents acute myeloid leukemia relapse post-transplant. Nat Med 25, 1064-1072.

Charni, S., de Bettignies, G., Rathore, M.G., Aguiló, J.I., van den Elsen, P.J., Haouzi, D., Hipskind, R.A., Enriquez, J.A., Sanchez-Beato, M., Pardo, J., et al. (2010). Oxidative Phosphorylation Induces De Novo Expression of the MHC Class I in Tumor Cells through the ERK5 Pathway. The Journal of Immunology 185, 3498-3503.

Chen, J., Brunner, A.D., Cogan, J.Z., Nunez, J.K., Fields, A.P., Adamson, B., Itzhak, D.N., Li, J.Y., Mann, M., Leonetti, M.D., and Weissman, J.S. (2020). Pervasive functional translation of noncanonical human open reading frames. Science 367, 1140-1146.

Chong, C., Muller, M., Pak, H., Harnett, D., Huber, F., Grun, D., Leleu, M., Auger, A., Arnaud, M., Stevenson, B.J., et al. (2020). Integrated proteogenomic deep sequencing and analytics accurately identify non-canonical peptides in tumor immunopeptidomes. Nat Commun 11, 1293.

Corces, M.R., Buenrostro, J.D., Wu, B., Greenside, P.G., Chan, S.M., Koenig, J.L., Snyder, M.P., Pritchard, J.K., Kundaje, A., Greenleaf, W.J., et al. (2016). Lineage-specific and single-cell chromatin accessibility charts human hematopoiesis and leukemia evolution. Nat Genet 48, 1193-1203. Cosma, G.L., and Eisenlohr, L.C. (2019). Impact of epitope density on CD8(+) T cell development and function. Mol Immunol 113, 120-125.

Coulie, P.G., Van den Eynde, B.J., van der Bruggen, P., and Boon, T. (2014). Tumour antigens recognized by T lymphocytes: at the core of cancer immunotherapy. Nat Rev Cancer 14, 135-146. Courcelles, M., Durette, C., Daouda, T., Laverdure, J.P., Vincent, K., Lemieux, S., Perreault, C., and Thibault, P. (2020). MAPDP: A Cloud-Based Computational Platform for Immunopeptidomics Analyses. J Proteome Res.

Danilova, L., Anagnostou, V., Caushi, J.X., Sidhom, J.W., Guo, H., Chan, H.Y., Suri, P., Tam, A., Zhang, J., Asmar, M.E., et al. (2018). The Mutation-Associated Neoantigen Functional Expansion of Specific T Cells (MANAFEST) Assay: A Sensitive Platform for Monitoring Antitumor Immunity. Cancer Immunol Res 6, 888-899.

Daouda, T., Perreault, C., and Lemieux, S. (2016). pyGeno: A Python package for precision medicine and proteogenomics. F1000Res 5, 381.

Di Stasi, A., Jimenez, A.M., Minagawa, K., Al-Obaidi, M., and Rezvani, K. (2015). Review of the Results of WT1 Peptide Vaccination Strategies for Myelodysplastic Syndromes and Acute Myeloid Leukemia from Nine Different Studies. Frontiers in Immunology 6.

Dobin, A., Davis, C.A., Schlesinger, F., Drenkow, J., Zaleski, C., Jha, S., Batut, P., Chaisson, M., and Gingeras, T.R. (2013). STAR: ultrafast universal RNA-seq aligner. Bioinformatics 29, 15-21.

Dutoit, V., Rubio-Godoy, V., Pittet, M.J., Zippelius, A., Dietrich, P.Y., Legal, F.A., Guillaume, P., Romero, P., Cerottini, J.C., Houghten, R.A., et al. (2002). Degeneracy of antigen recognition as the molecular basis for the high frequency of naive A2/Melan-a peptide multimer(+) CD8(+) T cells in humans. J Exp Med 196, 207-216.

Dvinge, H., and Bradley, R.K. (2015). Widespread intron retention diversifies most cancer transcriptomes. Genome medicine 7, 45-45.

Efremova, M., Finotello, F., Rieder, D., and Trajanoski, Z. (2017). Neoantigens Generated by Individual Mutations and Their Role in Cancer Immunity and Immunotherapy. Front Immunol 8, 1679. Egen, J.G., Ouyang, W., and Wu, L.C. (2020). Human Anti-tumor Immunity: Insights from Immunotherapy Clinical Trials. Immunity 52, 36-54.

Ehx, G., and Perreault, C. (2019). Discovery and characterization of actionable tumor antigens. Genome Medicine 11, 29.

Elias, J.E., and Gygi, S.P. (2010). Target-decoy search strategy for mass spectrometry-based proteomics. Methods Mol Biol 604, 55-71.

Eng, J.K., Hoopmann, M.R., Jahan, T.A., Egertson, J.D., Noble, W.S., and MacCoss, M.J. (2015). A deeper look into Comet--implementation and features. J Am Soc Mass Spectrom 26, 1865-1874. Eppert, K., Takenaka, K., Lechman, E.R., Waldron, L., Nilsson, B., van Galen, P., Metzeler, K.H., Poeppl, A., Ling, V., Beyene, J., et al. (2011). Stem cell gene expression programs influence clinical outcome in human leukemia. Nat Med 17, 1086-1093.

Fennell, K.A., Bell, C.C., and Dawson, M.A. (2019). Epigenetic therapies in acute myeloid leukemia: where to from here? Blood 134, 1891-1901.

Fergusson, J.R., Morgan, M.D., Bruchard, M., Huitema, L., Heesters, B.A., van Unen, V., van Hamburg, J.P., van der Wel, N.N., Picavet, D., Koning, F., et al. (2018). Maturing Human CD127+ CCR7+ PDL1+ Dendritic Cells Express AIRE in the Absence of Tissue Restricted Antigens. Front Immunol 9, 2902.

Figueroa, M.E., Abdel-Wahab, O., Lu, C., Ward, P.S., Patel, J., Shih, A., Li, Y., Bhagwat, N., Vasanthakumar, A., Fernandez, H.F., et al. (2010a). Leukemic IDH1 and IDH2 mutations result in a hypermethylation phenotype, disrupt TET2 function, and impair hematopoietic differentiation. Cancer Cell 18, 553-567.

Figueroa, M.E., Lugthart, S., Li, Y., Erpelinck-Verschueren, C., Deng, X., Christos, P.J., Schifano, E., Booth, J., van Putten, W., Skrabanek, L., et al. (2010b). DNA methylation signatures identify biologically distinct subtypes in acute myeloid leukemia. Cancer Cell 17, 13-27.

Garrison, E., and Marth, G. (2012). Haplotype-based variant detection from short-read sequencing. arXiv: Genomics.

Gaujoux, R., and Seoighe, C. (2010). A flexible R package for nonnegative matrix factorization. BMC Bioinformatics 11, 367.

Greiner, J., Hofmann, S., Schmitt, M., Götz, M., Wiesneth, M., Schrezenmeier, H., Bunjes, D., Döhner, H., and Bullinger, L. (2017). Acute myeloid leukemia with mutated nucleophosmin 1: an immunogenic acute myeloid leukemia subtype and potential candidate for immune checkpoint inhibition. Haematologica 102, e499-e501.

Gu, M., Zwiebel, M., Ong, S.H., Boughton, N., Nomdedeu, J., Basheer, F., Nannya, Y., Quiros, P.M., Ogawa, S., Cazzola, M., et al. (2020). RNAmut: robust identification of somatic mutations in acute myeloid leukemia using RNA-sequencing. Haematologica 105, e290-e293.

Gutierrez, S.E., and Romero-Oliva, F.A. (2013). Epigenetic changes: a common theme in acute myelogenous leukemogenesis. J Hematol Oncol 6, 57.

Hardy, M.-P., Vincent, K., and Perreault, C. (2019). The Genomic Landscape of Antigenic Targets for T Cell-Based Leukemia Immunotherapy. Frontiers in Immunology 10, 2934.

He, H., Kondo, Y., Ishiyama, K., Alatrash, G., Lu, S., Cox, K., Qiao, N., Clise-Dwyer, K., St John, L., Sukhumalchandra, P., et al. (2020). Two unique HLA-A*0201 restricted peptides derived from cyclin E as immunotherapeutic targets in leukemia. Leukemia.

Hesnard, L., Legoux, F., Gautreau, L., Moyon, M., Baron, O., Devilder, M.C., Bonneville, M., and Saulquin, X. (2016). Role of the MHC restriction during maturation of antigen-specific human T cells in the thymus. Eur J Immunol 46, 560-569.

Janelle, V., Carli, C., Taillefer, J., Orio, J., and Delisle, J.S. (2015). Defining novel parameters for the optimal priming and expansion of minor histocompatibility antigen-specific T cells in culture. J Transl Med 13, 123.

Jung, N., Dai, B., Gentles, A.J., Majeti, R., and Feinberg, A.P. (2015). An LSC epigenetic signature is largely mutation independent and implicates the HOXA cluster in AML pathogenesis. Nature Communications 6, 8489.

Knaus, H.A., Berglund, S., Hackl, H., Blackford, A.L., Zeidner, J.F., Montiel-Esparza, R., Mukhopadhyay, R., Vanura, K., Blazar, B.R., Karp, J.E., et al. (2018). Signatures of CD8+ T cell dysfunction in AML patients and their reversibility with response to chemotherapy. JCI Insight 3. Krokhin, O.V. (2006). Sequence-specific retention calculator. Algorithm for peptide retention prediction in ion-pair RP-HPLC: application to 300- and 100-A pore size C18 sorbents. Anal Chem 78, 7785-7795.

Lamoliatte, F., McManus, F.P., Maarifi, G., Chelbi-Alix, M.K., and Thibault, P. (2017). Uncovering the SUMOylation and ubiquitylation crosstalk in human cells using sequential peptide immunopurification. Nat Commun 8, 14109.

Larouche, J.D., Trofimov, A., Hesnard, L., Ehx, G., Zhao, Q., Vincent, K., Durette, C., Gendron, P., Laverdure, J.P., Bonneil, E., et al. (2020). Widespread and tissue-specific expression of endogenous retroelements in human somatic tissues. Genome Med 12, 40.

Laumont, C.M., Daouda, T., Laverdure, J.P., Bonneil, E., Caron-Lizotte, O., Hardy, M.P., Granados, D.P., Durette, C., Lemieux, S., Thibault, P., and Perreault, C. (2016). Global proteogenomic analysis of human MHC class I-associated peptides derived from non-canonical reading frames. Nat Commun 7, 10238.

Laumont, C.M., Vincent, K., Hesnard, L., Audemard, E., Bonneil, E., Laverdure, J.P., Gendron, P., Courcelles, M., Hardy, M.P., Cote, C., et al. (2018). Noncoding regions are the main source of targetable tumor-specific antigens. Sci Transl Med 10.

Lavallee, V.P., Baccelli, I., Krosl, J., Wilhelm, B., Barabe, F., Gendron, P., Boucher, G., Lemieux, S., Marinier, A., Meloche, S., et al. (2015). The transcriptomic landscape and directed chemical interrogation of MLL-rearranged acute myeloid leukemias. Nat Genet 47, 1030-1037.

Lavallee, V.P., Krosl, J., Lemieux, S., Boucher, G., Gendron, P., Pabst, C., Boivin, I., Marinier, A., Guidos, C.J., Meloche, S., et al. (2016). Chemo-genomic interrogation of CEBPA mutated AML reveals recurrent CSF3R mutations and subgroup sensitivity to JAK inhibitors. Blood 127, 3054-3061. Lawrence, M.S., Stojanov, P., Polak, P., Kryukov, G.V., Cibulskis, K., Sivachenko, A., Carter, S.L., Stewart, C., Mermel, C.H., Roberts, S.A., et al. (2013). Mutational heterogeneity in cancer and the search for new cancer-associated genes. Nature 499, 214-218.

Legat, A., Maby-EI Hajjami, H., Baumgaertner, P., Cagnon, L., Abed Maillard, S., Geldhof, C., lancu, E.M., Lebon, L., Guillaume, P., Dojcinovic, D., et al. (2016). Vaccination with LAG-31g (IMP321) and Peptides Induces Specific CD4 and CD8 T-Cell Responses in Metastatic Melanoma Patients--Report of a Phase l/lla Clinical Trial. Clin Cancer Res 22, 1330-1340.

Lewinsky, H., Barak, A.F., Huber, V., Kramer, M.P., Radomir, L., Sever, L., Orr, I., Mirkin, V., Dezorella, N., Shapiro, M., et al. (2018). CD84 regulates PD-1/PD-L1 expression and function in chronic lymphocytic leukemia. J Clin Invest 128, 5465-5478.

Ley, T.J., Miller, C., Ding, L., Raphael, B.J., Mungall, A.J., Robertson, A., Hoadley, K., Triche, T.J., Jr., Laird, P.W., Baty, J.D., et al. (2013). Genomic and epigenomic landscapes of adult de novo acute myeloid leukemia. N Engl J Med 368, 2059-2074.

Li, B., Li, T., Wang, B., Dou, R., Zhang, J., Liu, J.S., and Liu, X.S. (2017). Ultrasensitive detection of TCR hypervariable-region sequences in solid-tissue RNA-seq data. Nat Genet 49, 482-483.

Li, H. (2011). A statistical framework for SNP calling, mutation discovery, association mapping and population genetical parameter estimation from sequencing data. Bioinformatics 27, 2987-2993.

Li, H., Handsaker, B., Wysoker, A., Fennell, T., Ruan, J., Homer, N., Marth, G., Abecasis, G., and Durbin, R. (2009). The Sequence Alignment/Map format and SAMtools. Bioinformatics 25, 2078-2079.

Li, S., Garrett-Bakelman, F.E., Chung, S.S., Sanders, M.A., Hricik, T., Rapaport, F., Patel, J., Dillon, R., Vijay, P., Brown, A.L., et al. (2016). Distinct evolution and dynamics of epigenetic and genetic heterogeneity in acute myeloid leukemia. Nat Med 22, 792-799.

Loffler, M.W., Mohr, C., Bichmann, L., Freudenmann, L.K., Walzer, M., Schroeder, C.M., Trautwein, N., Hilke, F.J., Zinser, R.S., Muhlenbruch, L., et al. (2019). Multi-omics discovery of exome-derived neoantigens in hepatocellular carcinoma. Genome Med 11, 28.

Logtenberg, M.E.W., Scheeren, F.A., and Schumacher, T.N. (2020). The CD47-SIRPα Immune Checkpoint. Immunity 52, 742-752.

Luo, K., Yuan, J., Shan, Y., Li, J., Xu, M., Cui, Y., Tang, W., Wan, B., Zhang, N., Wu, Y., and Yu, L. (2006). Activation of transcriptional activities of AP1 and SRE by a novel zinc finger protein ZNF445. Gene 367, 89-100.

Macrae, T., Sargeant, T., Lemieux, S., Hebert, J., Deneault, E., and Sauvageau, G. (2013). RNA-Seq reveals spliceosome and proteasome genes as most consistent transcripts in human cancer cells. PLoS One 8, e72884.

Maere, S., Heymans, K., and Kuiper, M. (2005). BiNGO: a Cytoscape plugin to assess overrepresentation of gene ontology categories in biological networks. Bioinformatics 21, 3448-3449.

Maiga, A., Lemieux, S., Pabst, C., Lavallee, V.P., Bouvier, M., Sauvageau, G., and Hebert, J. (2016). Transcriptome analysis of G protein-coupled receptors in distinct genetic subgroups of acute myeloid leukemia: identification of potential disease-specific targets. Blood Cancer J 6, e431.

Marcais, G., and Kingsford, C. (2011). A fast, lock-free approach for efficient parallel counting of occurrences of k-mers. Bioinformatics 27, 764-770.

Maslak, P.G., Dao, T., Bernal, Y., Chanel, S.M., Zhang, R., Frattini, M., Rosenblat, T., Jurcic, J.G., Brentjens, R.J., Arcila, M.E., et al. (2018). Phase 2 trial of a multivalent WT1 peptide vaccine (galinpepimut-S) in acute myeloid leukemia. Blood Adv 2, 224-234.

Merico, D., Isserlin, R., Stueker, O., Emili, A., and Bader, G.D. (2010). Enrichment map: a network-based method for gene-set enrichment visualization and interpretation. PLoS One 5, e13984.

Middleton, R., Gao, D., Thomas, A., Singh, B., Au, A., Wong, J.J., Bomane, A., Cosson, B., Eyras, E., Rasko, J.E., and Ritchie, W. (2017). IRFinder: assessing the impact of intron retention on mammalian gene expression. Genome Biol 18, 51.

Ntziachristos, P., Abdel-Wahab, O., and Aifantis, I. (2016). Emerging concepts of epigenetic dysregulation in hematological malignancies. Nat Immunol 17, 1016-1024.

Ogishi, M., and Yotsuyanagi, H. (2019). Quantitative Prediction of the Landscape of T Cell Epitope Immunogenicity in Sequence Space. Frontiers in Immunology 10.

Pabst, C., Bergeron, A., Lavallee, V.P., Yeh, J., Gendron, P., Norddahl, G.L., Krosl, J., Boivin, I., Deneault, E., Simard, J., et al. (2016). GPR56 identifies primary human acute myeloid leukemia cells with high repopulating potential in vivo. Blood 127, 2018-2027.

Papaemmanuil, E., Gerstung, M., Bullinger, L., Gaidzik, V.I., Paschka, P., Roberts, N.D., Potter, N.E., Heuser, M., Thol, F., Bolli, N., et al. (2016). Genomic Classification and Prognosis in Acute Myeloid Leukemia. N Engl J Med 374, 2209-2221.

Pearson, H., Daouda, T., Granados, D.P., Durette, C., Bonneil, E., Courcelles, M., Rodenbrock, A., Laverdure, J.P., Cote, C., Mader, S., et al. (2016). MHC class I-associated peptides derive from selective regions of the human genome. J Clin Invest 126, 4690-4701.

Qazilbash, M.H., Wieder, E., Thall, P.F., Wang, X., Rios, R., Lu, S., Kanodia, S., Ruisaard, K.E., Giralt, S.A., Estey, E.H., et al. (2017). PR1 peptide vaccine induces specific immunity with clinical responses in myeloid malignancies. Leukemia 31, 697-704.

Quinlan, A.R., and Hall, I.M. (2010). BEDTools: a flexible suite of utilities for comparing genomic features. Bioinformatics 26, 841-842.

Rashidi, A., and Walter, R.B. (2016). Antigen-specific immunotherapy for acute myeloid leukemia: where are we now, and where do we go from here? Expert Rev Hematol 9, 335-350.

Ritchie, M.E., Phipson, B., Wu, D., Hu, Y., Law, C.W., Shi, W., and Smyth, G.K. (2015). limma powers differential expression analyses for RNA-sequencing and microarray studies. Nucleic Acids Res 43, e47.

Robinson, J.T., Thorvaldsdottir, H., Winckler, W., Guttman, M., Lander, E.S., Getz, G., and Mesirov, J.P. (2011). Integrative genomics viewer. Nat Biotechnol 29, 24-26.

Robinson, M.D., McCarthy, D.J., and Smyth, G.K. (2010). edgeR: a Bioconductor package for differential expression analysis of digital gene expression data. Bioinformatics 26, 139-140.

Rogers, M.F., Shihab, H.A., Mort, M., Cooper, D.N., Gaunt, T.R., and Campbell, C. (2018). FATHMM-XF: accurate prediction of pathogenic point mutations via extended features. Bioinformatics 34, 511-513.

Sarkizova, S., Klaeger, S., Le, P.M., Li, L.W., Oliveira, G., Keshishian, H., Hartigan, C.R., Zhang, W., Braun, D.A., Ligon, K.L., et al. (2020). A large peptidome dataset improves HLA class I epitope prediction across most of the human population. Nat Biotechnol 38, 199-209.

Schulz, W.A., Steinhoff, C., and Florl, A.R. (2006). Methylation of endogenous human retroelements in health and disease. Curr Top Microbiol Immunol 310, 211-250.

Shannon, P., Markiel, A., Ozier, O., Baliga, N.S., Wang, J.T., Ramage, D., Amin, N., Schwikowski, B., and Ideker, T. (2003). Cytoscape: a software environment for integrated models of biomolecular interaction networks. Genome Res 13, 2498-2504.

Shao, W., Pedrioli, P.G.A., Wolski, W., Scurtescu, C., Schmid, E., Vizcaino, J.A., Courcelles, M., Schuster, H., Kowalewski, D., Marino, F., et al. (2018). The SysteMHC Atlas project. Nucleic Acids Res 46, D1237-d1247.

Shlush, L.I., Mitchell, A., Heisler, L., Abelson, S., Ng, S.W.K., Trotman-Grant, A., Medeiros, J.J.F., Rao-Bhatia, A., Jaciw-Zurakowsky, I., Marke, R., et al. (2017). Tracing the origins of relapse in acute myeloid leukaemia to stem cells. Nature 547, 104-108.

Smart, A.C., Margolis, C.A., Pimentel, H., He, M.X., Miao, D., Adeegbe, D., Fugmann, T., Wong, K.-K., and Van Allen, E.M. (2018). Intron retention is a source of neoepitopes in cancer. Nature Biotechnology 36, 1056-1058.

Smith, C.C., Selitsky, S.R., Chai, S., Armistead, P.M., Vincent, B.G., and Serody, J.S. (2019). Alternative tumour-specific antigens. Nat Rev Cancer 19, 465-478.

Springer, I., Besser, H., Tickotsky-Moskovitz, N., Dvorkin, S., and Louzoun, Y. (2020). Prediction of Specific TCR-Peptide Binding From Large Dictionaries of TCR-Peptide Pairs. Front Immunol 11, 1803.

Szolek, A., Schubert, B., Mohr, C., Sturm, M., Feldhahn, M., and Kohlbacher, O. (2014). OptiType: precision HLA typing from next-generation sequencing data. Bioinformatics 30, 3310-3316.

Takahashi, N., Coluccio, A., Thorball, C.W., Planet, E., Shi, H., Offner, S., Turelli, P., Imbeault, M., Ferguson-Smith, A.C., and Trono, D. (2019). ZNF445 is a primary regulator of genomic imprinting. Genes Dev 33, 49-54.

Tamura, H., Dan, K., Tamada, K., Nakamura, K., Shioi, Y., Hyodo, H., Wang, S.D., Dong, H., Chen, L., and Ogata, K. (2005). Expression of functional B7-H2 and B7.2 costimulatory molecules and their prognostic implications in de novo acute myeloid leukemia. Clin Cancer Res 11, 5708-5717.

Tate, J.G., Bamford, S., Jubb, H.C., Sondka, Z., Beare, D.M., Bindal, N., Boutselakis, H., Cole, C.G., Creatore, C., Dawson, E., et al. (2018). COSMIC: the Catalogue Of Somatic Mutations In Cancer. Nucleic Acids Research 47, D941-D947.

Thomsen, M., Lundegaard, C., Buus, S., Lund, O., and Nielsen, M. (2013). MHCcluster, a method for functional clustering of MHC molecules. Immunogenetics 65, 655-665.

Toffalori, C., Zito, L., Gambacorta, V., Riba, M., Oliveira, G., Bucci, G., Barcella, M., Spinelli, O., Greco, R., Crucitti, L., et al. (2019). Immune signature drives leukemia escape and relapse after hematopoietic cell transplantation. Nat Med 25, 603-611.

Tripathi, S.C., Peters, H.L., Taguchi, A., Katayama, H., Wang, H., Momin, A., Jolly, M.K., Celiktas, M., Rodriguez-Canales, J., Liu, H., et al. (2016). Immunoproteasome deficiency is a feature of non-small cell lung cancer with a mesenchymal phenotype and is associated with a poor outcome. Proc Natl Acad Sci U S A 113, E1555-1564.

van der Lee, D.I., Reijmers, R.M., Honders, M.W., Hagedoorn, R.S., de Jong, R.C., Kester, M.G., van der Steen, D.M., de Ru, A.H., Kweekel, C., Bijen, H.M., et al. (2019). Mutated nucleophosmin 1 as immunotherapy target in acute myeloid leukemia. J Clin Invest 129, 774-785.

Vasu, S., Kohlschmidt, J., Mrozek, K., Eisfeld, A.K., Nicolet, D., Sterling, L.J., Becker, H., Metzeler, K.H., Papaioannou, D., Powell, B.L., et al. (2018). Ten-year outcome of patients with acute myeloid leukemia not treated with allogeneic transplantation in first complete remission. Blood Adv 2, 1645-1650.

Vizcaino, J.A., Csordas, A., del-Toro, N., Dianes, J.A., Griss, J., Lavidas, I., Mayer, G., Perez-Riverol, Y., Reisinger, F., Ternent, T., et al. (2016). 2016 update of the PRIDE database and its related tools. Nucleic Acids Res 44, D447-456.

Wang, E., Lu, S.X., Pastore, A., Chen, X., Imig, J., Chun-Wei Lee, S., Hockemeyer, K., Ghebrechristos, Y.E., Yoshimi, A., Inoue, D., et al. (2019a). Targeting an RNA-Binding Protein Network in Acute Myeloid Leukemia. Cancer Cell 35, 369-384.e367.

Wang, Q., Ding, H., He, Y., Li, X., Cheng, Y., Xu, Q., Yang, Y., Liao, G., Meng, X., Huang, C., and Li, J. (2019b). NLRC5 mediates cell proliferation, migration, and invasion by regulating the Wnt/beta-catenin signalling pathway in clear cell renal cell carcinoma. Cancer Lett 444, 9-19.

Whiteway, A., Corbett, T., Anderson, R., Macdonald, I., and Prentice, H.G. (2003). Expression of costimulatory molecules on acute myeloid leukaemia blasts may effect duration of first remission. Br J Haematol 120, 442-451.

Wong, J.J.L., Gao, D., Nguyen, T.V., Kwok, C.-T., van Geldermalsen, M., Middleton, R., Pinello, N., Thoeng, A., Nagarajah, R., Holst, J., et al. (2017). Intron retention is regulated by altered MeCP2-mediated splicing factor recruitment. Nature Communications 8, 15134.

Wu, T.D., and Nacu, S. (2010). Fast and SNP-tolerant detection of complex variants and splicing in short reads. Bioinformatics 26, 873-881.

Yang, L., Rau, R., and Goodell, M.A. (2015). DNMT3A in haematological malignancies. Nat Rev Cancer 15, 152-165.

Zhang, J., Hu, X., Wang, J., Sahu, A.D., Cohen, D., Song, L., Ouyang, Z., Fan, J., Wang, B., Fu, J., et al. (2019). Immune receptor repertoires in pediatric and adult acute myeloid leukemia. Genome Med 11, 73.

Zhao, Q., Laverdure, J.P., Lanoix, J., Durette, C., Cote, C., Bonneil, E., Laumont, C.M., Gendron, P., Vincent, K., Courcelles, M., et al. (2020). Proteogenomics Uncovers a Vast Repertoire of Shared Tumor-Specific Antigens in Ovarian Cancer. Cancer Immunol Res.

Zhi, H., Ning, S., Li, X., Li, Y., Wu, W., and Li, X. (2014). A novel reannotation strategy for dissecting DNA methylation patterns of human long intergenic non-coding RNAs in cancers. Nucleic Acids Res 42, 8258-8270.

Zhou, J., and Chng, W.J. (2017). Aberrant RNA splicing and mutations in spliceosome complex in acute myeloid leukemia. Stem Cell Investig 4, 6.

Zhou, Y., Lu, Y., and Tian, W. (2012). Epigenetic features are significantly associated with alternative splicing. BMC Genomics 13, 123.

Wilson CS, Davidson GS, Martin SB, Andries E, Potter J, Harvey R, et al. Gene expression profiling of adult acute myeloid leukemia identifies novel biologic clusters for risk classification and outcome prediction. Blood 2006; 108(2): 685-696.

Liu L, Xu F, Chang C-K, He Q, Wu L-Y, Zhang Z, et al. MYCN contributes to the malignant characteristics of erythroleukemia through EZH2-mediated epigenetic repression of p21. Cell Death & Disease 2017 2017/10/01; 8(10): e3126-e3126.

Yang X, Lu B, Sun X, Han C, Fu C, Xu K, et al. ANP32A regulates histone H3 acetylation and promotes leukemogenesis. Leukemia 2018 2018/07/01; 32(7): 1587-1597.

de Sá Machado Araújo G, da Silva Francisco Junior R, dos Santos Ferreira C, Mozer Rodrigues PT, Terra Machado D, Louvain de Souza T, et al. Maternal 5mCpG Imprints at the PARD6G-AS1 and GCSAML Differentially Methylated Regions Are Decoupled From Parent-of-Origin Expression Effects in Multiple Human Tissues. Frontiers in Genetics 2018 2018-March-01; 9(36).

Lagus H, Klaas M, Juteau S, Elomaa O, Kere J, Vuola J, et al. Discovery of increased epidermal DNAH10 expression after regeneration of dermis in a randomized with-in person trial — reflections on psoriatic inflammation. Scientific Reports 2019 2019/12/13; 9(1): 19136.

Zhang X, Dong W, Zhou H, Li H, Wang N, Miao X, et al. alpha-2,8-Sialyltransferase Is Involved in the Development of Multidrug Resistance via PI3K/Akt Pathway in Human Chronic Myeloid Leukemia. IUBMB Life 2015 Feb; 67(2): 77-87.

Johnson KD, Kong G, Gao X, Chang Y-I, Hewitt KJ, Sanalkumar R, et al. Cis-regulatory mechanisms governing stem and progenitor cell transitions. Science advances 2015; 1(8): e1500503-e1500503.

What is claimed is:

1-3. (canceled)

4. A tumor antigen peptide (TAP) that binds to an HLA-A *02:01 molecule and comprises the amino acid sequence FLLEFKPVS (SEQ ID NO:7), LLSRGLLFRI (SEQ ID NO: 11), LLDNILQSI (SEQ ID N0:27), FLASFVEKTVL (SEQ ID NO:32), ILASHNLTV (SEQ ID NO:33), IQLTSVHLL (SEQ ID NO:34), LELISFLPVL (SEQ ID NO:35), LLLPESPSI (SEQ ID NO:43), ALASHLIEA (SEQ ID NO:51), ALDDITIQL (SEQ ID NO:52), GLYYKLHNV (SEQ ID NO:61), HLLSETPQL (SEQ ID NO:65), KLLEKAFSI (SEQ ID NO:72), SLWGQPAEA (SEQ ID NO:77), KLQDKEIGL (SEQ ID NO: 108), TLNQGINVYI (SEQ ID NO: 119), ALPVALPSL (SEQ ID NO: 123), ALDPLLLRI (SEQ ID NO: 130), KILDVNLRI (SEQ ID NO: 132), SLLSGLLRA (SEQ ID NO: 146), SLDLLPLSI (SEQ ID NO:150), ILLEEQSLI (SEQ ID NO:167), LTSISIRPV (SEQ ID NO:168), TISECPLLI (SEQ ID NO: 169), ILLSNFSSL (SEQ ID NO:171), RMVAYLQQL (SEQ ID NO: 183), or KLNQAFLVL (SEQ ID NO: 188), or a nucleic encoding said TAP.

5-29. (canceled)

30. The TAP or nucleic acid of claim 4, wherein the TAP is encoded by a sequence located a non-protein coding region of the genome.

31. The TAP or nucleic acid of claim 30, wherein said non-protein coding region of the genome is an untranslated transcribed region (UTR).

32. The TAP or nucleic acid of claim 30, wherein said non-protein coding region of the genome is an intron.

33. The TAP or nucleic acid of claim 30, wherein said non-protein coding region of the genome is an intergenic region.

34. A combination comprising at least two of the TAPs or nucleic acids defined in claim 4.

35. The TAP or nucleic acid of claim 3, which is a nucleic acid.

36. The nucleic acid of claim 35, which is an mRNA.

37. A vehicle comprising the TAP or nucleic acid of claim 4.

38. A composition comprising the TAP or nucleic acid of claim 4, and a pharmaceutically acceptable carrier.

39. A vaccine comprising the TAP or nucleic acid of claim 4, and an adjuvant.

40-43. (canceled)

44. An isolated cell expressing at its surface major histocompatibility complex (MHC) class I molecules comprising the TAP of claim 4 in their peptide binding groove.

45-46. (canceled)

47. A T-cell receptor (TCR) that specifically recognizes MHC class I molecules expressed at the surface of the cell of claim 44.

48. The TCR of claim 47, wherein said TCR comprises a TCRbeta (TCRβ) chain comprising a complementary determining region 3 (CDR3) comprising one of the amino acid sequences set forth in SEQ ID NO: 191-219.

49. An isolated cell expressing at its cell surface the TCR of claim 47.

50. (canceled)

51. A cell population comprising at least 0.5% of the isolated cell as defined in claim 49.

52. A method of treating a myeloid leukemia in a subject comprising administering to the subject an effective amount of:
(i) a TAP FLLEFKPVS (SEQ ID NO_{:}7), LLSRGLLFRI (SEQ ID NO: 11), LLDNILQSI (SEQ ID NO_{:}27), FLASFVEKTVL (SEQ ID NO:32), ILASHNLTV (SEQ ID NO:33), IQLTSVHLL (SEQ ID NO:34), LELISFLPVL (SEQ ID NO:35), LLLPESPSI (SEQ ID NO:43), ALASHLIEA (SEQ ID NO_{:}51), ALDDITIQL (SEQ ID NO:52), ALGNTVPAV (SEQ ID NO:53). ALLPAVPSL (SEQ ID NO:54), GLYYKLHNV (SEQ ID NO:61), HLLSETPQL (SEQ ID NO:65), KLLEKAFSI (SEQ ID NO:72), SLWGQPAEA (SEQ ID NO:77), SVFAGVVGV (SEQ ID NO:82), VLVPYEPPQV (SEQ ID NO:86), VLFGGKVSGA (SEQ ID NO:104), KLQDKEIGL (SEQ ID NO:108), TLNQGINVYI (SEQ ID NO:119), ALPVALPSL (SEQ ID NO:123), ALDPLLLRI (SEQ ID NO:130), KILDVNLRI (SEQ ID NO:132), SLLSGLLRA (SEQ ID NO:146), SLDLLPLSI (SEQ ID NO:150), ILLEEPSLI (SEQ ID NO:167), LTSISIRPV (SEQ ID NO:168), TISECPLLI (SEQ ID NO:169), ILLSNFSSL (SEQ ID NO:171), RMVAYLOQL (SEQ ID NO: 183), or KLNQAFLVL (SEQ ID NO: 188), or a nucleic acid encoding said TAP;
(ii) a combination comprising at least two of the TAPs or nucleic acids defined in (i);
(iii) a vehicle comprising the TAP or nucleic acid defined in (i) or the combination defined in (ii);
(iv) a composition comprising the TAP or nucleic acid defined in (i), the combination defined in (ii), or the vehicle defined in (iii);
(v) a vaccine comprising the TAP or nucleic acid defined in (i), the combination defined in (ii), the vehicle defined in (iii) or the composition defined in (iv);
(vi) a cell expressing at its surface (a) MHC class I molecules comprising the TAP defined in (i) or (b) a TCR that specifically the MHC class I molecules defined in (a); or
(vii) a cell population comprising at least 0.5% of the cells defined in (vi)(b).

53. (canceled)

54. The method of claim 52, wherein said myeloid leukemia is acute myeloid leukemia (AML).

55. The method of claim 52, further comprising administering at least one additional antitumor agent or therapy to the subject.

56. The method of claim 55, wherein said at least one additional antitumor agent or therapy is a chemotherapeutic agent, immunotherapy, an immune checkpoint inhibitor, radiotherapy or surgery.

57-67. (canceled)

68. The TAP or nucleic acid of claim 4, wherein said TAP comprises the amino acid sequence KLQDKEIGL (SEQ ID NO: 108), TLNQGINVYI (SEQ ID NO: 119), ALPVALPSL (SEQ ID NO:123), ALDPLLLRI (SEQ ID NO: 130), KILDVNLRI (SEQ ID NO: 132), SLLSGLLRA (SEQ ID NO: 146) or SLDLLPLSI (SEQ ID NO: 150).

69. The vehicle of claim 37, which is a liposome.


SEQUENCE LISTING (219 sequences)

SEQ ID NO: 1 - protein, 9 aa, Homo sapiens
RQISVQASL

SEQ ID NO: 2 - protein, 9 aa, Homo sapiens
DRELRNLEL

SEQ ID NO: 3 - protein, 9 aa, Homo sapiens
GARQQIHSW

SEQ ID NO: 4 - protein, 8 aa, Homo sapiens
SGKLRVAL

SEQ ID NO: 5 - protein, 11 aa, Homo sapiens
RSASSATQVHK

SEQ ID NO: 6 - protein, 10 aa, Homo sapiens
SASSATQVHK

SEQ ID NO: 7 - protein, 9 aa, Homo sapiens
FLLEFKPVS

SEQ ID NO: 8 - protein, 8 aa, Homo sapiens
GPQVRGSI

SEQ ID NO: 9 - protein, 8 aa, Homo sapiens
IRMKAQAL

SEQ ID NO: 10 - protein, 9 aa, Homo sapiens
KIKVFSKVY

SEQ ID NO: 11 - protein, 10 aa, Homo sapiens
LLSRGLLFRI

SEQ ID NO: 12 - protein, 9 aa, Homo sapiens
LPIASASLL

SEQ ID NO: 13 - protein, 9 aa, Homo sapiens
LYFLGHGSI

SEQ ID NO: 14 - protein, 9 aa, Homo sapiens
NPLQLSLSI

SEQ ID NO: 15 - protein, 8 aa, Homo sapiens
DLMLRESL

SEQ ID NO: 16 - protein, 8 aa, Homo sapiens
VTFKLSLF

SEQ ID NO: 17 - protein, 8 aa, Homo sapiens
IALYKQVL

SEQ ID NO: 18 - protein, 9 aa, Homo sapiens
IVATGSLLK

SEQ ID NO: 19 - protein, 9 aa, Homo sapiens
KIKNKTKNK

SEQ ID NO: 20 - protein, 9 aa, Homo sapiens
KLLSLTIYK

SEQ ID NO: 21 - protein, 8 aa, Homo sapiens
NILKKTVL

SEQ ID NO: 22 - protein, 8 aa, Homo sapiens
NPKLKDIL

SEQ ID NO: 23 - protein, 8 aa, Homo sapiens
NQKKVRIL

SEQ ID NO: 24 - protein, 10 aa, Homo sapiens
PFPLVQVEPV

SEQ ID NO: 25 - protein, 8 aa, Homo sapiens
SPQSGPAL

SEQ ID NO: 26 - protein, 9 aa, Homo sapiens
TSRLPKIQK

SEQ ID NO: 27 - protein, 9 aa, Homo sapiens
LLDNILQSI

SEQ ID NO: 28 - protein, 9 aa, Homo sapiens
RLEVRKVIL

SEQ ID NO: 29 - protein, 9 aa, Homo sapiens
LSWGYFLFK

SEQ ID NO: 30 - protein, 9 aa, Homo sapiens
TILPRILTL

SEQ ID NO: 31 - protein, 8 aa, Homo sapiens
EGKIKRNI

SEQ ID NO: 32 - protein, 11 aa, Homo sapiens
FLASFVEKTVL

SEQ ID NO: 33 - protein, 9 aa, Homo sapiens
ILASHNLTV

SEQ ID NO: 34 - protein, 9 aa, Homo sapiens
IQLTSVHLL

SEQ ID NO: 35 - protein, 10 aa, Homo sapiens
LELISFLPVL

SEQ ID NO: 36 - protein, 9 aa, Homo sapiens
NFCMLHQSI

SEQ ID NO: 37 - protein, 8 aa, Homo sapiens
PARPAGPL

SEQ ID NO: 38 - protein, 8 aa, Homo sapiens
PLPIVPAL

SEQ ID NO: 39 - protein, 9 aa, Homo sapiens
SNLIRTGSH

SEQ ID NO: 40 - protein, 8 aa, Homo sapiens
VPAPAQAI

SEQ ID NO: 41 - protein, 9 aa, Homo sapiens
KGHGGPRSW

SEQ ID NO: 42 - protein, 11 aa, Homo sapiens
ITSSAVTTALK

SEQ ID NO: 43 - protein, 9 aa, Homo sapiens
LLLPESPSI

SEQ ID NO: 44 - protein, 9 aa, Homo sapiens
VILIPLPPK

SEQ ID NO: 45 - protein, 9 aa, Homo sapiens
AVLLPKPPK

SEQ ID NO: 46 - protein, 9 aa, Homo sapiens
TQVSMAESI

SEQ ID NO: 47 - protein, 8 aa, Homo sapiens
LNHLRTSI

SEQ ID NO: 48 - protein, 9 aa, Homo sapiens
NTSHLPLIY

SEQ ID NO: 49 - protein, 8 aa, Homo sapiens
SIQRNLSL

SEQ ID NO: 50 - protein, 9 aa, Homo sapiens
NVSSHVHTV

SEQ ID NO: 51 - protein, 9 aa, Homo sapiens
ALASHLIEA

SEQ ID NO: 52 - protein, 9 aa, Homo sapiens
ALDDITIQL

SEQ ID NO: 53 - protein, 9 aa, Homo sapiens
ALGNTVPAV

SEQ ID NO: 54 - protein, 9 aa, Homo sapiens
ALLPAVPSL

SEQ ID NO: 55 - protein, 9 aa, Homo sapiens
APAPPPVAV

SEQ ID NO: 56 - protein, 8 aa, Homo sapiens
APDKKITL

SEQ ID NO: 57 - protein, 9 aa, Homo sapiens
AQMNLLQKY

SEQ ID NO: 58 - protein, 9 aa, Homo sapiens
DQVIRLAGL

SEQ ID NO: 59 - protein, 9 aa, Homo sapiens
ETTSQVRKY

SEQ ID NO: 60 - protein, 9 aa, Homo sapiens
GGSLIHPQW

SEQ ID NO: 61 - protein, 9 aa, Homo sapiens
GLYYKLHNV

SEQ ID NO: 62 - protein, 9 aa, Homo sapiens
GQKPVILTY

SEQ ID NO: 63 - protein, 9 aa, Homo sapiens
GSLDFQRGW

SEQ ID NO: 64 - protein, 9 aa, Homo sapiens
HHLVETLKF

SEQ ID NO: 65 - protein, 9 aa, Homo sapiens
HLLSETPQL

SEQ ID NO: 66 - protein, 9 aa, Homo sapiens
HQLYRASAL

SEQ ID NO: 67 - protein, 10 aa, Homo sapiens
HTDDIENAKY

SEQ ID NO: 68 - protein, 8 aa, Homo sapiens
IAAPILHV

SEQ ID NO: 69 - protein, 9 aa, Homo sapiens
KAFPFHIIF

SEQ ID NO: 70 - protein, 9 aa, Homo sapiens
KATEYVHSL

SEQ ID NO: 71 - protein, 9 aa, Homo sapiens
KFSNVTMLF

SEQ ID NO: 72 - protein, 9 aa, Homo sapiens
KLLEKAFSI

SEQ ID NO: 73 - protein, 9 aa, Homo sapiens
KPMPTKVVF

SEQ ID NO: 74 - protein, 9 aa, Homo sapiens
NVNRPLTMK

SEQ ID NO: 75 - protein, 11 aa, Homo sapiens
REPYELTVPAL

SEQ ID NO: 76 - protein, 10 aa, Homo sapiens
SEAEAAKNAL

SEQ ID NO: 77 - protein, 9 aa, Homo sapiens
SLWGQPAEA

SEQ ID NO: 78 - protein, 11 aa, Homo sapiens
SPADHRGYASL

SEQ ID NO: 79 - protein, 9 aa, Homo sapiens
SPQSAAAEL

SEQ ID NO: 80 - protein, 8 aa, Homo sapiens
SPVVHQSL

SEQ ID NO: 81 - protein, 8 aa, Homo sapiens
SPYRTPVL

SEQ ID NO: 82 - protein, 9 aa, Homo sapiens
SVFAGVVGV

SEQ ID NO: 83 - protein, 9 aa, Homo sapiens
SYSPAHARL

SEQ ID NO: 84 - protein, 9 aa, Homo sapiens
THGSEQLHL

SEQ ID NO: 85 - protein, 9 aa, Homo sapiens
TQAPPNVVL

SEQ ID NO: 86 - protein, 10 aa, Homo sapiens
VLVPYEPPQV

SEQ ID NO: 87 - protein, 9 aa, Homo sapiens
VSFPDVRKV

SEQ ID NO: 88 - protein, 11 aa, Homo sapiens
VVFDKSDLAKY

SEQ ID NO: 89 - protein, 9 aa, Homo sapiens
YSHHSGLEY

SEQ ID NO: 90 - protein, 9 aa, Homo sapiens
YYLDWIHHY

SEQ ID NO: 91 - protein, 9 aa, Homo sapiens
SVYKYLKAK

SEQ ID NO: 92 - protein, 9 aa, Homo sapiens
IYQFIMDRF

SEQ ID NO: 93 - protein, 9 aa, Homo sapiens
GTLQGIRAW

SEQ ID NO: 94 - protein, 10 aa, Homo sapiens
AQKVSVGQAA

SEQ ID NO: 95 - protein, 9 aa, Homo sapiens
LYPSKLTHF

SEQ ID NO: 96 - protein, 9 aa, Homo sapiens
ATQNTIIGK

SEQ ID NO: 97 - protein, 9 aa, Homo sapiens
AQDIILQAV

SEQ ID NO: 98 - protein, 9 aa, Homo sapiens
PPRPLGAQV

SEQ ID NO: 99 - protein, 9 aa, Homo sapiens
FNVALNARY

SEQ ID NO: 100 - protein, 9 aa, Homo sapiens
GPGSRESTL

SEQ ID NO: 101 - protein, 8 aa, Homo sapiens
IPHQRSSL

SEQ ID NO: 102 - protein, 9 aa, Homo sapiens
LTDRIYLTL

SEQ ID NO: 103 - protein, 9 aa, Homo sapiens
NLKEKKALF

SEQ ID NO: 104 - protein, 10 aa, Homo sapiens
VLFGGKVSGA

SEQ ID NO: 105 - protein, 9 aa, Homo sapiens
VVFPFPVNK

SEQ ID NO: 106 - protein, 9 aa, Homo sapiens
SLLIIPKKK

SEQ ID NO: 107 - protein, 9 aa, Homo sapiens
APGAAGQRL

SEQ ID NO: 108 - protein, 9 aa, Homo sapiens
KLQDKEIGL

SEQ ID NO: 109 - protein, 9 aa, Homo sapiens
SLREPQPAL

SEQ ID NO: 110 - protein, 9 aa, Homo sapiens
TPGRSTQAI

SEQ ID NO: 111 - protein, 8 aa, Homo sapiens
APRGTAAL

SEQ ID NO: 112 - protein, 8 aa, Homo sapiens
IASPIALL

SEQ ID NO: 113 - protein, 9 aa, Homo sapiens
ILFQNSALK

SEQ ID NO: 114 - protein, 8 aa, Homo sapiens
ILKKNISI

SEQ ID NO: 115 - protein, 8 aa, Homo sapiens
IPLAVRTI

SEQ ID NO: 116 - protein, 8 aa, Homo sapiens
LPRNKPLL

SEQ ID NO: 117 - protein, 9 aa, Homo sapiens
PAPPHPAAL

SEQ ID NO: 118 - protein, 8 aa, Homo sapiens
SPVVRVGL

SEQ ID NO: 119 - protein, 10 aa, Homo sapiens
TLNQGINVYI

SEQ ID NO: 120 - protein, 9 aa, Homo sapiens
RPRGPRTAP

SEQ ID NO: 121 - protein, 11 aa, Homo sapiens
SVQLLEQAIHK

SEQ ID NO: 122 - protein, 9 aa, Homo sapiens
RTPKNYQHW

SEQ ID NO: 123 - protein, 9 aa, Homo sapiens
ALPVALPSL

SEQ ID NO: 124 - protein, 9 aa, Homo sapiens
SLQILVSSL

SEQ ID NO: 125 - protein, 9 aa, Homo sapiens
ISNKVPKLF

SEQ ID NO: 126 - protein, 10 aa, Homo sapiens
TVIRIAIVNK

SEQ ID NO: 127 - protein, 9 aa, Homo sapiens
KEIFLELRL

SEQ ID NO: 128 - protein, 9 aa, Homo sapiens
TLRSPGSSL

SEQ ID NO: 129 - protein, 9 aa, Homo sapiens
TVRGDVSSL

SEQ ID NO: 130 - protein, 9 aa, Homo sapiens
ALDPLLLRI

SEQ ID NO: 131 - protein, 9 aa, Homo sapiens
ISLIVTGLK

SEQ ID NO: 132 - protein, 9 aa, Homo sapiens
KILDVNLRI

SEQ ID NO: 133 - protein, 9 aa, Homo sapiens
ERVYIRASL

SEQ ID NO: 134 - protein, 8 aa, Homo sapiens
ILDLESRY

SEQ ID NO: 135 - protein, 9 aa, Homo sapiens
KTFVQQKTL

SEQ ID NO: 136 - protein, 9 aa, Homo sapiens
LYIKSLPAL

SEQ ID NO: 137 - protein, 9 aa, Homo sapiens
VLKEKNASL

SEQ ID NO: 138 - protein, 9 aa, Homo sapiens
LGISLTLKY

SEQ ID NO: 139 - protein, 8 aa, Homo sapiens
DLLPKKLL

SEQ ID NO: 140 - protein, 9 aa, Homo sapiens
HSLISIVYL

SEQ ID NO: 141 - protein, 9 aa, Homo sapiens
IAGALRSVL

SEQ ID NO: 142 - protein, 9 aa, Homo sapiens
IGNPILRVL

SEQ ID NO: 143 - protein, 9 aa, Homo sapiens
IYAPHIRLS

SEQ ID NO: 144 - protein, 8 aa, Homo sapiens
LRSQILSY

SEQ ID NO: 145 - protein, 9 aa, Homo sapiens
RYLANKIHI

SEQ ID NO: 146 - protein, 9 aa, Homo sapiens
SLLSGLLRA

SEQ ID NO: 147 - protein, 8 aa, Homo sapiens
SRIHLVVL

SEQ ID NO: 148 - protein, 10 aa, Homo sapiens
SSSPVRGPSV

SEQ ID NO: 149 - protein, 9 aa, Homo sapiens
STFSLYLKK

SEQ ID NO: 150 - protein, 9 aa, Homo sapiens
SLDLLPLSI

SEQ ID NO: 151 - protein, 9 aa, Homo sapiens
VTDLLALTV

SEQ ID NO: 152 - protein, 11 aa, Homo sapiens
RTQITKVSLKK

SEQ ID NO: 153 - protein, 8 aa, Homo sapiens
ILRSPLKW

SEQ ID NO: 154 - protein, 9 aa, Homo sapiens
LSTGHLSTV

SEQ ID NO: 155 - protein, 9 aa, Homo sapiens
TVEEYLVNI

SEQ ID NO: 156 - protein, 10 aa, Homo sapiens
QIKTKLLGSL

SEQ ID NO: 157 - protein, 10 aa, Homo sapiens
LPSFSHFLLL

SEQ ID NO: 158 - protein, 9 aa, Homo sapiens
CLRIGPVTL

SEQ ID NO: 159 - protein, 9 aa, Homo sapiens
HVSDGSTAL

SEQ ID NO: 160 - protein, 9 aa, Homo sapiens
IAYSVRALR

SEQ ID NO: 161 - protein, 8 aa, Homo sapiens
PRGFLSAL

SEQ ID NO: 162 - protein, 9 aa, Homo sapiens
ISSWLISSL

SEQ ID NO: 163 - protein, 9 aa, Homo sapiens
IPLNPFSSL

SEQ ID NO: 164 - protein, 8 aa, Homo sapiens
LSDRQLSL

SEQ ID NO: 165 - protein, 9 aa, Homo sapiens
LSHPAPSSL

SEQ ID NO: 166 - protein, 9 aa, Homo sapiens
LRKAVDPIL

SEQ ID NO: 167 - protein, 9 aa, Homo sapiens
ILLEEQSLI

SEQ ID NO: 168 - protein, 9 aa, Homo sapiens
LTSISIRPV

SEQ ID NO: 169 - protein, 9 aa, Homo sapiens
TISECPLLI

SEQ ID NO: 170 - protein, 9 aa, Homo sapiens
TLKLKKIFF

SEQ ID NO: 171 - protein, 9 aa, Homo sapiens
ILLSNFSSL

SEQ ID NO: 172 - protein, 9 aa, Homo sapiens
LGGAWKAVF

SEQ ID NO: 173 - protein, 9 aa, Homo sapiens
LSASHLSSL

SEQ ID NO: 174 - protein, 9 aa, Homo sapiens
AGDIIARLI

SEQ ID NO: 175 - protein, 9 aa, Homo sapiens
DRGILRNLL

SEQ ID NO: 176 - protein, 9 aa, Homo sapiens
GLRLIHVSL

SEQ ID NO: 177 - protein, 9 aa, Homo sapiens
GLRLLHVSL

SEQ ID NO: 178 - protein, 9 aa, Homo sapiens
LHNEKGLSL

SEQ ID NO: 179 - protein, 11 aa, Homo sapiens
LPSFSRPSGII

SEQ ID NO: 180 - protein, 9 aa, Homo sapiens
LSSRLPLGK

SEQ ID NO: 181 - protein, 8 aa, Homo sapiens
MIGIKRLL

SEQ ID NO: 182 - protein, 8 aa, Homo sapiens
NLKKREIL

SEQ ID NO: 183 - protein, 9 aa, Homo sapiens
RMVAYLQQL

SEQ ID NO: 184 - protein, 9 aa, Homo sapiens
SPARALPSL

SEQ ID NO: 185 - protein, 8 aa, Homo sapiens
TVPGIQRY

SEQ ID NO: 186 - protein, 9 aa, Homo sapiens
VSRNYVLLI

SEQ ID NO: 187 - protein, 9 aa, Homo sapiens
LTVPLSVFW

SEQ ID NO: 188 - protein, 9 aa, Homo sapiens
KLNQAFLVL

SEQ ID NO: 189 - protein, 10 aa, Homo sapiens
RLVSSTLLQK

SEQ ID NO: 190 - protein, 8 aa, Homo sapiens
LPSHSLLI

SEQ ID NO: 191 - protein, 13 aa, Homo sapiens
CSARGDREYEQYF

SEQ ID NO: 192 - protein, 15 aa, Homo sapiens
CASTVVAGNSSPLHF

SEQ ID NO: 193 - protein, 16 aa, Homo sapiens
CASSQDGIWGAYEQYF

SEQ ID NO: 194 - protein, 15 aa, Homo sapiens
CASSVDAGGNYEQYF

SEQ ID NO: 195 - protein, 16 aa, Homo sapiens
CASSLGGQGLSYGYTF

SEQ ID NO: 196 - protein, 12 aa, Homo sapiens
CASSYRPNEQYF

SEQ ID NO: 197 - protein, 14 aa, Homo sapiens
CASSGTDLNQPQHF

SEQ ID NO: 198 - protein, 12 aa, Homo sapiens
CASSSNFEPLHF

SEQ ID NO: 199 - protein, 15 aa, Homo sapiens
CASSWGGSNTGELFF

SEQ ID NO: 200 - protein, 15 aa, Homo sapiens
CASSEYRALNTEAFF

SEQ ID NO: 201 - protein, 15 aa, Homo sapiens
CASSESGTGGQPQHF

SEQ ID NO: 202 - protein, 14 aa, Homo sapiens
CASSRTGENTEAFF

SEQ ID NO: 203 - protein, 15 aa, Homo sapiens
CASSSTDRQHYGYTF

SEQ ID NO: 204 - protein, 17 aa, Homo sapiens
CASSDRTGGSSNEKLFF

SEQ ID NO: 205 - protein, 15 aa, Homo sapiens
CATSRSGDSNQPQHF

SEQ ID NO: 206 - protein, 13 aa, Homo sapiens
CASSYVLNTEAFF

SEQ ID NO: 207 - protein, 15 aa, Homo sapiens
CASRESGQMNEKLFF

SEQ ID NO: 208 - protein, 15 aa, Homo sapiens
CASSDGGEGTYGYTF

SEQ ID NO: 209 - protein, 17 aa, Homo sapiens
CASTSWTGFGPNYGYTF

SEQ ID NO: 210 - protein, 14 aa, Homo sapiens
CASSADSSLGGYTF

SEQ ID NO: 211 - protein, 15 aa, Homo sapiens
CSARDLAGGTYEQYF

SEQ ID NO: 212 - protein, 15 aa, Homo sapiens
CASSPPTGEYEKLFF

SEQ ID NO: 213 - protein, 9 aa, Homo sapiens
CARSFGGFF

SEQ ID NO: 214 - protein, 15 aa, Homo sapiens
CASSLPSGILYEQYF

SEQ ID NO: 215 - protein, 14 aa, Homo sapiens
CASAPGGMPYGYTF

SEQ ID NO: 216 - protein, 15 aa, Homo sapiens
CASSLQDTDYNEQFF

SEQ ID NO: 217 - protein, 18 aa, Homo sapiens
CASSDPGTSGVFTGELFF

SEQ ID NO: 218 - protein, 12 aa, Homo sapiens
CSARLGTGELFF

SEQ ID NO: 219 - protein, 12 aa, Homo sapiens
CSARLGTGELFF